(19) United States
(12) Patent Application Publication
(10) Pub. No.: US 2005/0266410 A1
(43) Pub. Date: Dec. 1, 2005

(54) Methods of Human Leukocyte Antigen typing by neighboring single nucleotide polymorphism haplotypes

(76) Inventors: Emily Walsh, Cambridge, MA (US); John D. Rioux, Cambridge, MA (US); Eric S. Lander, Cambridge, MA (US)

Correspondence Address: FISH & NEAVE IP GROUP;ROPES & GRAY LLP, ONE INTERNATIONAL PLACE, BOSTON, MA, 02110-2624, US

(21) Appl. No.: 10/850,359

(22) Filed: May 19, 2004

(51) Int. Cl. C12Q 1/68; G06F 19/00; G01N 33/48; G01N 33/50

(52) U.S. Cl. 435/6; 702/20

(57) ABSTRACT

The disclosure relates to novel approaches to mapping the MHC region and provides novel methods of genotyping the HLA loci. A haplotype map of the region and methods of using the map are also disclosed.

### STATEMENT REGARDING FEDERAL FUNDING

Work described herein was funded, in whole or in part, from the National Cancer Institute, National Institutes of Health, under contract N01-CO-12400. The United States government has certain rights in the invention.

### BACKGROUND OF THE INVENTION

The classical Human Leukocyte Antigen (HLA) loci are the most highly variable genes in the human genome. Historically, attempts to characterize the region have focused on a handful of highly variable, classical HLA genes (class-I genes: HLA-A, HLA-B, and HLA-C; and class-II genes: HLA-DRB1, HLA-DQA1, HLA-DQB1, HLA-DPA1, and HLA-DPB1). These genes encode antigen-presenting molecules that mediate acquired immune response during infection, as well as host-graft responses after organ transplantation. All organ transplant donors and recipients are typed for these genes in order to best match donor with recipient. Also, these genes have been associated with many human autoimmune and inflammatory diseases, and many research laboratories genotype their human subjects for these loci as a matter of course. The HLA loci were originally studied by lower resolution serotyping techniques until the recent advent of “dot blot” hybridization-based molecular typing such as SSOP and SSP (Dynal Biotech, Biotest, One Lambda) that greatly improved examination of the region. Direct sequencing of HLA alleles is also possible. However, these current methods are laborious and expensive. Accordingly, novel approaches to map the HLA loci in the context of the MHC region are desirable.

### SUMMARY OF THE INVENTION

Accordingly, the invention provides a more uniform, comprehensive map of commonly linked variation, e.g., a haplotype map, that will help to discriminate between causal alleles and variation that is merely in linkage disequilibrium (LD) with them. Such a resource will also allow a more complete description of the haplotype structure and, potentially, insight into the evolutionary and recombinational history of the chromosomal region in question.

The invention provides an integrated SNP-haplotype map of a 4-Mb major histocompatibility complex (MHC) region. Preferably, the integrated map comprises SNPs that are preferred to be reliable, polymorphic, and evenly spaced, e.g., one SNP every 20 kb. The integrated map further comprises genotyped HLA genes, TAP genes, microsatellites, or combination thereof.

The invention further features a novel method of genotyping Human Leukocyte Antigen (HLA) genes using patterns of neighboring single nucleotide polymorphisms (SNPs). The SNP-based method is an improvement over existing hybridization-based techniques, as it allows quick and inexpensive genotyping of the HLA loci. This method does not directly assess the intra-gene variation, as is done by all other current methods for HLA genotyping, but rather define HLA genotypes by studying the neighboring extra-genic variation(s) which falls outside the HLA allele to be genotyped and which, due to LD patterns, is conveniently linked to the HLA loci. Identification of the correlation of this extra-genic variation to the HLA gene alleles allows for the discovery and utilization of surrogate markers for HLA genotypes.

This approach to genotype the HLA loci overcomes a substantial technical difficulty to applying high-throughput genotyping techniques to these hypervariable genes. By focusing on variation outside of the hypervariable HLA genes themselves, this method avoids the pitfalls of polymerase chain reaction (PCR) primer design in locations where nucleotide diversity can be as high as 12% (i.e., an average of 12 base pairs substituted per 100 nucleotides assessed). Instead, ancestral “hitchhiking mutations” outside of these genes are used to resolve HLA genotypes with traditional SNP genotyping methods. This approach can be employed to map variation(s) in the regions neighboring HLA genes to fully resolve all known common HLA gene variants in multiple different ethnic populations. This method can benefit clinical laboratories typing individuals for transplantation procedures, as well as research laboratories that are interested in studying HLA gene variation(s) in particular patient populations or disease associations. Further, this method can be employed to predict the likelihood or probability of developing a disease, particularly MHC-linked diseases or autoimmune diseases. Alternatively, this method can be employed to predict the likelihood or probability of developing an immune response, e.g., a response against infection or a host-graft response (e.g., elicited by organ transplantation) in a subject, preferably a human subject.

One aspect of the invention provides a method of genotyping an HLA gene, such as for example an HLA-A or an HLA-DRB1 gene. The method comprises determining the nucleotide present at one or more extra-genic SNP sites, wherein the SNP is associated with an HLA genotype. The extra-genic SNP sites correspond to the HLA allele to be genotyped, that is, the SNP sites are outside and in the neighboring region(s) of the HLA allele to be genotyped. For example, to genotype the HLA-A allele, an extra-genic SNP to be assessed that corresponds to the HLA-A allele can be rs2517862, rs1655930, rs1616549, rs376253, rs1961135, rs2517706, rs2517701, rs2517699, rs435766, rs410909, rs2394255, rs1264807, rs2530388, rs356963, rs2286405, rs2240619, rs3129012, rs259938, or any combination thereof. Another example involves genotyping the HLA-DRB1 allele, wherein an extra-genic SNP to be assessed can be rs742697, rs523627, rs3129960, rs2395163, rs2395165, rs983561, rs2239804, rs2213584, rs2395182, rs2858860, rs3129907, rs1059544, rs1987529, or any combination thereof.

Another aspect of the invention provides a method of predicting or assisting in predicting the likelihood of developing a disease, in particular an inflammatory disease, an MHC-linked disease, or an autoimmune disease, in a subject, preferably a human subject. The method comprises genotyping an HLA gene in the subject to be tested by determining the nucleotide present at one or more extra-genic SNP sites, wherein the SNP is associated with the HLA genotype.

A further aspect of the invention provides a method of predicting or assisting in predicting the likelihood of developing an immune response in a subject, preferably a human subject. An immune response may be developed against an infection or inflammation. Alternatively, an immune response may comprise a host-graft response, e.g., rejection of organ transplants. The method comprises genotyping an HLA gene in the subject to be tested by determining the nucleotide present at one or more extra-genic SNP sites, wherein the SNP is associated with an HLA genotype.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-1E shows an integrated SNP map of the 4-Mb MHC in CEPH Europeans. FIG. 1A shows the location and exon-intron structure for a subset of genes above the map, for positional reference. FIG. 1B shows 201 reliable, polymorphic SNPs, indicated on the map with ticks below the line. Ticks above the line are placed with 100-kb spacing. FIG. 1C shows haplotype blocks below and common haplotype variants (13% frequency) shown as colored lines (thickness indicates relative population frequency). Colors serve only to distinguish haplotypes and do not indicate block to-block connections. Asterisks are found below the seven largest haplotype blocks. FIG. 1D shows pairwise D′ values (Lewontin 1964) for SNPs indicated below the haplotype blocks. Note that each block represents a single D′ calculation and is placed in the middle between the two SNPs analyzed. Red indicates strong LD and high confidence of the D′ estimate (D′>0.95; LOD>=3.0). Blue indicates strong LD with low confidence of the estimate of D′ (D′=1; LOD<3.0). White indicates weak LD. FIG. 1E, shows the relative recombination rate, which is based on the sperm meiotic map, indicated in bar-graph form, where the value on the line is the regional average, 0.49 cM/Mb. Green bars indicate recombination rates >0.49 cM/Mb, and yellow bars indicate rates <0.49 cM/Mb. The black arrowhead denotes a region of well-mapped recombination rate from Jeffreys et al. (2001). SNP marker density in that region is too low to comment on any similarities between the studies described herein. Note that five of seven long haplotype blocks map to regions where the recombination rate is =<0.49 cM/Mb. The remaining two long blocks are found in domains where recombination rates are 0.64 cM/Mb and 0.83 cM/Mb (rates near or below the genomewide average).

FIG. 2A-2D show block comparison between the MHC and other autosomal regions. FIG. 2A shows a plot of LD by physical distance revealing that LD is extended in the MHC. FIG. 2B shows that the average physical length of blocks in the MHC is longer than in the rest of the genome. FIG. 2C shows that, measured by genetic distance, block size in the MHC is somewhat less than in the rest of the genome. FIG. 2D shows that the number of haplotype variants in blocks not spanning classical HLA genes is the same as elsewhere in the genome.

FIG. 3A-3C shows EHH analysis of haplotype blocks, microsatellites, HLA genes, and TAP genes in the region. EHH is computed as the percentage of instances in which two randomly selected chromosomes with the same variant locus have identical alleles at all SNPs assayed up to a particular distance from that locus (e.g., an EHH of 0.5 at marker X means that 50% of possible pairings of a particular variant exhibit sequence identity from the locus to marker X). FIG. 3A shows points representing the EHH at a distance of 0.25 cM from an allele at a particular locus. Outlying variants are indicated in color. The nine outlying variants define three extended haplotypes. The six points labeled as “1” indicate variants that map on the DRB1*1501 haplotype (associated with lupus and MS). The two overlapping points labeled as “2” indicate variants C*0701 and D6S2840*219, which are both found on a haplotype associated with autoimmune diabetes, lupus, and hepatitis. The point labeled as “3” indicates DRB1*1101 (associated with pemphigoid disease). FIG. 3B shows a recombination-distance-based map of the region. Microsatellites/genes are labeled and indicated with ticks above the line. FIG. 3C shows EHH values for loci that have at least one outlying variant. Outlying variants were seen at 7 of the 48 independent loci tested. The X-axis denotes distance in cM. EHH values are converted to grayscale values: EHH of 1 p black, EHH of 0.5 p 50% grayscale. The solid lines 4-10 indicate the locus about which values were derived. The dotted lines 11-17 and 11′-17′ indicate 0.25-cM distance at which outliers were assessed. Two HLA-C alleles, C*0702 and C*0701, are extended, as are two DRB1 alleles, DRB1*1501 and DRB1*1101. The other HLA gene alleles with extended haplotypes are DQA1*0102 and DQB1*0602. The microsatellite alleles with extended haplotypes are D6S2793*244, D6S2876*11, and D6S2840*219.

FIG. 4A-4B show correlation of HLA alleles to SNP haplotype background. A map of region showing placement of SNPs and haplotypes assayed is shown for reference. Multi-SNP haplotypes are coded by single capital letters. FIG. 4A shows SNP-HLA haplotypes sorted by HLA allele. Percents indicate the percentage of a particular HLA allele that falls on the indicated SNP haplotype. FIG. 4B shows SNP-HLA haplotypes sorted by SNP haplotype allele. Percents indicate the percentage of a particular SNP haplotype allele that bears the indicated HLA allele. Counts are overall number of chromosomes bearing the SNP-HLA haplotype indicated.

### DETAILED DESCRIPTION OF THE INVENTION

Overview

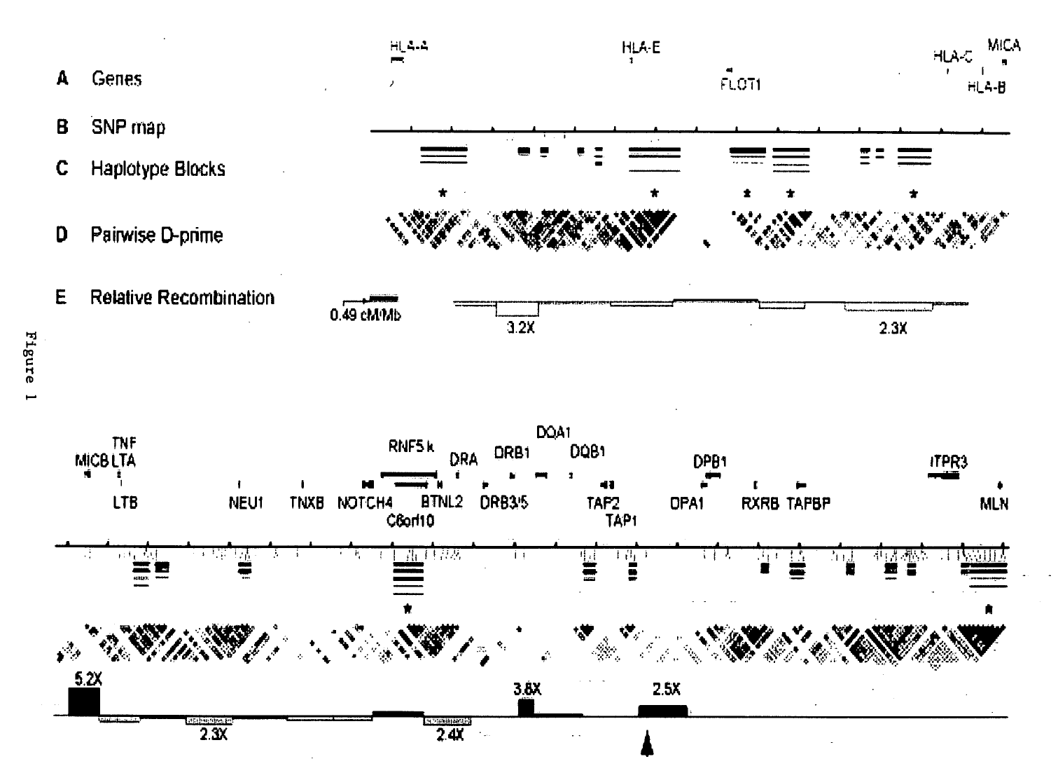

In order to fully map the variation, especially variations associated with diseases, in the MHC region, an SNP haplotype map of the region was created. To be able to integrate this map with the wealth of findings from association studies, 201 reliable, polymorphic, evenly spaced SNPs (target density: one SNP every 20 kb) were genotyped in 136 independent chromosomes also genotyped for nine HLA genes, two TAP genes (involved in antigen processing), and 18 microsatellites. Markers were genotyped in families (18 multigenerational European pedigrees) to allow direct assessment of chromosomal phase and, thus, simple reconstruction of haplotypes. Using these SNP data, the haplotype patterns of the region and mapped these patterns were examined, relative to both genetic and physical distance, as assayed by an exceedingly high-resolution recombination map (FIGS. 1A-1E). This recombination map is the result of the analysis of 20,000 sperm meioses from 12 men (Cullen et al. 2002).

This SNP density is a large first step toward a comprehensive characterization of the patterns of common variation in the MHC. Here, this map is used to first explore the structure of LD in the region, with respect to both haplotype blocks and extended haplotypes. Next, SNP-haplotype variation in the MHC was examined, first considering regions between the classical HLA loci and then examining SNP-haplotype variation across these genes. The question of whether the SNP haplotype diversity near classical HLA loci contained enough information to predict the HLA allele carried on the chromosome was also examined.

FIGS. 1A-1E show an integrated map of the SNP, microsatellite, and HLA variation in the MHC. This map shows that, aside from the classical HLA loci, the variation and LD structure of the MHC are not different from a genomewide control data set. Specifically, whereas LD appears to extend over longer physical distances in the MHC, this seems to be accounted for by the reduced recombination rate in the region. Furthermore, this map shows that, in the regions that do not span classical HLA loci, the number of common haplotype alleles in the MHC are not different from the rest of the genome.

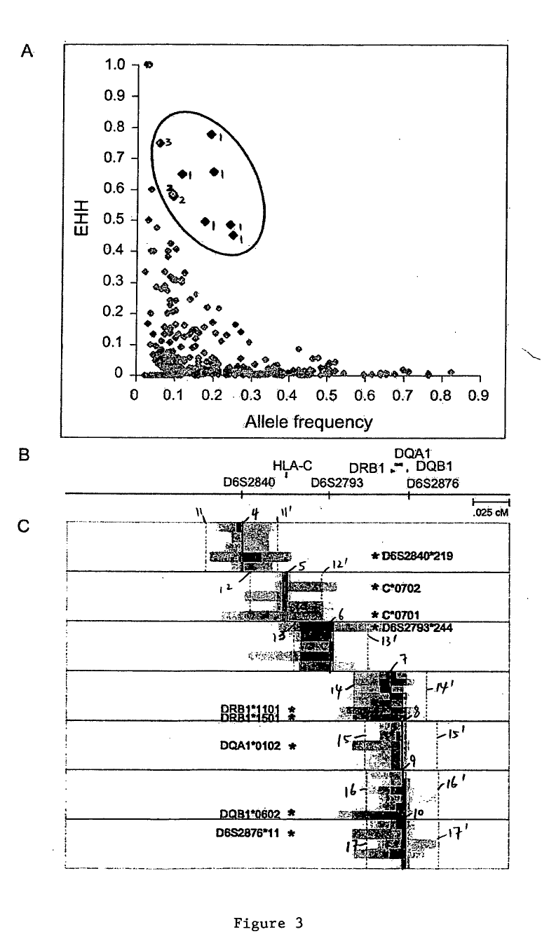

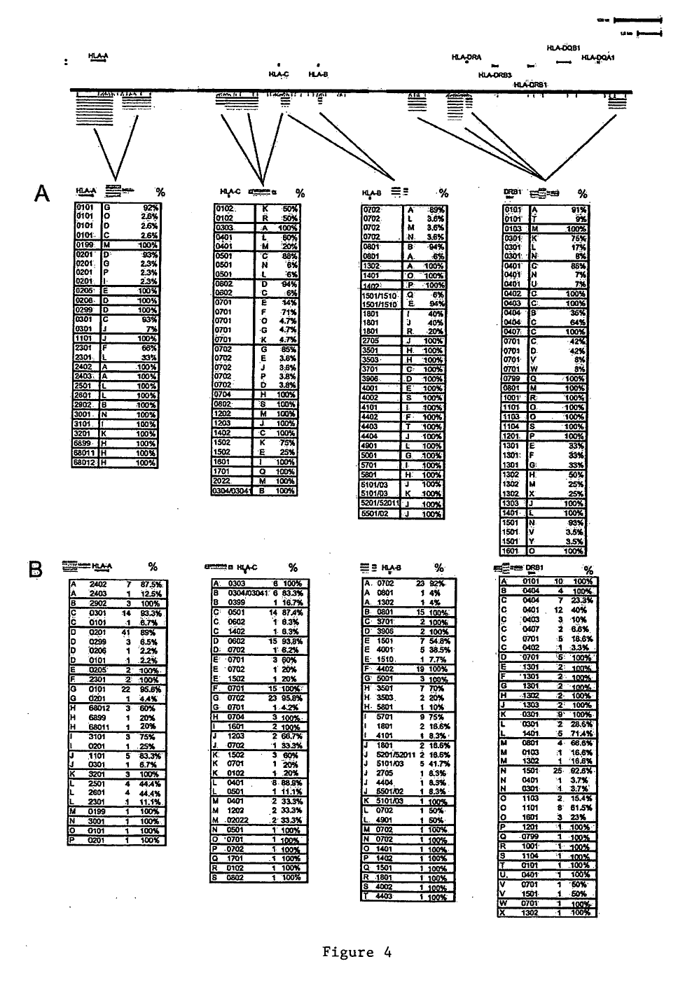

The integrated map of FIGS. 1A-1E and the results shown in FIGS. 3A-3C and 4A-4B demonstrate that multiblock SNP haplotypes contain considerable predictive information for common HLA alleles at HLA-A, HLA-B, HLA-C, and HLA-DRB1. Multiblock SNP haplotypes should enable cost-efficient, large-scale exploration of the variation at the classical HLA loci and beyond. An additional implication of these results is that multiblock SNP haplotypes may be sufficient to identify low-frequency variants throughout the genome. Such low-frequency variants would likely be missed in single, block-based, common variant analysis; however, their contribution to disease may be assayed by use of multiblock haplotypes in analysis.

This integrated variation map of the MHC has considerable utility. In the 50 years of study since its first discovery, the MHC has been implicated in almost every human inflammatory and autoimmune disease. Although the MHC has been studied by typing of the classical HLA genes and microsatellites for many years, only rarely has this analysis definitively identified causal variation. Often, association studies using these methods implicate more than one allele at a single locus as influencing disease susceptibility. Although this may represent allelic heterogeneity underlying disease, reinterpreting such results with attention to shared SNP-haplotype variation might point to additional hypotheses regarding the causal variant. For instance, one may find that two different disease-predisposing HLA alleles share a common SNP haplotype, which suggests that a variant carried on that haplotype may, in fact, be the underlying cause of disease. Another common finding in MHC studies is that an extended haplotype, rather than a single variant, is associated with disease. A uniform map of the variation in the region would allow fine mapping of association signals on the basis of rare recombinant chromosomes. Because SNPs are more abundantly present, reliably typed, and cost efficient than microsatellites, they are an excellent choice for this sort of large-scale, high density genotyping. A denser sampling of all the haplotype variation in the region will allow researchers to fully consider all of the 120 genes that lie in the MHC, rather than to focus solely on the classical HLA loci.

The map as shown in FIGS. 1A-1E identifies haplotype blocks covering 24.5% of the MHC. On the basis of the estimated average size of blocks in this region, SNP coverage must be increased four-fold to reach saturation. This map in FIGS. 1A-1E is based on the genotypes only of individuals of European ancestry; variation in other populations must also be examined to unify MHC association results between populations. The additional SNPs to be discovered, as well as genotype information in other populations, may be employed to build a more complete map according to the materials and methods described herein that were employed to build the map as shown in FIGS. 1A-1E.

Ultimately, a full understanding of the patterns of LD and haplotype diversity of this region should allow the identification of a subset of SNPs required for disease studies. This will allow MHC-association studies to be completed cost effectively by using a combination of haplotype-tagging and HLA allele-tagging SNPs. Although a large number of SNPs were used to construct the map as shown in FIGS. 1A-1E, and more SNPs will be needed to fully describe the haplotype structure of the region, an estimate of 10-15 SNPs per locus may be sufficient for common, classical HLA alleles. Moreover, in cases where there is already significant association to a particular locus, these informative SNPs may be used to map outward from the original signal and delimit the region of association. An estimate of a few dozen SNPs may be needed in such endeavors.

SNP-based haplotype approaches will allow the examination of larger disease cohorts and enable the identification of rare recombinant haplotypes that would refine association signals and potentially identify the causal alleles for MHC-associated diseases.

Integrated Map

SNPs used in creating the integrated map as shown in FIG. 1 include the following SNPs, as shown in TABLE 1. All SNPs are located on human chromosome 6, and their respective chromosome positions are shown in Column CHROM_POS. The frequencies of allele types are also shown in Columns FREQ1 and FREQ2. The primer sequences, as well as probe information and flanking sequences for the SNPs are described in detail at: http://www.broad.mit.edu/mpg/idrg/projects/HLA_data/SNP_Info.xls (incorporated herein by reference in its entirety). The primer sequences for SNPs are also provided herein in TABLE 2.

TABLE 1

| SNP | CHROM | CHROM_POS | ALLELE1 | FREQ1 | ALLELE2 | FREQ2 |
|---|---|---|---|---|---|---|
| rs1611750 | 6 | 29891606 | G | 0.187969925 | T | 0.812030075 |
| rs2517862 | 6 | 29898525 | A | 0.192592593 | A | 0.807407407 |
| rs1655930 | 6 | 29942211 | A | 0.821705426 | C | 0.178294574 |
| rs2517706 | 6 | 29963094 | C | 0.602941176 | T | 0.397058824 |
| rs435766 | 6 | 29983284 | A | 0.352941176 | G | 0.647058824 |
| rs410909 | 6 | 29992085 | A | 0.098484848 | C | 0.901515152 |
| rs1264807 | 6 | 30002241 | A | 0.801470588 | C | 0.198529412 |
| rs356963 | 6 | 30013075 | C | 0.925925926 | G | 0.074074074 |
| rs3129012 | 6 | 30032096 | A | 0.189393939 | G | 0.810606061 |
| rs259938 | 6 | 30051799 | C | 0.701492537 | G | 0.298507463 |
| rs2844800 | 6 | 30061564 | A | 0.921875 | G | 0.078125 |
| rs3132129 | 6 | 30071689 | A | 0.073529412 | G | 0.926470588 |
| rs1150736 | 6 | 30098575 | A | 0.294117647 | G | 0.705882353 |
| rs1264709 | 6 | 30112291 | A | 0.801470588 | T | 0.198529412 |
| rs1264701 | 6 | 30122157 | G | 0.860294118 | T | 0.139705882 |
| rs2427749 | 6 | 30163400 | C | 0.820895522 | T | 0.179104478 |
| rs1015465 | 6 | 30170856 | C | 0.110294118 | T | 0.889705882 |
| rs1573297 | 6 | 30200857 | C | 0.694656489 | T | 0.305343511 |
| rs2074477 | 6 | 30216292 | A | 0.111111111 | G | 0.888888889 |
| rs2844786 | 6 | 30228928 | A | 0.333333333 | C | 0.666666667 |
| rs2074473 | 6 | 30238687 | C | 0.485294118 | T | 0.514705882 |
| rs2517614 | 6 | 30248435 | A | 0.169117647 | G | 0.830882353 |
| rs2021722 | 6 | 30258150 | A | 0.167938931 | G | 0.832061069 |
| rs885916 | 6 | 30260107 | A | 0.140740741 | G | 0.859259259 |
| rs928824 | 6 | 30280200 | A | 0.087301587 | G | 0.912698413 |
| rs968909 | 6 | 30300336 | C | 0.838235294 | T | 0.161764706 |
| rs1264626 | 6 | 30303763 | A | 0.825396825 | G | 0.174603175 |
| rs261945 | 6 | 30327954 | C | 0.544776119 | G | 0.455223881 |
| rs3094628 | 6 | 30341046 | C | 0.141791045 | G | 0.858208955 |
| rs1264582 | 6 | 30350326 | A | 0.081481481 | G | 0.918518519 |
| rs1110464 | 6 | 30351625 | A | 0.529411765 | G | 0.470588235 |
| rs1264579 | 6 | 30358950 | C | 0.903703704 | G | 0.096296296 |
| rs3094054 | 6 | 30389245 | G | 0.846153846 | T | 0.153846154 |
| rs3129820 | 6 | 30399307 | A | 0.141791045 | G | 0.858208955 |
| rs970270 | 6 | 30402909 | C | 0.768595041 | T | 0.231404959 |
| rs2187978 | 6 | 30418709 | C | 0.888888889 | T | 0.111111111 |
| rs1264562 | 6 | 30428292 | A | 0.362204724 | C | 0.637795276 |
| rs1150769 | 6 | 30438720 | A | 0.438461538 | G | 0.561538462 |
| rs1264511 | 6 | 30469758 | C | 0.571428571 | G | 0.428571429 |
| rs2524172 | 6 | 30489460 | A | 0.125925926 | G | 0.874074074 |
| rs2021720 | 6 | 30504972 | C | 0.410852713 | G | 0.589147287 |
| rs1059510 | 6 | 30513496 | C | 0.669230769 | T | 0.330769231 |
| rs2844724 | 6 | 30524708 | C | 0.348484848 | T | 0.651515152 |
| rs2516650 | 6 | 30550400 | C | 0.179104478 | G | 0.820895522 |
| rs1362119 | 6 | 30555226 | A | 0.656716418 | T | 0.343283582 |
| rs1468079 | 6 | 30562131 | A | 0.338461538 | C | 0.661538462 |
| rs2516640 | 6 | 30572208 | C | 0.121212121 | T | 0.878787879 |
| rs2074505 | 6 | 30576689 | C | 0.351145038 | T | 0.648854962 |
| rs3130242 | 6 | 30593090 | A | 0.654411765 | G | 0.345588235 |
| rs1264440 | 6 | 30606834 | C | 0.669117647 | T | 0.330882353 |
| rs2252745 | 6 | 30635057 | C | 0.325925926 | T | 0.674074074 |
| rs1059612 | 6 | 30764464 | C | 0.858208955 | T | 0.141791045 |
| rs3129973 | 6 | 30776893 | C | 0.851851852 | T | 0.148148148 |
| rs3130673 | 6 | 30802269 | G | 0.843283582 | T | 0.156716418 |
| rs1264377 | 6 | 30820115 | C | 0.828358209 | T | 0.171641791 |
| rs1264352 | 6 | 30845173 | C | 0.172932331 | G | 0.827067669 |
| rs2535335 | 6 | 30868020 | A | 0.7 | G | 0.3 |
| rs3095354 | 6 | 30891957 | C | 0.544117647 | T | 0.455882353 |
| rs1264332 | 6 | 30901222 | C | 0.715384615 | G | 0.284615385 |
| rs1264314 | 6 | 30926804 | A | 0.782945736 | T | 0.217054264 |
| rs1264297 | 6 | 30940639 | A | 0.705882353 | C | 0.294117647 |
| rs2532936 | 6 | 30950044 | G | 0.298507463 | T | 0.701492537 |
| rs3132571 | 6 | 30961148 | A | 0.632352941 | G | 0.367647059 |
| rs2517434 | 6 | 30999873 | C | 0.92481203 | T | 0.07518797 |
| rs2253417 | 6 | 31025801 | A | 0.088235294 | G | 0.911764706 |
| rs1619376 | 6 | 31039164 | A | 0.213235294 | G | 0.786764706 |
| rs2523897 | 6 | 31049548 | A | 0.15037594 | G | 0.84962406 |
| rs2844670 | 6 | 31061565 | A | 0.792592593 | G | 0.207407407 |
| rs2517523 | 6 | 31082273 | A | 0.635658915 | G | 0.364341085 |
| rs2523882 | 6 | 31097806 | C | 0.75 | T | 0.25 |
| rs2517407 | 6 | 31122306 | C | 0.287878788 | T | 0.712121212 |
| rs1064190 | 6 | 31130758 | G | 0.488549618 | T | 0.511450382 |
| rs1265099 | 6 | 31161025 | A | 0.679389313 | G | 0.320610687 |
| rs1265114 | 6 | 31173035 | C | 0.661764706 | T | 0.338235294 |
| rs915660 | 6 | 31199448 | C | 0.874074074 | G | 0.125925926 |
| rs1265181 | 6 | 31211656 | C | 0.257575758 | G | 0.742424242 |
| rs3132502 | 6 | 31239363 | A | 0.280701754 | G | 0.719298246 |
| rs1793895 | 6 | 31247789 | G | 0.748148148 | T | 0.251851852 |
| rs3134756 | 6 | 31269208 | C | 0.257352941 | T | 0.742647059 |
| rs1793892 | 6 | 31275440 | C | 0.139705882 | T | 0.860294118 |
| rs3130542 | 6 | 31286439 | A | 0.274074074 | G | 0.725925926 |
| rs364415 | 6 | 31327348 | C | 0.80952381 | T | 0.19047619 |
| rs3130690 | 6 | 31340260 | G | 0.832061069 | T | 0.167938931 |
| rs2524227 | 6 | 31356125 | A | 0.353383459 | G | 0.646616541 |
| rs2854008 | 6 | 31366306 | A | 0.291044776 | G | 0.708955224 |
| rs2853996 | 6 | 31386696 | C | 0.821705426 | T | 0.178294574 |
| hCV2995747 | 6 | 31393338 | A | 0.777777778 | G | 0.222222222 |
| rs2301747 | 6 | 31425383 | C | 0.902985075 | G | 0.097014925 |
| rs2256184 | 6 | 31434180 | A | 0.612403101 | G | 0.387596899 |
| hCV2995705 | 6 | 31441607 | C | 0.066176471 | G | 0.933823529 |
| rs2855804 | 6 | 31521290 | C | 0.681481481 | T | 0.318518519 |
| rs3132464 | 6 | 31531399 | C | 0.259259259 | T | 0.740740741 |
| rs2516400 | 6 | 31553393 | C | 0.671641791 | T | 0.328358209 |
| hCV3273612 | 6 | 31564553 | A | 0.674242424 | T | 0.325757576 |
| rs361525 | 6 | 31606263 | A | 0.068181818 | G | 0.931818182 |
| rs986475 | 6 | 31619673 | C | 0.066666667 | T | 0.933333333 |
| rs2857595 | 6 | 31631860 | A | 0.161764706 | G | 0.838235294 |
| rs2844476 | 6 | 31645038 | A | 0.378787879 | G | 0.621212121 |
| rs750332 | 6 | 31670185 | C | 0.233082707 | T | 0.766917293 |
| rs1052486 | 6 | 31673871 | C | 0.465648855 | T | 0.534351145 |
| rs3117583 | 6 | 31682962 | A | 0.857142857 | G | 0.142857143 |
| rs805256 | 6 | 31698902 | A | 0.803149606 | G | 0.196850394 |
| rs805290 | 6 | 31711788 | C | 0.735294118 | T | 0.264705882 |
| rs805281 | 6 | 31724677 | A | 0.736842105 | G | 0.263157895 |
| rs805292 | 6 | 31753147 | A | 0.21641791 | G | 0.78358209 |
| rs1150793 | 6 | 31789133 | A | 0.961538462 | G | 0.038461538 |
| rs707928 | 6 | 31814030 | A | 0.681481481 | G | 0.318518519 |
| rs480092 | 6 | 31836340 | C | 0.174242424 | T | 0.825757576 |
| rs2075799 | 6 | 31850226 | C | 0.941176471 | T | 0.058823529 |
| rs539689 | 6 | 31857089 | C | 0.492647059 | G | 0.507352941 |
| rs2763979 | 6 | 31866094 | C | 0.653846154 | T | 0.346153846 |
| rs3130679 | 6 | 31879245 | A | 0.904 | G | 0.096 |
| rs574914 | 6 | 31890829 | A | 0.151515152 | G | 0.848484848 |
| rs660550 | 6 | 31909117 | A | 0.52238806 | C | 0.47761194 |
| rs605203 | 6 | 31918651 | G | 0.351145038 | T | 0.648854962 |
| rs589428 | 6 | 31919809 | G | 0.62962963 | T | 0.37037037 |
| rs558702 | 6 | 31941915 | A | 0.082706767 | G | 0.917293233 |
| rs419788 | 6 | 31990590 | C | 0.705882353 | T | 0.294117647 |
| rs429608 | 6 | 31992054 | A | 0.112781955 | G | 0.887218045 |
| rs433061 | 6 | 32043622 | A | 0.090225564 | G | 0.909774436 |
| rs1269852 | 6 | 32119789 | C | 0.080882353 | G | 0.919117647 |
| rs2269425 | 6 | 32150480 | C | 0.823076923 | T | 0.176923077 |
| rs204989 | 6 | 32188886 | A | 0.110294118 | G | 0.889705882 |
| rs2071280 | 6 | 32191654 | C | 0.294117647 | G | 0.705882353 |
| rs2071277 | 6 | 32210496 | C | 0.435114504 | T | 0.564885496 |
| rs3130316 | 6 | 32250273 | C | 0.691588785 | T | 0.308411215 |
| rs926070 | 6 | 32283275 | C | 0.346153846 | T | 0.653846154 |
| rs742697 | 6 | 32318423 | C | 0.345864662 | T | 0.654135338 |
| rs3129960 | 6 | 32327712 | A | 0.227941176 | G | 0.772058824 |
| rs2022534 | 6 | 32333790 | A | 0.596899225 | G | 0.403100775 |
| rs3129907 | 6 | 32350292 | A | 0.759398496 | G | 0.240601504 |
| rs2143462 | 6 | 32361583 | C | 0.860294118 | T | 0.139705882 |
| rs1555115 | 6 | 32381050 | C | 0.895522388 | G | 0.104477612 |
| rs2395158 | 6 | 32401103 | A | 0.880597015 | G | 0.119402985 |
| rs2395161 | 6 | 32414066 | A | 0.867647059 | C | 0.132352941 |
| rs983561 | 6 | 32430210 | A | 0.785185185 | C | 0.214814815 |
| rs2239802 | 6 | 32438402 | C | 0.723880597 | G | 0.276119403 |
| rs7194 | 6 | 32439002 | A | 0.563492063 | G | 0.436507937 |
| rs1987529 | 6 | 32502240 | A | 0.85 | G | 0.15 |
| rs1059544 | 6 | 32578551 | C | 0.268292683 | T | 0.731707317 |
| rs2858860 | 6 | 32598186 | G | 0.454545455 | T | 0.545454545 |
| rs2395253 | 6 | 32717006 | A | 0.053030303 | G | 0.946969697 |
| rs2857210 | 6 | 32738722 | A | 0.35483871 | G | 0.64516129 |
| rs719654 | 6 | 32749097 | A | 0.22962963 | G | 0.77037037 |
| rs2157080 | 6 | 32758398 | A | 0.376923077 | G | 0.623076923 |
| rs2621343 | 6 | 32771751 | C | 0.383458647 | T | 0.616541353 |
| rs1383267 | 6 | 32830393 | C | 0.560606061 | T | 0.439393939 |
| rs1029295 | 6 | 32853431 | C | 0.105263158 | T | 0.894736842 |
| rs241404 | 6 | 32862944 | C | 0.428571429 | T | 0.571428571 |
| rs2187688 | 6 | 32868648 | A | 0.446153846 | G | 0.553846154 |
| rs151719 | 6 | 32900606 | A | 0.785714286 | G | 0.214285714 |
| rs188245 | 6 | 32955171 | C | 0.529411765 | T | 0.470588235 |
| rs663310 | 6 | 33009016 | C | 0.217054264 | T | 0.782945736 |
| rs2071351 | 6 | 33045675 | A | 0.84496124 | G | 0.15503876 |
| rs2144014 | 6 | 33067793 | C | 0.703125 | T | 0.296875 |
| rs3130216 | 6 | 33079418 | A | 0.574626866 | G | 0.425373134 |
| rs3129272 | 6 | 33099767 | C | 0.696296296 | T | 0.303703704 |
| rs2294478 | 6 | 33102058 | A | 0.474074074 | C | 0.525925926 |
| rs734181 | 6 | 33133246 | C | 0.201550388 | G | 0.798449612 |
| rs2076311 | 6 | 33148710 | A | 0.298507463 | C | 0.701492537 |
| rs2855433 | 6 | 33161160 | A | 0.701492537 | C | 0.298507463 |
| rs421446 | 6 | 33178124 | A | 0.729323308 | G | 0.270676692 |
| rs213213 | 6 | 33186822 | C | 0.725925926 | T | 0.274074074 |
| rs213194 | 6 | 33198941 | A | 0.234375 | G | 0.765625 |
| rs105445 | 6 | 33220331 | C | 0.146153846 | G | 0.853846154 |
| rs464865 | 6 | 33259080 | A | 0.441176471 | G | 0.558823529 |
| rs1014779 | 6 | 33278611 | A | 0.533333333 | G | 0.466666667 |
| rs1061783 | 6 | 33284571 | A | 0.544776119 | G | 0.455223881 |
| rs3130267 | 6 | 33318929 | G | 0.5390625 | T | 0.4609375 |
| rs456993 | 6 | 33360326 | C | 0.444444444 | T | 0.555555556 |
| rs211457 | 6 | 33367887 | C | 0.873134328 | T | 0.126865672 |
| rs1705003 | 6 | 33388001 | A | 0.888888889 | G | 0.111111111 |
| rs2076775 | 6 | 33396500 | C | 0.634328358 | G | 0.365671642 |
| rs453590 | 6 | 33405670 | C | 0.634328358 | T | 0.365671642 |
| rs1755047 | 6 | 33433266 | C | 0.451851852 | G | 0.548148148 |
| rs210190 | 6 | 33466198 | A | 0.066176471 | G | 0.933823529 |
| rs1755038 | 6 | 33467280 | A | 0.909774436 | G | 0.090225564 |
| rs769051 | 6 | 33476004 | G | 0.659090909 | T | 0.340909091 |
| rs210180 | 6 | 33487120 | A | 0.348148148 | T | 0.651851852 |
| rs210196 | 6 | 33509584 | A | 0.669117647 | G | 0.330882353 |
| rs210203 | 6 | 33513090 | A | 0.37037037 | G | 0.62962963 |
| rs210132 | 6 | 33538781 | G | 0.536764706 | T | 0.463235294 |
| rs210135 | 6 | 33542803 | A | 0.827868852 | T | 0.172131148 |
| rs210139 | 6 | 33545520 | A | 0.564885496 | C | 0.435114504 |
| rs210145 | 6 | 33549551 | C | 0.467213115 | G | 0.532786885 |
| rs396746 | 6 | 33558906 | A | 0.148148148 | C | 0.851851852 |
| rs210120 | 6 | 33576523 | A | 0.588235294 | G | 0.411764706 |
| rs407415 | 6 | 33581077 | A | 0.786764706 | G | 0.213235294 |
| rs999943 | 6 | 33626118 | C | 0.266666667 | T | 0.733333333 |
| rs2229634 | 6 | 33640290 | C | 0.701492537 | T | 0.298507463 |
| rs658087 | 6 | 33667130 | A | 0.148148148 | T | 0.851851852 |
| rs2281829 | 6 | 33677752 | A | 0.544117647 | G | 0.455882353 |
| rs1555965 | 6 | 33679261 | A | 0.555555556 | G | 0.444444444 |
| rs549652 | 6 | 33688213 | A | 0.147058824 | G | 0.852941176 |
| rs608971 | 6 | 33703990 | C | 0.78030303 | T | 0.21969697 |
| rs530614 | 6 | 33716891 | A | 0.161764706 | G | 0.838235294 |
| rs2395449 | 6 | 33730616 | A | 0.388059701 | T | 0.611940299 |
| rs943473 | 6 | 33745761 | C | 0.816176471 | G | 0.183823529 |
| rs2395402 | 6 | 33755534 | A | 0.559701493 | G | 0.440298507 |
| rs2894342 | 6 | 33776504 | A | 0.25 | C | 0.75 |
| rs1547668 | 6 | 33777490 | A | 0.139344262 | G | 0.860655738 |

TABLE 2

| | | | |
|---|---|---|---|
| snp | HG12 | Primer 1 (SEQ ID NOS: 1-639) | Primer 2 (SEQ ID NOS: 640-1278) |

| | | | |
|---|---|---|---|
| rs1611750 | 29891606 | ACGTTGGATGTGAGGACCACAAAAGTCAGG | ACGTTGGATGCCCATCAATTGACCCAGTTC |
| rs2517862 | 29898525 | ACGTTGGATGGGGAAAACAGCAAGGTACAG | ACGTTGGATGTGTTCTTTCTCCCTTTGCAC |
| rs885933 | 29913724 | ACGTTGGATGTGTAGCCAGTCATAGCTGTC | ACGTTGGATGACTTCTCAGCTGCATCGATG |
| rs886399 | 29913789 | ACGTTGGATGGCTATCTGCCCTTTTGCTAC | ACGTTGGATGTGGCTACATTTGACACCCTC |
| rs2394233 | 29924799 | ACGTTGGATGGATAAATGGGTGTTGTTTCG | ACGTTGGATGGCAAAACACGGAAAAAGTTC |
| rs1054175 | 29925728 | ACGTTGGATGGGCCCCATGATGTATAAATG | ACGTTGGATGACAGGTACACTGCAAAAGTG |
| rs1611545 | 29931342 | ACGTTGGATGACAATACCTGCAGTACCCTC | ACGTTGGATGAAAACTTCCCTCATCCCAGC |
| rs1632910 | 29935874 | ACGTTGGATGGGCTCAACAGACTCGGAATG | ACGTTGGATGACGTGAGCATATGAGGGCAT |
| rs2517762 | 29938895 | ACGTTGGATGTCCCTGGAATACTGATGAGG | ACGTTGGATGAAAGCAGAGAACAAGGCCTG |
| rs1655930 | 29942211 | ACGTTGGATGTGTAGTAATCCTAGTGCTGG | ACGTTGGATGATGGGTCCAATTTTCCACCC |
| rs2905764 | 29953186 | ACGTTGGATGTTTGGTGCCAGAGAGTAAGC | ACGTTGGATGTTCTGTCTCATGCACTCAGG |
| rs1616549 | 29957706 | ACGTTGGATGAGTTCACGTGGACATCCATG | ACGTTGGATGTTTGTGCTGAAGTGTGCAGG |
| rs376253 | 29957969 | ACGTTGGATGGGGTTATGGTGCATACGTTC | ACGTTGGATGTCACTCCAGGACTCAGGTTC |
| rs1961135 | 29958142 | ACGTTGGATGGAACCCTCCTTTTCAGTGAC | ACGTTGGATGGGCTGATACTCTGGGTTATC |
| rs2735099 | 29958264 | ACGTTGGATGGTCAGAAAAGATGGGCAGAC | ACGTTGGATGTGCTCCTCAATTCCACATGC |
| rs2524037 | 29958386 | ACGTTGGATGATAGGCTCCTTTGCAGAAGG | ACGTTGGATGAAGAACCTTGGGACACGATG |
| rs2517706 | 29963094 | ACGTTGGATGGGATTAGAAGCATGAGCCAC | ACGTTGGATGGGCACACAAGGTGCATTTTG |
| rs2975041 | 29964111 | ACGTTGGATGCTCCATTCTCTGTCTCAAAG | ACGTTGGATGCTTGTATCTGACTGATTTTC |
| rs382875 | 29967813 | ACGTTGGATGAGTCTTTGAGGGAAAGGAGG | ACGTTGGATGAAAATTCCTGGTGCCCAAGG |
| rs2517701 | 29969404 | ACGTTGGATGATTGGAGTCATGGGAACCTG | ACGTTGGATGACCCTAGGTAAGAGGATGTG |
| rs2517699 | 29970029 | ACGTTGGATGCCCCACTTCTCACATGATAC | ACGTTGGATGGCCTCTGTCTTCTCTTTCTG |
| rs435766 | 29983284 | ACGTTGGATGTCCATGCCTTTCTGTGTGGG | ACGTTGGATGTGAGGAATAGGGGTCAGCAG |
| rs410909 | 29992085 | ACGTTGGATGCACACAGATTCACACACACG | ACGTTGGATGAAGTCAGCCTGTCCCACAAC |
| rs2246555 | 29992480 | ACGTTGGATGATCTCCCCCCTTCTTCAGAG | ACGTTGGATGGTCTCTTTTTCCTGGAGGTG |
| rs2394255 | 29993239 | ACGTTGGATGGGTGTAAAGGAAACTGCAGG | ACGTTGGATGATGGAGACAGTCCTTTCCAG |
| rs1264807 | 30002241 | ACGTTGGATGTAACATTCCCCTTTCTCCAC | ACGTTGGATGGAGATATATCTTACCCTAACC |
| rs1632926 | 30004710 | ACGTTGGATGGCACAATTCTCATCCGACAC | ACGTTGGATGTAACCTCTGTCTCCTTCCAG |
| rs2530388 | 30010564 | ACGTTGGATGCCCAACTCTCAACAAGGTAG | ACGTTGGATGTCAGCCTCGTTATTCCTTCC |
| rs356962 | 30012120 | ACGTTGGATGAACACAGAAGGCAGAGGTTG | ACGTTGGATGAAAGTCTTGCTCTTGTCCCC |
| rs356963 | 30013075 | ACGTTGGATGTGTGGTTGCTCCATTCATGC | ACGTTGGATGCAGACAAATGGCAGTTAGCC |
| rs2286405 | 30016829 | ACGTTGGATGAAAACAGGCAGTGCATGAGC | ACGTTGGATGTCACCTCAAAGTTGCAAGCG |
| rs2240619 | 30018890 | ACGTTGGATGATTCCTCTCCGTCAGGACAG | ACGTTGGATGATCTCCTGTAGATCTCCCGG |
| rs886997 | 30021056 | ACGTTGGATGACAAGGTTCTACTGAAGGGC | ACGTTGGATGACCATGGGCTTTATGTGGTC |
| rs3129012 | 30032096 | ACGTTGGATGCCCACTTGGCATGGTGAATC | ACGTTGGATGAAGGTCTTAGGAGAGGGCTG |
| rs1150743 | 30035516 | ACGTTGGATGCTGGACATTTCATCAGGACC | ACGTTGGATGATCTCAGCATGTGAGGCTTC |
| rs259938 | 30051799 | ACGTTGGATGGAGGCTATGGTACCAAACTG | ACGTTGGATGCTGTGGATTCTGGGATAGAG |
| rs2844800 | 30061564 | ACGTTGGATGCTCGGACTCCTTTGCTCATT | ACGTTGGATGCCCACAGGAAAGGAGAAAAG |
| rs259948 | 30065078 | ACGTTGGATGACTTTCACTCCACTGCCTTC | ACGTTGGATGAAACTTTCGTGCTGCAGGTG |
| rs3132129 | 30071689 | ACGTTGGATGCGTCTCCCTTTGTAAGACAG | ACGTTGGATGACTGCTGAAGAGTGACAAGC |
| rs1150736 | 30098575 | ACGTTGGATGTCGCGGAGTTGTTGGTGGAAG | ACGTTGGATGTAAACTTCCACAGGGCCTCC |
| rs1264709 | 30112291 | ACGTTGGATGTTTTGGAGCTAGGATTCTGG | ACGTTGGATGCTACCTCACTTCTGCATTTC |
| rs1264701 | 30122157 | ACGTTGGATGCCCTCTATGCTCACTATCTC | ACGTTGGATGAAAAGAGCCAAGGGCAACAC |
| rs2023472 | 30160044 | ACGTTGGATGATTTTTTCAGGCTCCCTGTG | ACGTTGGATGTCAGTTTCTCAACCCAACCC |
| rs2427749 | 30163400 | ACGTTGGATGAGTGGGGTCACAATGTCTTC | ACGTTGGATGTATTGTTTGAGCCTGGGAGG |
| rs1015465 | 30170856 | ACGTTGGATGGATAGTGCCCATTCACACTG | ACGTTGGATGAATGTCCAGAGCTGATGAGG |
| rs1419673 | 30181231 | ACGTTGGATGAGTCATTGGCCTGTTTTTCG | ACGTTGGATGTGACATCTACAAACAGTTTC |
| rs1362104 | 30186130 | ACGTTGGATGGGGAAAAAAAACCGTAAGTG | ACGTTGGATGGGCAGTTGTAAATATTTTTC |
| rs1573297 | 30200857 | ACGTTGGATGAGTCTTGTGGCTGCAAGAAC | ACGTTGGATGGGGAAGAATCTGCTTCCAAG |
| rs2285797 | 30204577 | ACGTTGGATGATCCAAAGCACCCAAACCTG | ACGTTGGATGCAAAAGACACAGCTAAAGCC |
| rs2074477 | 30216292 | ACGTTGGATGTTCTTTGCACTCCACCTCTG | ACGTTGGATGTGGATGTGTGGTAGTTCCTG |
| rs2844786 | 30228928 | ACGTTGGATGTAGCCAAAGAAATCCTGAGC | ACGTTGGATGCCTGACAAAAATGTCTCTAG |
| rs2074473 | 30238687 | ACGTTGGATGACTTCCACTTCCCAGTAGAC | ACGTTGGATGTGTACAAGAGTGCCTACCTG |
| rs2517614 | 30248435 | ACGTTGGATGCATAAAAAGCTTCCACCAGC | ACGTTGGATGTCATCACTGCCATCACAAGC |
| rs2021722 | 30258150 | ACGTTGGATGACACTTGGCTTACTTTCCCC | ACGTTGGATGAGCCCTGGTAGTTTTTGTGG |
| rs885916 | 30260107 | ACGTTGGATGTGGATTTTTCTTCCCCACTC | ACGTTGGATGTAAGATGTTGCCACAGTTCC |
| rs3129696 | 30270016 | ACGTTGGATGAACTCCTGACGTGATCTGCC | ACGTTGGATGAAAAAATAGGCTGGGCACGG |
| rs928824 | 30280200 | ACGTTGGATGCGCAAAAAAAAGTTGCAGTC | ACGTTGGATGGGAATTGTTGGGTGATATGG |
| rs3132656 | 30289505 | ACGTTGGATGACCATGATTCTGAGGCCTCC | ACGTTGGATGCTGCCGATAAAGACATACCC |
| rs968909 | 30300336 | ACGTTGGATGCAGTGTGAAATTGGACCCTG | ACGTTGGATGTCCCTAAAGGGATCAATGGC |
| rs1264626 | 30303763 | ACGTTGGATGAGTCATGAAAGATCCACCCC | ACGTTGGATGCCCACCCAAATTTCGTGTTC |
| rs261956 | 30320688 | ACGTTGGATGTTAGCAGGTATGGTGGCATG | ACGTTGGATGAACTCCTGGCTCAAATGATC |
| rs261945 | 30327954 | ACGTTGGATGCATGGCCTCTTATGAGAACC | ACGTTGGATGGGGCAACAAGAGTGAAACTG |
| rs3094628 | 30341046 | ACGTTGGATGAGGTGTGTTGGAAGGTGGTG | ACGTTGGATGCCCATGCATGCAATTACCTC |
| rs1264582 | 30350326 | ACGTTGGATGTTGATTCCCCCTGCTGCTTC | ACGTTGGATGTCGTGTCAGTGGAAGCTGGG |
| rs1110464 | 30351625 | ACGTTGGATGCTCAGAACTGCTGAAAACTG | ACGTTGGATGAGACTCGTTGCTCTCTTTTC |
| rs1264579 | 30358950 | ACGTTGGATGTCTGCCTTCTTTGCTCAAGC | ACGTTGGATGATTAGCTGAGTCTGGTGGTG |
| rs984801 | 30376931 | ACGTTGGATGGCAAGTAGCAGGAAATTCAG | ACGTTGGATGCCTCTGGAAGATAAAATGGG |
| rs3094054 | 30389245 | ACGTTGGATGCCCAGTGGCAAATCAATTAC | ACGTTGGATGAATAACCCCTGGCTCAGAAC |
| rs3129820 | 30399307 | ACGTTGGATGCCTCCTAGTTTCTGCTTTCC | ACGTTGGATGCTCACAGAAGAGAGGATGAG |
| rs970270 | 30402909 | ACGTTGGATGTCAAAGGACTGCAGGAACAG | ACGTTGGATGGCTGCAAATACATGTGTGGG |
| rs2187978 | 30418709 | ACGTTGGATGAAATGTCAGAGTGGTGTGGG | ACGTTGGATGTGAGTGGGATTGAAAAGCTG |
| rs1264562 | 30428292 | ACGTTGGATGTGTCGCCTCCTGCACTTCAT | ACGTTGGATGTCCAACAGACGCTTTTCTGG |
| rs1150769 | 30438720 | ACGTTGGATGCCAGCAGTTCATTCCTGAAC | ACGTTGGATGTGGGCTGAGTTCCTCACTTG |
| rs1264534 | 30449506 | ACGTTGGATGTTCTTCTCTCTCTTCTTCTC | ACGTTGGATGCGTTAATGAATCTAGGAGCTG |
| rs1264525 | 30459721 | ACGTTGGATGGGAAACTATCACAAGGACAG | ACGTTGGATGCTCATTGTTCAGTTTCCACC |
| rs1264511 | 30469758 | ACGTTGGATGTATGATTCCCCTCCTCCTTC | ACGTTGGATGGAACTATGATCCTGACCCTG |
| rs2187975 | 30480842 | ACGTTGGATGTGCCATGATGGTAAGCTTCC | ACGTTGGATGTCTGCAGGCTTTATGGGAAG |
| rs2524172 | 30489460 | ACGTTGGATGACTTCCCTTTCTTAGCCACC | ACGTTGGATGCACTGGGAGAGATGAGTATG |
| rs3131112 | 30503440 | ACGTTGGATGCCTGGAGCTTTATAGTAAGTC | ACGTTGGATGGAAGGACTTTGAATATCCAC |
| rs2021720 | 30504972 | ACGTTGGATGAGGTCCTTGATTCTGGACTC | ACGTTGGATGTTTCGCGCTGGGGAGCCTCT |
| rs2021719 | 30505249 | ACGTTGGATGAGAATCCTGGACTCTCAAGG | ACGTTGGATGATTTGTCCCCAATCCATCGG |
| rs1059510 | 30513496 | ACGTTGGATGGGGACACCGCACAGATTTTC | ACGTTGGATGCCTCGCTCTGATTGTAGTAG |
| rs2516665 | 30523713 | ACGTTGGATGTTGAGACCATCCTGGCTAAC | ACGTTGGATGCCACCACGCCCAGAAAATTT |
| rs2844724 | 30524708 | ACGTTGGATGGTGAGATCAAGAGTACTATTC | ACGTTGGATGCTGTGTCACTTGGTAAGTAG |
| rs2023608 | 30531495 | ACGTTGGATGGGTTGTGCTAACCTGACTTC | ACGTTGGATGGTGGGAAGGATTCCACAAAG |
| rs2534805 | 30541820 | | |
| rs2516650 | 30550400 | ACGTTGGATGTAGGTATACCTGTGCCATGG | ACGTTGGATGGGAAGGGATAGCATTAGGAG |
| rs1362119 | 30555226 | ACGTTGGATGCAGTGCCTTGATACCTGAAC | ACGTTGGATGACAGCCTGGATGGCTTATAG |
| rs1468079 | 30562131 | ACGTTGGATGAGACATCATGACCATTCACC | ACGTTGGATGAGGCATTCAAATTGGAGAAG |
| rs2524222 | 30566921 | ACGTTGGATGCCAAGTGGTAAGTGAGATAG | ACGTTGGATGTCTCCCAGAACTTATCACAC |
| rs2516640 | 30572208 | ACGTTGGATGCCCAACCCTGTAAAATCCAG | ACGTTGGATGTTCTTAGCCACAGTCAGCTG |
| rs2074505 | 30576689 | ACGTTGGATGCAAGTTGCCCTCTCTCATTG | ACGTTGGATGTTTCACCTCTTTTCCTCGGG |
| rs3130242 | 30593090 | ACGTTGGATGTCACCTTTCCCACAACTCTG | ACGTTGGATGACAAACAGGAAGGAGGCAAG |
| rs1264444 | 30603213 | ACGTTGGATGATGTTGACCAGGATGGTCTC | ACGTTGGATGTAATCCCAGCACTTTGGGAG |
| rs1264440 | 30606834 | ACGTTGGATGAATTCTCCCTTTGGGACAGG | ACGTTGGATGGGGATTATGCTGGAGGTAGC |
| rs1264437 | 30609619 | ACGTTGGATGACGCTGGCCTACATTTCAAG | ACGTTGGATGTTTTCCTGGAGAGGAAGAGG |
| rs1264424 | 30624639 | ACGTTGGATGGAACTCTGACACAGGATCAG | ACGTTGGATGCCACCCCATGAGGAATAATG |
| rs1264423 | 30627013 | ACGTTGGATGCGGTGCATCTTTCATATGAG | ACGTTGGATGCCATGGAACACTCCTGAAAG |
| rs2252745 | 30635057 | ACGTTGGATGTTGGGAGGACAAAAAGGCTG | ACGTTGGATGTGTGCCGGAAAAAACACAGG |
| rs3132607 | 30645288 | ACGTTGGATGAGGAGTTTGAGACCAGCCTG | ACGTTGGATGTGAGAAGCTGGGACTACAGG |
| rs2394388 | 30654985 | ACGTTGGATGGAGGAGTATGGTAGGAGATG | ACGTTGGATGAAGCCAGTCTTTGCAGTAGC |
| rs3132608 | 30665408 | ACGTTGGATGGCACCCACTGACAGTAAGAG | ACGTTGGATGTAGGGAGAAAGATCGAAGGG |
| rs1127955 | 30676469 | ACGTTGGATGAAACAGAACCTGACACCAGC | ACGTTGGATGTCCCAAATGTTCCCACAAGC |
| rs1124795 | 30677163 | ACGTTGGATGTCCTGACCCCTATCATCCTG | ACGTTGGATGTATGCTCTGGGAGCCCTCAAC |
| rs1076829 | 30682989 | ACGTTGGATGCAGGCACACAGCTTTTTCAC | ACGTTGGATGTCAGTTGGAGAAACCCACAC |
| rs1076828 | 30684025 | ACGTTGGATGTGATCTGCAACCTATCCCAG | ACGTTGGATGTATGGCTAACTTGTCCTGGC |
| rs2285320 | 30696449 | ACGTTGGATGATGGCGACTCACGCTCCCTG | ACGTTGGATGTAGAGGTCCCAAGGTAGCTG |
| rs2394392 | 30705580 | ACGTTGGATGAGAGTTCCTCTGACCCAGAC | ACGTTGGATGTTGCAGCAGAGCTGGGACAAG |
| rs2239888 | 30705674 | ACGTTGGATGCCTCTGTACTTTATTTTCTAC ACGTTGGATGTGAGGAGACAGGCAGGGTAG | |
| rs1075496 | 30714001 | ACGTTGGATGCCATGCTTTTTGCAACTGCC | ACGTTGGATGTTCCATCCCTAGTTTCTGCC |
| rs3130644 | 30716427 | ACGTTGGATGAAGTGCTGGGATTACAGCTG | ACGTTGGATGCAGACAGCAGGTATGGTAAG |
| rs3094090 | 30725716 | ACGTTGGATGACCTGTAGTCCCAGCTACTC | ACGTTGGATGTCTCGGCTCACTACAATCTC |
| rs2239886 | 30726450 | ACGTTGGATGGCTCTCTCTAAATGCTAGGC | ACGTTGGATGAGCAGTCAGCATCAAAGCTC |
| rs2394394 | 30733626 | ACGTTGGATGACCTGAGATCGGGAGCTTGA | ACGTTGGATGTTACAGGCATGCACCACCAC |
| rs2075015 | 30736108 | ACGTTGGATGAGCTTGGCTTTTCTCCAGAG | ACGTTGGATGTCCATGGAGTAGGTACAAGG |
| rs25525 | 30746293 | ACGTTGGATGATCCCCTTTGGGTGAATCTG | ACGTTGGATGAGACTTGTCATTCCAGGTCC |
| rs2244011 | 30750829 | ACGTTGGATGCAGACTGTTTGAGCCTGTTG | ACGTTGGATGAAGTTGAAAACCTCCAGCCC |
| rs1059612 | 30764464 | ACGTTGGATGCCCCCCTCATTTTGACATCC | ACGTTGGATGTCATGGCCCACATGACTGTG |
| rs3129973 | 30776893 | ACGTTGGATGAGTTCCCAACCCAAATCCAG | ACGTTGGATGGATGCACAACATCAAGAAGC |
| rs2894045 | 30788522 | ACGTTGGATGGGGCACCTTGAAAAAAGAGC | ACGTTGGATGAAATATGGCTCTGTTCCGCC |
| rs2394402 | 30789242 | ACGTTGGATGTTTCTGCAACCTCTGCCTCC | ACGTTGGATGTTTGTGGCATGCGCCTGTAG |
| rs3130673 | 30802269 | ACGTTGGATGTCTTTAAGTGGATGGGCTCG | ACGTTGGATGTGGCAGGCAGAGCAATTTAG |
| rs3131041 | 30810816 | ACGTTGGATGAGGTTGAAGCGATTCTCCTG | ACGTTGGATGACAAAAGTTAGCTGGGCGTG |
| rs1264377 | 30820115 | ACGTTGGATGAAGACCACTTCAGAGTCCAG | ACGTTGGATGGGAGAGGTGGTCATGATCAG |
| rs2394403 | 30823632 | ACGTTGGATGCTATTCCAAAACATCACTGGC | ACGTTGGATGCGGCCTATTTCTAGTCTTTTG |
| rs1264364 | 30831067 | ACGTTGGATGAGCCTCCCACCCACTCAAAG | ACGTTGGATGTTGGGTGGTCGATGGGACTG |
| rs2894046 | 30837877 | ACGTTGGATGCCATGGTTGAAGGAGAAGAG | ACGTTGGATGATCTTCTGTGGCAGACGTAG |
| rs1264352 | 30845173 | ACGTTGGATGCTTGGTACAAGTGAAACTGG | ACGTTGGATGGCTCTTGCTCTTTCTTCTGG |
| rs915664 | 30850194 | ACGTTGGATGTATGACAGCACGTTTCTGCC | ACGTTGGATGCCTCAAGGAGGCAGTTAAAC |
| rs2535338 | 30860692 | ACGTTGGATGGCCTGGCAACATAGCAAGAC | ACGTTGGATGTCAGCCTCATGAGTAGCTGG |
| rs2535335 | 30868020 | ACGTTGGATGACCCCTCATCTCCTAAGCTC | ACGTTGGATGTGAGCTGTCTTCCTTGCCTC |
| rs2250264 | 30876536 | ACGTTGGATGAGGAGGGAAGGAAGTATAAC | ACGTTGGATGGAAACTGTCACCACAATCAAG |
| rs3095354 | 30891957 | ACGTTGGATGGCTGCATAATAAATTGCCCC | ACGTTGGATGGTGTGTATGTGTTTAAGAGAG |
| rs1264332 | 30901222 | ACGTTGGATGGGAAAGAGATTCAGGCTTGG | ACGTTGGATGCCTTTCTGACCTCTCTCTTG |
| rs2855542 | 30912003 | ACGTTGGATGGAAACTAGGGCAGAGATCAG | ACGTTGGATGTCTAAGCCGTTGTTTATGGG |
| rs3130799 | 30921946 | ACGTTGGATGTGTGACTGATGGAGACCAGG | ACGTTGGATGTGCATCCTCATGGTGAGCAG |
| rs1264314 | 30926804 | ACGTTGGATGCTCCAAAAGAGGTGTGCCTA | ACGTTGGATGCCAGACTGGGCAACAAAATG |
| rs1264297 | 30940639 | ACGTTGGATGTCTAAGAGCCACTTCTCAGC | ACGTTGGATGTGTTTAGGGATCTGTGTGGG |
| rs2532936 | 30950044 | ACGTTGGATGAAAGAGCCTGCAAAAGCCAG | ACGTTGGATGTAGTCATGGGTAGGGTATGG |
| rs3132571 | 30961148 | ACGTTGGATGTTGCCTAGAGCTGAGTTGAG | ACGTTGGATGTCAGTGGCCGAGAAAAACAC |
| rs2240804 | 30976671 | ACGTTGGATGAAAGGGGCAGAGCATGGAAG | ACGTTGGATGATCTTGGCATGGGCCAGATC |
| rs2530715 | 30989357 | ACGTTGGATGTTGGAGGTTGTTGTGGGCAC | ACGTTGGATGGGCCTTTGAGGCCACATCAA |
| rs2517441 | 30998212 | ACGTTGGATGCAAGACTGCATACAGGAATAC | ACGTTGGATGCCATCCTGGTCTTAATCTTC |
| rs2517434 | 30999873 | ACGTTGGATGATCACCGGAAAGACCAAAGC | ACGTTGGATGGATTAAACCATGGCCACTGG |
| rs2523927 | 31009436 | ACGTTGGATGAAACTTGGGCCAGTGTCAAC | ACGTTGGATGATCGAGCCATTGCACTCCAG |
| rs2253417 | 31025801 | ACGTTGGATGAAACCTTCCCCCAAAGACTG | ACGTTGGATGCAACATGGCAGATTAGCATC |
| rs1619376 | 31039164 | ACGTTGGATGAAGAGAAAAATGGGCCCAGC | ACGTTGGATGTGAGTCAAATGTGAGGGTGG |
| rs1632866 | 31047876 | ACGTTGGATGGGGTTCTTTGTGTTATACTTG | ACGTTGGATGCCCACTGGAATAACATACTC |
| rs2523897 | 31049548 | ACGTTGGATGCAACTGCAGACTCCAAGGTG | ACGTTGGATGTGGTGTTAGAGCCTGCAGC |
| rs2844670 | 31061565 | ACGTTGGATGTGCCTCTTACTTGTGCCTTG | ACGTTGGATGCACCTCCTTGAATGGAAGTG |
| rs2252195 | 31075647 | ACGTTGGATGAACTTCTTAGCTTCTATAAT | ACGTTGGATGCTTTGTTTTAGAATTTTTAAAAC |
| rs2517523 | 31082273 | ACGTTGGATGGAGAGGTCACTAGCATTAGC | ACGTTGGATGGCCTTTTGAGCCATCTCTTG |
| rs2523882 | 31097806 | ACGTTGGATGAAGACAGAGGTGAGGAATGC | ACGTTGGATGTAAAACACAGCCTCCTTGGG |
| rs3130959 | 31112196 | ACGTTGGATGGGCCAAATTGACTTTTCACC | ACGTTGGATGAATCTGGTTTGCCAGCACAG |
| rs2517407 | 31122306 | ACGTTGGATGCCATGTTCAACCTTTGGAGG | ACGTTGGATGGCTGTTGGACAGTGAAATGG |
| rs1064190 | 31130758 | ACGTTGGATGAATGCAGTGCGTTGTCCCAG | ACGTTGGATGAACTACAGCCTCTGCACCAG |
| rs3132549 | 31142686 | ACGTTGGATGTTTCACCATCTTGGCCAGGC | ACGTTGGATGCTTGTGCCTGTAATCCCAAC |
| rs1265103 | 31156625 | ACGTTGGATGGGCACAAAAATGGTAAAGGG | ACGTTGGATGTCATGTCTGTCTTCCCTTCC |
| rs1265099 | 31161025 | ACGTTGGATGTCTACTGATAGTTCCTGCCG | ACGTTGGATGTAAGCCTACTCTCCTACCTC |
| rs1265114 | 31173035 | ACGTTGGATGATCCTACCTGAGGCTGACTC | ACGTTGGATGCTGGGTGACAAAGCGAGATC |
| rs1265112 | 31173867 | ACGTTGGATGTCTGAAGGTTGAACCTGAGG | ACGTTGGATGACAAAGATGCCACCTCCTTC |
| rs130078 | 31174413 | ACGTTGGATGAGTTCCCATGTCTGGCTGTG | ACGTTGGATGGGTGACCCTGGTTGAGAATC |
| rs2240059 | 31176474 | ACGTTGGATGGTTCTGAAGTGGCCAAAGCC | ACGTTGGATGGCACTGAGTGTGCTGCAGAG |
| rs130075 | 31178112 | ACGTTGGATGTGATCGTTCGGCAGCTGCAAG | ACGTTGGATGTCATCTTCTGCTGCAGCGAG |
| rs130076 | 31178340 | ACGTTGGATGATCGTTCGGCAGCTGCAAGAG | ACGTTGGATGTCATCTTCTGCTGCAGCGAG |
| rs130065 | 31178358 | ACGTTGGATGATCGTTCGGCAGCTGCAAGAG | ACGTTGGATGTCATCTTCTGCTGCAGCGAG |
| rs2073716 | 31178855 | ACGTTGGATGAGGTTGGAAGAACACACAGG | ACGTTGGATGCCATTCCTCCCTCAAACTTC |
| rs720466 | 31181582 | ACGTTGGATGTGAAGCCTCGGGTATCTAGG | ACGTTGGATGATTCTGGTCCTGACCCTCAC |
| rs720465 | 31181654 | ACGTTGGATGTCTCTCAATAGCCTGCCCTC | ACGTTGGATGTAGAGCTCACGGGCTAACTG |
| rs1265162 | 31193347 | ACGTTGGATGCCCAAACAGGAGATCCTATC | ACGTTGGATGCCTGAGGGTAAAAACAGTGC |
| rs915660 | 31199448 | ACGTTGGATGGTCTTGGAGAATGAGTGAGG | ACGTTGGATGTCCTACCTCCTCCCAAAATG |
| rs885701 | 31199563 | ACGTTGGATGTCTTCTCTGTCAACCACATC | ACGTTGGATGAGTGCATGCTGGGTACATGG |
| rs1052989 | 31202267 | ACGTTGGATGGGAGGCACTAAATATTCACG | ACGTTGGATGTTGAAACCTCCTGCATCCTG |
| rs1265181 | 31211656 | ACGTTGGATGTTTGGCCTAGTTTGAGTGCC | ACGTTGGATGGCTGCACAAACAACTTTCGC |
| rs886389 | 31222612 | ACGTTGGATGAGAAAGAAAGAAGAGAGAGAG | ACGTTGGATGGTCCATTGAATGGAGTATAGC |
| rs1793899 | 31225739 | ACGTTGGATGACCTCTCTGCTCTCTGTCTC | ACGTTGGATGTCCTTGTCAGGGACCACAAG |
| rs3132502 | 31239363 | ACGTTGGATGCAAGACTCCTTTCCTGTAAC | ACGTTGGATGATCGTGCCATTGCACTCTAG |
| rs1793895 | 31247789 | ACGTTGGATGTCTGAACCCACACAGTACAC | ACGTTGGATGTGGCACAGTCAGAATAAGGC |
| rs1793894 | 31252511 | ACGTTGGATGTTTCTCCATGTTGGTCAGGC | ACGTTGGATGAATCTCAGCACCTTGAGAGG |
| rs3134756 | 31269208 | ACGTTGGATGAAAACATTGCAGGAGCTGAC | ACGTTGGATGCAGCTTTATCAGGTTGGTTTC |
| rs1793893 | 31272501 | ACGTTGGATGTACCATGAATATAGCTATCG | ACGTTGGATGTTTGCCTGAAGGACTGAAAC |
| rs2394948 | 31275364 | ACGTTGGATGGGGTCTAGAGAAGTAGGTTG | ACGTTGGATGGGCAATACAGCTGCATTCAG |
| rs1793892 | 31275440 | ACGTTGGATGTTTGCATCCCTAGTCCTGAG | ACGTTGGATGTACAATCCTTCCCAAGGTGG |
| rs3130542 | 31286439 | ACGTTGGATGGTCTGCTAAACACAGGTTTC | ACGTTGGATGTTATGTGACCCCCTCAAAGG |
| rs2040748 | 31297875 | ACGTTGGATGAGCAATCACAGCAAAGGAAC | ACGTTGGATGTCAGGAACACTGAGAGAATG |
| rs2253288 | 31301099 | ACGTTGGATGCAAAGCCACAATGAGATACC | ACGTTGGATGAGCCTCACCAGCATCTATTG |
| rs2253487 | 31303455 | ACGTTGGATGTCATGCTGAAAGGCTGTGTG | ACGTTGGATGAGGTCAATCTTCTCCAGAGC |
| rs2853941 | 31303557 | ACGTTGGATGGTGGTCCCATGAATGCTTTC | ACGTTGGATGAAGTTCATTGACACCCCCTC |
| rs2844604 | 31304836 | ACGTTGGATGCTGAAAGTGGACTGTGAAATG | ACGTTGGATGTGAGACTCAAGACTGGCTAG |
| rs2853939 | 31304971 | ACGTTGGATGAAACCCTAGCCAGTCTTGAG | ACGTTGGATGTAACTCCTCTTTCTGGGCAG |
| rs2524059 | 31305152 | ACGTTGGATGCAGTGACTTTGTTGCCTTGC | ACGTTGGATGTTCTCCAAGTGTGGACACAG |
| rs2844603 | 31305183 | ACGTTGGATGATTCCACTTTACCCAGTGTC | ACGTTGGATGTCAAGGTTTCTTTCTCCAAG |
| rs2853938 | 31305806 | ACGTTGGATGCCTGGAGGATGAGCAATGAC | ACGTTGGATGTTGCAGTGCTCCTGCTCCCA |
| rs2524058 | 31305898 | ACGTTGGATGTGGGAGCAGGAGCACTGCAA | ACGTTGGATGAGAAATCCCAAGGAGAGGCC |
| rs2524053 | 31306798 | ACGTTGGATGGACTTTTACGATCATCACTTC | ACGTTGGATGTTTCAAGGAAGAATCTATAG |
| rs2853935 | 31308207 | ACGTTGGATGCTATAATCAAAGCCTGGGAC | ACGTTGGATGGGAAATGCAAGAATGAGAGC |
| rs2853933 | 31308417 | ACGTTGGATGTTCCCTCATGTTGTTGCTGG | ACGTTGGATGACAGCTACGGGTCTATCAAG |
| rs2524151 | 31316283 | ACGTTGGATGCCTTCAGATAAGGTATTGGG | ACGTTGGATGTTGGATCAGCAGCTCTTTTG |
| rs2524123 | 31319639 | ACGTTGGATGTCCCCAAGAGGTTTTCACAG | ACGTTGGATGCTGCAGTGGTAGAAGAGAAG |
| rs2247056 | 31319815 | ACGTTGGATGTGCATGGCTGTAAATTAGGC | ACGTTGGATGAGGGCTGTCTAATCATTCCC |
| rs2524089 | 31320847 | ACGTTGGATGCCCCTTCCTTGTATAGTTCC | ACGTTGGATGTACAGGTCTGTCCCACCATC |
| rs364415 | 31327348 | ACGTTGGATGTTGAACCATGAGGAGGAGTC | ACGTTGGATGTCTCCTCTCACACCATCCAG |
| rs3130690 | 31340260 | ACGTTGGATGATGAGGTCATGTGAGTGTGC | ACGTTGGATGTTCCTCCGTATCTGTCTGTG |
| rs2524227 | 31356125 | ACGTTGGATGAAAGAGAATGCCCTGAATGG | ACGTTGGATGAAAAAGAGTAGAGCCCCTGG |
| rs2854008 | 31366306 | ACGTTGGATGAAGACCCATTTGCTGCTTCC | ACGTTGGATGTGGGAGGGCCTTGAAAATAC |
| rs709052 | 31376822 | ACGTTGGATGAGATCACACTGACCTGGCAG | ACGTTGGATGTTCTATCTCCTGCTGGTCTG |
| rs2250295 | 31384198 | ACGTTGGATGGAAAACAAATCCTAGCCAGTC | ACGTTGGATGCGATAGTTCTGAAATCGTAGG |
| rs2596548 | 31384333 | ACGTTGGATGAAATATGGTGTCCCTGGGAC | ACGTTGGATGGAGTGGAAGAGCAAGACAAC |
| rs2853996 | 31386696 | ACGTTGGATGCCATCATCCCTCACTTGAAC | ACGTTGGATGGCCACCCCAGATCTTTATTC |
| rs2596438 | 31393338 | ACGTTGGATGAAGTATGACTCATTCACAGG | ACGTTGGATGGTCCATTGTTCTTCAGGAAC |
| rs2853976 | 31399027 | ACGTTGGATGTACTTCTGATCCCCTAGGAC | ACGTTGGATGAGCAGCCTTCCATAGACATC |
| rs2244020 | 31401021 | ACGTTGGATGATGAACAGGACCTTCCACCC | ACGTTGGATGAGCCACCACACCTTCTTCTG |
| rs2523466 | 31416809 | ACGTTGGATGTATAACTGTCCCAGCTCCTG | ACGTTGGATGTAGGAAACATCCCCACCTAG |
| rs2523454 | 31421660 | ACGTTGGATGTACTCACCCGGATCAGAATC | ACGTTGGATGATGAAAATGCAGACCCGCAG |
| rs2301749 | 31425152 | ACGTTGGATGTTCATTGGATGAGCGGTCGG | ACGTTGGATGTCTCAGCGGCTCAAGCAGTG |
| rs2301747 | 31425383 | ACGTTGGATGTGAAGTGTGGCGGTAACGGG | ACGTTGGATGTGCTGGTGAGTGGCGTTCCT |
| rs2256184 | 31434180 | ACGTTGGATGTCTCTTGAACTCACTAGGGC | ACGTTGGATGACTATTTGCTCCCTCTGAGG |
| rs2848716 | 31441607 | ACGTTGGATGTGAAACCCCAATGTCTCACC | ACGTTGGATGTGAGCCCAGAGTTGACAGAG |
| rs2516446 | 31446421 | ACGTTGGATGTCAAGTGATCCTGCTCTCTC | ACGTTGGATGTAGTAAAGAGGGCAGGCATG |
| rs2516470 | 31460915 | ACGTTGGATGAGTTAAGAGATTCCCTGACC | ACGTTGGATGAAAGACAGCACATTCTGCCG |
| rs3099847 | 31476996 | ACGTTGGATGAGGGGCTCCTCACTTCCCAG | ACGTTGGATGTCAGCTCCCCGCCCAGCCA |
| rs2596552 | 31477071 | ACGTTGGATGAGGCAGAGGGGCTCCTCAC | ACGTTGGATGTGAGGAGCGTCTCCGCCCG |
| rs2596472 | 31482726 | ACGTTGGATGTTTACCAGATGTCTGAAAGG | ACGTTGGATGTATCAATTCGCCCATTGCAG |
| rs2523674 | 31490746 | ACGTTGGATGCAGAAAAGACTGGGAAAGCC | ACGTTGGATGTTGCAGTGAGCTGAGATTGC |
| rs2904786 | 31510355 | ACGTTGGATGGTTGATGGCACCTTCAGAAG | ACGTTGGATGAAACCCAAAGATGGGTCAGG |
| rs2855804 | 31521290 | ACGTTGGATGTTCTGGTGCTGCCTTTTGTC | ACGTTGGATGAACTGCCATTAGCATCAGGG |
| rs3132464 | 31531399 | ACGTTGGATGTTTCTCTCTTCAGTTGCCCC | ACGTTGGATGGGGAGGAAGAAAAAAGTGGG |
| rs2516400 | 31553393 | ACGTTGGATGAGGTGGACAAATCACAGGTG | ACGTTGGATGTCAACGGTGTTTCTTGGAGG |
| rs3130638 | 31560032 | ACGTTGGATGTGAGGTCAGGAGTTCAAGAC | ACGTTGGATGCCATGCCTGGCTAATATTTG |
| rs11796 | 31564553 | ACGTTGGATGTTTTGACTGTCCATTGCAGC | ACGTTGGATGCGTGTGCATTAGCAAAGTGG |
| rs2239709 | 31570511 | ACGTTGGATGTAGAGATGACTGGCTTCTGG | ACGTTGGATGTTGCTATACTTCGGGTCACG |
| rs2857607 | 31580348 | ACGTTGGATGACTTTGAGAGGCTGAGGTTG | ACGTTGGATGTTTCGCCATGTTGGACAAGC |
| rs2230365 | 31588298 | ACGTTGGATGTACACCGATTTCTTCCTCCC | ACGTTGGATGGGGTCTCCCCATCCTTATTC |
| rs2844490 | 31595707 | ACGTTGGATGGCCTTTTGCATTTGCCATGC | ACGTTGGATGGTGGAGAAAGACTGAGCTAG |
| rs736160 | 31601908 | | |
| rs361525 | 31606263 | ACGTTGGATGATCAAGGATACCCCTCACAC | ACGTTGGATGACACAAATCAGTCAGTGGCC |
| rs986475 | 31619673 | ACGTTGGATGGTCCCTGAACACTGTCATTC | ACGTTGGATGAAACACATGGCTCACCCTTC |
| rs2857595 | 31631860 | ACGTTGGATGTTGATAAGACTTGGCCAGAG | ACGTTGGATGTGATCTCATCTTTCCCCCAC |
| rs2051552 | 31643098 | ACGTTGGATGGCCAACATAGTAAAACCCCG | ACGTTGGATGAAGTGATTCTCCTGCCTCAG |
| rs2844476 | 31645038 | ACGTTGGATGATTGCACCATTGCACTCCCG | ACGTTGGATGAGATGCTGGAGTGGCCTCTG |
| rs2736181 | 31646906 | ACGTTGGATGTCTCTCAGCATCCCCTCTAG | ACGTTGGATGAGACAACGTGGAAGGAGGAG |
| rs2736160 | 31662291 | ACGTTGGATGATAACTGGCCAGATAGGGTG | ACGTTGGATGCTTTTCCCACCTAGTTCTGG |
| rs750332 | 31670185 | ACGTTGGATGTAAGCAGGTTGGAGAAACGC | ACGTTGGATGTGTTAGCTTCTGAGGGATGG |
| rs1052486 | 31673871 | ACGTTGGATGACAGTGATGGTGGGAGAAGC | ACGTTGGATGTCAGTTCTCTCAGCTTCTGG |
| rs3117583 | 31682962 | ACGTTGGATGCCGACAGGTCTCTAAAGAAG | ACGTTGGATGAGTCTTTCGGGTACACTCTG |
| rs2894225 | 31692409 | | |
| rs928814 | 31695049 | ACGTTGGATGGGTTCCAGCAGTCTCCTAAG | ACGTTGGATGAGATGACTCACCGGATACTG |
| rs805256 | 31698902 | ACGTTGGATGTCCAGGTCCAAGATCATGTC | ACGTTGGATGTACTGGACTCAATGAGCAGG |
| rs805290 | 31711788 | ACGTTGGATGGAGACTTTGTGCAGGGTTGT | ACGTTGGATGGGGAATGAGAAAAGGAACTG |
| rs805281 | 31724677 | ACGTTGGATGCCTCTTCCAAGCTAAGAACC | ACGTTGGATGAAAGCACTAGCACCTTCAGC |
| rs805289 | 31740426 | ACGTTGGATGAGTAGCTGGACTACAGGTGC | ACGTTGGATGACAGAGAGAGACTCTGTCTC |
| rs376510 | 31751388 | ACGTTGGATGGTGGAGTGACGGAAGATATG | ACGTTGGATGAGGTAAGGGTAGAGCTGTTG |
| rs805292 | 31753147 | ACGTTGGATGTTAATCTCCATTCAGCCCCC | ACGTTGGATGAGAAGCCATCAGTGAGTCAC |
| rs3131382 | 31771118 | ACGTTGGATGTCGGATCTCTAGGCTGGATC | ACGTTGGATGACGAGCCTGCAAAAGGAGCG |
| rs1150793 | 31789133 | ACGTTGGATGAATCCTTCCCCTACCTCACC | ACGTTGGATGTTACCTGGAGATGACCTCAG |
| rs707935 | 31806157 | ACGTTGGATGTACATTTATTCCCTGAGCCC | ACGTTGGATGGTTATGCATATGCACAGATG |
| rs707932 | 31810029 | ACGTTGGATGAGGAGAATCACTTGAACCCG | ACGTTGGATGCCTGCACTGACAAGTATGAC |
| rs707928 | 31814030 | ACGTTGGATGCCTGTGCTGTGTTTTCCAGC | ACGTTGGATGAAAACCTAGGATCATGGGCC |
| rs1150749 | 31830029 | ACGTTGGATGAATCGCTTGAACCTAGGAGG | ACGTTGGATGACTCTTTTTGCTCAGGCTGG |
| rs480092 | 31836340 | ACGTTGGATGTACCATACCTGCAACTGGAG | ACGTTGGATGTGGTGAAGTCTGGTAGCATG |
| rs2075799 | 31850226 | ACGTTGGATGTTATCAGGGCAGTCATCACG | ACGTTGGATGAGTCTGAGAAGGTACAGGAC |
| rs539689 | 31857089 | ACGTTGGATGATCCACCTCCTCAATGGTAG | ACGTTGGATGTGTGTAACCCCATCATCAGC |
| rs2763979 | 31866094 | ACGTTGGATGATCTTACTCGGGACTGTGAG | ACGTTGGATGCACCTCCTTCCTACTTTCTC |
| rs3130679 | 31879245 | ACGTTGGATGCATTGTTCTGAGACCAAACC | ACGTTGGATGGGGCACTTCAAGTAGATAGC |
| rs574914 | 31890829 | ACGTTGGATGGCCAAGATGGAAGTTAAGCC | ACGTTGGATGGGAGAGTCTGTAAGAAGCAG |
| rs2021007 | 31890874 | ACGTTGGATGGGAAGTTAAGCCTTGGAGAC | ACGTTGGATGGTGATTGGAAAGGAGGTCTC |
| rs660550 | 31909117 | ACGTTGGATGGTGACACCAAGGCACTCTAC | ACGTTGGATGACTGCTCTGTATCCTCTGCC |
| rs605203 | 31918651 | ACGTTGGATGTTTAATCTTTGGCGGGAGCG | ACGTTGGATGACCTGGCATGCTCTGATAAG |
| rs589428 | 31919809 | ACGTTGGATGATGGGATCTGAGCCCCTTGT | ACGTTGGATGTGTCCAGGCATGGAGTAGTG |
| rs612496 | 31931785 | ACGTTGGATGCAATATCACGATCTGGGCTC | ACGTTGGATGAGGCTGAGGCAGGAGAATTG |
| rs558702 | 31941915 | ACGTTGGATGTGTGAGCACACTCAGCAGAG | ACGTTGGATGTGCATCCGTGGCACCTCTCA |
| rs2763982 | 31944190 | ACGTTGGATGTCCAGTGAGAGCAGAAATAC | ACGTTGGATGTGTCCCATCTACATTCCTAG |
| rs3020644 | 31956403 | ACGTTGGATGCACTTCAGAGAGGTTTCATG | ACGTTGGATGTGACCCACAGAAGTCTTTTC |
| rs1265911 | 31962348 | ACGTTGGATGAGTGCTCAATGATGCCCAGG | ACGTTGGATGAATCTCGGCACTTTGGGAGG |
| rs2854340 | 31963783 | ACGTTGGATGTGTCCCAACAGTGCTTGTGG | ACGTTGGATGCCTCCAAGAAGTCTTCTCAG |
| rs609061 | 31971951 | ACGTTGGATGCCTGTTTATTCCCTGTAATGG | ACGTTGGATGTGATTACAGGTGTGAGCCAC |
| rs1270942 | 31980650 | ACGTTGGATGTAGCTCTAGAAGGGCTTAGG | ACGTTGGATGATAGACTGCGTCACTTCAGC |
| rs419788 | 31990590 | ACGTTGGATGCCTTTCTTGAAACCAGGTGG | ACGTTGGATGTTGTTCACCAGTGTGCAGTG |
| rs429608 | 31992054 | ACGTTGGATGACTGTACAGCATGGAGCTGG | ACGTTGGATGAGAGTGTGCTGTCTGAGGAG |
| rs416002 | 32000424 | ACGTTGGATGACATGTGTGCAGGTGAGTTG | ACGTTGGATGTGTCTGCACAAGGAGAGAAG |
| rs2746392 | 32009805 | ACGTTGGATGTCTGTTGGTGCAGTTGCTTG | ACGTTGGATGGGAAAAGAAAAAGTGGAGGG |
| rs2734323 | 32020483 | ACGTTGGATGAGCTCACTGTCTTGTGGGAG | ACGTTGGATGCAAGAAGAGAGGGACAGGAG |
| rs3130677 | 32034947 | | |
| rs433061 | 32043622 | ACGTTGGATGTTCCCAAACCCTCACTGTGG | ACGTTGGATGGGGAGAAGACAGGGGATTAG |
| rs916139 | 32046588 | ACGTTGGATGTCACCATCTCAGGCCTGGAG | ACGTTGGATGCGTGGAAGCCGTACAGGTTC |
| rs3117189 | 32078519 | ACGTTGGATGCCCAGGCTGATATCAAACTC | ACGTTGGATGATCCCAGCACTTTGTAAGGC |
| rs204879 | 32087748 | ACGTTGGATGAGAGCAAATGCAGAGACTGG | ACGTTGGATGCTCCAACTCCACCACAAAAC |
| rs2021783 | 32089404 | ACGTTGGATGGAAGGAACTCAGTTTGTCAG | ACGTTGGATGTGTCAGGCACTGACCAAGTC |
| rs2239688 | 32098814 | ACGTTGGATGAAGGCTTCCATGACCTCCAG | ACGTTGGATGAATGGAAGCCACCTGACCAC |
| rs204896 | 32108695 | ACGTTGGATGACAGTCCTCACCGGTGAAGC | ACGTTGGATGATGTGTTGGCCGGGGTACAC |
| rs393544 | 32118867 | ACGTTGGATGTCTCTTACCCAAGGCTACAC | ACGTTGGATGATACAGAGAGCTGCCCTTTC |
| rs1269852 | 32119789 | ACGTTGGATGTCCAAACCCTTCCTTCTGAG | ACGTTGGATGCAGGATGAAAGATGGGAGAG |
| rs204894 | 32134359 | ACGTTGGATGGGAGATGACCCAACATCCTC | ACGTTGGATGCTGAGGAATCATCAGAGGTG |
| rs421602 | 32135422 | ACGTTGGATGTCTCCTTTACAGCTTGGTGC | ACGTTGGATGGTAACAGAGGCAGCTGTTTG |
| rs2071291 | 32140276 | ACGTTGGATGGCACGTAAGCAGTGCAAGGC | ACGTTGGATGTTCGTGGTGCCCACGCACG |
| rs2269425 | 32150480 | ACGTTGGATGGGGAACAGAGGTTTATGGTC | ACGTTGGATGACAGCCACTTCAAGTAGTCC |
| rs1269839 | 32163718 | ACGTTGGATGAAACCCTCTTCCTTGTCTCC | ACGTTGGATGGCTGTATCCACATTCACTTC |
| rs408359 | 32168920 | ACGTTGGATGCACTGATCTTGACAACACAC | ACGTTGGATGAAGCTGCTTACAGCCTAAGG |
| rs204996 | 32176422 | ACGTTGGATGTCTGGCCTTATCCCTAACAG | ACGTTGGATGGCTCTCTTGGCAGAATTTGG |
| rs204989 | 32188886 | ACGTTGGATGGAAACATGGAGTCATGAGGC | ACGTTGGATGGCACCTACTTCATAGGGTTG |
| rs2071280 | 32191654 | ACGTTGGATGACTGCAGTTTGTCTGCTACG | ACGTTGGATGTAAGGAACTCGAGTTGGCAG |
| rs2071277 | 32210496 | ACGTTGGATGAGGAATGAGCTAGGATGGAG | ACGTTGGATGCACTGGCCTGTAATTATGGG |
| rs2856433 | 32221133 | ACGTTGGATGCTCTCGTAGAGCTTTCATTC | ACGTTGGATGACCTGCTCATTTTCTCCAAC |
| rs375244 | 32230293 | ACGTTGGATGAAATAGAGACGGCCTCCAGG | ACGTTGGATGTGACGAGGTTCTTCCTGGAG |
| rs2849015 | 32237569 | ACGTTGGATGAAACTGCTCATCACCACACC | ACGTTGGATGATGCCCCAATCATCTCTCAC |
| rs3130316 | 32250273 | ACGTTGGATGCTTCCTTTCCAATCTTTTGG | ACGTTGGATGCAAGGAGACTACTATCATCAC |
| rs1150763 | 32253575 | ACGTTGGATGTGGGACTATGTGAAAAGACC | ACGTTGGATGTTGATTCCATTCTCCCCGTC |
| rs3130338 | 32270791 | ACGTTGGATGAGAGAAGCAGGAGTAAGGTG | ACGTTGGATGCAGCATCACTAAGGATCAGG |
| rs1265788 | 32281025 | ACGTTGGATGAGTCAGGAAACAACAGATGC | ACGTTGGATGTTACACTCCCATCAACAGTG |
| rs926070 | 32283275 | ACGTTGGATGGCCTTTAGAAAATGGTCCAC | ACGTTGGATGTCCCACTAGGTCTTTGAAGG |
| rs2038191 | 32290024 | | |
| rs491870 | 32298352 | ACGTTGGATGAAAGAATTGGGACTTGCCTC | ACGTTGGATGTGGTGTATTTAGACCTACAC |
| rs1018433 | 32308410 | ACGTTGGATGTGTCCTAACTTCCTGGGTAC | ACGTTGGATGTGTGCTACCCATGCAGTGTG |
| rs513095 | 32314754 | ACGTTGGATGATCACTCACCACTCACATGG | ACGTTGGATGGCCCCCAAGGAATAAGAAAC |
| rs742697 | 32318423 | ACGTTGGATGAGGGAAAACTTTCCCTTTGG | ACGTTGGATGGGGTGCATACTACTTTAACC |
| rs523627 | 32318719 | ACGTTGGATGTGACCTGCTGATAAACTTTC | ACGTTGGATGTGACACATCATTCTCTCACC |
| rs2077333 | 32320455 | ACGTTGGATGACCACCACCTAAGTTTCCAG | ACGTTGGATGAAGCAAGAGATGGCTAGTGC |
| rs2395143 | 32320463 | ACGTTGGATGACCACCAAAGTTTCCAGAAC | ACGTTGGATGCTGTCAAAGCAAGAGATGGC |
| rs504703 | 32320902 | ACGTTGGATGAGAAGTGACAGGGAAGCTAC | ACGTTGGATGTTCTTTGTACCAGCTAGGCC |
| rs3129958 | 32327056 | ACGTTGGATGACTGATGGTAGGGAAAGGTG | ACGTTGGATGAGAACAGTCCCTTGAGAAAG |
| rs3129960 | 32327712 | ACGTTGGATGATCATGCCACTGCATTCCAG | ACGTTGGATGTTTCTAGCTCTGATCGCCTG |
| rs2022537 | 32328814 | ACGTTGGATGCTTGGATAGGTGATCACTTC | ACGTTGGATGAGGGAAATGAGTATGTTGAG |
| rs2022534 | 32333790 | ACGTTGGATGCTCTCTTTTACCAGTGTGAG | ACGTTGGATGCAGTCACTTAGAGGATCTTG |
| rs2143468 | 32335906 | ACGTTGGATGTATCCACAGAGACAATGTCC | ACGTTGGATGGGGCAGTGGAAGGTATTTAC |
| rs2395145 | 32338774 | ACGTTGGATGCTCACCATCTTTTGGAACTG | ACGTTGGATGAAACCCTGTCATTGATCGAC |
| rs2076542 | 32343684 | ACGTTGGATGTACATGGCTAGCACGAAAGG | ACGTTGGATGATCTCTTCCATTGCTGCCAG |
| rs2076541 | 32343772 | ACGTTGGATGGGATAAGAGCAAAAAGTTAG | ACGTTGGATGCTGAGGACACAGCTAATATC |
| rs2076540 | 32343828 | ACGTTGGATGGAGAGCAATTTCCAAACCTG | ACGTTGGATGCTAACTTTTTGCTCTTATCC |
| rs3129907 | 32350292 | ACGTTGGATGGTTATAAGGTAAGTTGAGGTC | ACGTTGGATGTGAATTCTCAGTCAGCTGAG |
| rs3129927 | 32360382 | ACGTTGGATGCCTGCCACAACATAAAAGGC | ACGTTGGATGAAATGGTGCCTCATAGCGTG |
| rs2143462 | 32361583 | ACGTTGGATGTTAGTGGTACTGGTGTGTCC | ACGTTGGATGCAGGTTTTGAAACGTGAGAG |
| rs2073047 | 32362481 | ACGTTGGATGTTGGTGATTGACACAGTCAC | ACGTTGGATGGCAGGAACTAGGAATTGTGC |
| rs2073044 | 32365548 | ACGTTGGATGGTACTGAGTACACCATCTAG | ACGTTGGATGCAAGTAGTCAATATGCCCTC |
| rs2050190 | 32365638 | ACGTTGGATGCCCTATTAATAGGGTGGACC | ACGTTGGATGAGTGTCTGAAATGCCCTGTC |
| rs2076536 | 32365910 | ACGTTGGATGTCCTTGCCTGCTTCCTTTTC | ACGTTGGATGTAACTGTGGGTTGTTTCCCC |
| rs2050189 | 32366209 | ACGTTGGATGGCTTAGGTCTGATCAATCTG | ACGTTGGATGTATGAACTTGGGTGTCAGGG |
| rs2395151 | 32369884 | ACGTTGGATGAAAACGATGCCCCTATCAGC | ACGTTGGATGGTACGTCTAACTGCTGTTCG |
| rs2894252 | 32371988 | ACGTTGGATGGGGAAAGAAAATGTCTATGGC | ACGTTGGATGTAGATGAGAGTGCAACTTCG |
| rs2395156 | 32374635 | ACGTTGGATGTTGCACAGATGCAAAGATTC | ACGTTGGATGAAATGTTTGTGCCATCTAAG |
| rs2395157 | 32374682 | ACGTTGGATGTTTAAAATGTTTGTGCCATC | ACGTTGGATGTTGCACAGATGCAAAGATTC |
| rs1555115 | 32381050 | ACGTTGGATGATCTATTCCAGCCAGGCTAG | ACGTTGGATGCCCATCCTGAAAACCTTACC |
| rs2076534 | 32388689 | ACGTTGGATGTTTGCAGAGGATAGCAGGAG | ACGTTGGATGAGACCAACTCAGACTTACTC |
| rs2076533 | 32390050 | ACGTTGGATGCTAATAACACACTGTGAAAC | ACGTTGGATGAGGAAATCTGAGTATCTTAC |
| rs2076530 | 32390339 | ACGTTGGATGAGGCCAGTTTGGATCTGAAG | ACGTTGGATGATTAAAGTGGCAGGAGCAGG |
| rs2076529 | 32390478 | ACGTTGGATGTCAGTCTGCCCTCGTCAATG | ACGTTGGATGGAGAGCAGATGGCAGAGTAC |
| rs2294880 | 32394245 | ACGTTGGATGACCTGACAGGAAGCAAAGGG | ACGTTGGATGTAAGTCATGGTAACCTCCGG |
| rs2294878 | 32394318 | ACGTTGGATGTAGGAACAACAGGACATGGG | ACGTTGGATGTCCTCTGAGTTCTCTGAGAC |
| rs2076525 | 32397124 | ACGTTGGATGGCACCTCGTATTTTTATCAAG | ACGTTGGATGTGGCTTTCAATACATATTGC |
| rs2076524 | 32397192 | ACGTTGGATGTACGAGGTGCTATGGTGCAG | ACGTTGGATGAGGTCAGTGCTCTGCCTCTAG |
| rs2076523 | 32397343 | ACGTTGGATGTATTGGGAAGACATCCGGG | ACGTTGGATGTGGCTTCCGCATAGAACAGG |
| rs2395158 | 32401103 | ACGTTGGATGGCTGAGTCACCTTTGGAAAG | ACGTTGGATGGGCCTCTGAGATGTAGTTAC |
| rs3135380 | 32411189 | ACGTTGGATGTAAAATTGGGCATGGGAAAC | ACGTTGGATGGAAATCTGCTAGGCTTAAAC |
| rs2395161 | 32414066 | ACGTTGGATGTTTCCCTCCCCACAATCTAC | ACGTTGGATGTCACCTGGACCTGATTGATC |
| rs2395163 | 32414323 | ACGTTGGATGATCGGCAGCTTGGAAACTAC | ACGTTGGATGGGGCTGGATAATGATGGATG |
| rs2395165 | 32414658 | ACGTTGGATGCAGCTTCCATGTGGTGTTTG | ACGTTGGATGTTTGTCCCTCTAGCCCTTTG |
| rs2395166 | 32414789 | ACGTTGGATGCAGTTCCTATGAAGGATGATC | ACGTTGGATGCCATAGAAACCTTGGAAGTC |
| rs2213581 | 32415060 | ACGTTGGATGCAGTATCCCACAGAGAAGTC | ACGTTGGATGGGAGCCTCAAATTATCACTC |
| rs732163 | 32421456 | ACGTTGGATGACCCCTTTCTAATATCTCTC | ACGTTGGATGTCTTCTATATCGGATAATGC |
| rs732162 | 32421458 | ACGTTGGATGACCCCTTTCTAATATCTCTC | ACGTTGGATGTCTTCTATATCGGATAATGC |
| rs1894552 | 32422010 | ACGTTGGATGGCTCTTCAACTTATGATGGG | ACGTTGGATGGCCACATGATCATGAAGGTG |
| rs2105903 | 32422201 | ACGTTGGATGAAACTACAGACACACCTGAC | ACGTTGGATGTCACCTTCATGATCATGTGG |
| rs983561 | 32430210 | ACGTTGGATGTCATATTGGCCACTCCGAAG | ACGTTGGATGTGAGAAGATGAGAGCAACAG |
| rs3129868 | 32430931 | ACGTTGGATGTATTCCAGCAGACCAGCTTC | ACGTTGGATGGAGGTGCTGAGGGAATATTG |
| rs2395173 | 32431414 | ACGTTGGATGTACATCTCTCAGGCTTGCTC | ACGTTGGATGACTTCCACCTCCCAAATCTC |
| rs2395174 | 32431433 | ACGTTGGATGTACATCTCTCAGGCTTGCTC | ACGTTGGATGACTTCCACCTCCCAAATCTC |
| rs2395177 | 32431631 | ACGTTGGATGATCTGCAACATCAGCAGAGG | ACGTTGGATGAGCCCTTAAAACTGTTAGGG |
| rs2239804 | 32438079 | ACGTTGGATGTGTTACTTCTTCCCACACTC | ACGTTGGATGGCTTGGAGCATCAAACTCTG |
| rs2239802 | 32438402 | ACGTTGGATGCTGAAGCTTTGGGATACCAG | ACGTTGGATGAGGAACAGATGTGGCTCTTG |
| rs1051336 | 32438914 | ACGTTGGATGAGTGTGGATATGCCTCTTCG | ACGTTGGATGGGAAAAGGCAATAGACAGGG |
| rs3177928 | 32438957 | ACGTTGGATGGGTAACTATGTGTGTCTTGC | ACGTTGGATGGCAGAAGTTTCTTCAGTGATC |
| rs7194 | 32439002 | ACGTTGGATGCATGGAGGTGATGGTGTTTC | ACGTTGGATGTGCTTTCACTGAGGTCAAGG |
| rs2213586 | 32439616 | ACGTTGGATGTCTGAGATCCATACCTTGGG | ACGTTGGATGTTGGGAGATCTCTACTGAGC |
| rs2213585 | 32439672 | ACGTTGGATGAACCCCAAGGTATGGATCTC | ACGTTGGATGTTCCTTCTCCCCACTCTAAC |
| rs2213584 | 32439781 | ACGTTGGATGAATGGGTTAGGCCAGTCTTC | ACGTTGGATGGAAGGAAGACAGAAGAATCC |
| rs2395182 | 32439839 | ACGTTGGATGGGCCTTACCCATTCTGTTAG | ACGTTGGATGTCAGTCAGACTACTCTCTCG |
| rs2227139 | 32439981 | ACGTTGGATGGACATTAAGATGAGAGGAAGG | ACGTTGGATGTGGTTTATGGCAGGTTCTAG |
| rs1547422 | 32453362 | ACGTTGGATGTGCATAAGCATTTCACTGAG | ACGTTGGATGCAAACCTGTACATGTATCCC |
| rs1548306 | 32453442 | ACGTTGGATGATAATGTGAGGAGGCTAGTC | ACGTTGGATGATTTCAGAGATTTCGGGATC |
| rs2187824 | 32465527 | ACGTTGGATGCTCTAGCCTTCTTTCTGTCC | ACGTTGGATGTTCCAGGGAGACAGAATGTG |
| rs2187823 | 32465789 | ACGTTGGATGCCAGGATCCAAACAGTGATC | ACGTTGGATGAGTACACAGTAGCTGCTGAG |
| rs2187822 | 32475997 | ACGTTGGATGACCAGGCCTTTGATTTTCAG | ACGTTGGATGACTACATTTGGGATACTGGG |
| rs1974460 | 32480175 | ACGTTGGATGAGCAGGCAAGTCTCACATTC | ACGTTGGATGGTACCTTACTCCCTGTGTTG |
| rs2395199 | 32482024 | ACGTTGGATGTCAGTGCAGTCAGCTGCCTC | ACGTTGGATGAGCCACTGAGGGAGTAGTGG |
| rs2894266 | 32491452 | ACGTTGGATGGCAAATCTGTCCTCCAACAC | ACGTTGGATGGGTGTGGGTTTTGGTGTTAG |
| rs2213583 | 32499701 | ACGTTGGATGTCTGTCTCAGCCCACTTTGC | ACGTTGGATGGTGGAAGAGGATACATAGGG |
| rs1987529 | 32502240 | ACGTTGGATGCCAGTTTTTCAGAGGATGCC | ACGTTGGATGCTGGAACTGAAGCTGAGATC |
| rs2395210 | 32502763 | ACGTTGGATGTTCCCCATACAGCAATTCCC | ACGTTGGATGATAACCCAGGATCGTCTAGG |
| rs2071807 | 32503145 | ACGTTGGATGTATATTCCCCCACCCCATAG | ACGTTGGATGCGTTGACAGTGACACTGATG |
| rs2071806 | 32503501 | ACGTTGGATGGCAACTGGTTCAAACCTTTC | ACGTTGGATGGCTGTATGAAGGTCCTCTTC |
| rs2187821 | 32504679 | ACGTTGGATGGCACTTAGTGCAATTCTGAG | ACGTTGGATGTAGGCCTTAGTGTTTCCAGG |
| rs2157337 | 32504906 | ACGTTGGATGAGGCCTATAAGGAATGAGTG | ACGTTGGATGCAGAATGGACTTCAAAGTAC |
| rs981559 | 32505329 | ACGTTGGATGGCACATAGCAATATGGCTAC | ACGTTGGATGGGAACTAGAATTGCTACACAG |
| rs1987947 | 32505573 | ACGTTGGATGTTCCAAAGTAAGTGAGGCAC | ACGTTGGATGACAGTGACCTCAAAATTCCC |
| rs2395211 | 32506386 | ACGTTGGATGTGGTTTGGGAAGTGGGAGTG | ACGTTGGATGAACTGGGCTTCCTCAGCAGG |
| rs1894554 | 32506604 | ACGTTGGATGGGCAAGGATGATGTGTCTGC | ACGTTGGATGTTGGGTGTGATCTGCTCCAC |
| rs2395213 | 32506777 | ACGTTGGATGAGACACCTGCAAGCCTGCAG | ACGTTGGATGTCCATGCAGCAAGATCCAGG |
| rs2097440 | 32507071 | ACGTTGGATGTTCTGCCCAGGAGACTGTCTG | ACGTTGGATGTTGCCATGAGCAGCCTAGGTG |
| rs2097439 | 32507201 | ACGTTGGATGCTGCTGACACGAGTGGGAAC | ACGTTGGATGCTTTTACAGGCCTCAGAGGG |
| rs2006039 | 32540959 | ACGTTGGATGCAGATGATGAGGTAGGATGC | ACGTTGGATGTTACTGTGAACATCAGGGCC |
| rs1540307 | 32550985 | ACGTTGGATGGAGAGAGTCTATTCCCTTAG | ACGTTGGATGTAAACTAGTTCTCCTACTCC |
| rs707784 | 32566932 | ACGTTGGATGCCTCACCTTTCTGATTCCTG | ACGTTGGATGACAGAGCAAGATGCTGAGTG |
| rs2308665 | 32570455 | | |
| rs2395217 | 32575274 | ACGTTGGATGATGTTAGCCAGGATGGTCTC | ACGTTGGATGTAATCCCTGCACTTTGGGAG |
| rs1059544 | 32578551 | ACGTTGGATGGGTTCATAGTTCTCCCTGAG | ACGTTGGATGATGCTGGAGAACAGGACAGG |
| rs2647063 | 32588521 | ACGTTGGATGGACAGTAGCACATGTGAGTC | ACGTTGGATGTCTAGACACTGGTAACCCTG |
| rs2858860 | 32598186 | ACGTTGGATGCTGCAGACCTCACTCTATGG | ACGTTGGATGAGGAGCAGAGAAAAGTCCTG |
| rs2105899 | 32608060 | ACGTTGGATGCCAATCTCTGCTCAAGTGTG | ACGTTGGATGACTGGGCTTGAACAGTGATG |
| rs2040410 | 32620180 | ACGTTGGATGTACCTCATTAGGCAGTTGTG | ACGTTGGATGTGTCCTCCTTGGAAAATGAG |
| rs2213287 | 32622576 | ACGTTGGATGTAGAGACCTCCAGGCTATAG | ACGTTGGATGAAACCAGAGTCCCAACCTAC |
| rs1894385 | 32655597 | ACGTTGGATGGCTGCAGACATATCTAGGAG | ACGTTGGATGGCAAAGCTTCATTGAGGAGG |
| rs2395229 | 32664446 | ACGTTGGATGAAAGCGTGTGGGTGTTCTAG | ACGTTGGATGTGGTAAGCATCACTGTCTCC |
| rs1360 | 32666380 | ACGTTGGATGTCTTCTGGTTTGGTGAGTGC | ACGTTGGATGAAGGGTCACTATATCTGCCC |
| rs1064173 | 32666475 | ACGTTGGATGATATTCTCAGGCCACTGCAC | ACGTTGGATGAGGAGGTAGAAGATCAACTC |
| rs1056316 | 32666490 | ACGTTGGATGGGGTTGTACCTTGAAAAGAC | ACGTTGGATGCATGAATGATGCGACAACTG |
| rs1762 | 32666522 | | |
| rs2647027 | 32674779 | ACGTTGGATGAGTACTGTCCCTAGTCACTG | ACGTTGGATGCAG1TCCTCATGGACATATC |
| rs2395231 | 32676429 | ACGTTGGATGTTCATAGAGCATGAGGAGCC | ACGTTGGATGACATTTGAGGGCAAATGAGG |
| rs2647015 | 32677576 | ACGTTGGATGAATGAAGATGACAGGCAGAG | ACGTTGGATGACTCACAGAAGCCAAAGAAG |
| rs2157051 | 32682878 | | |
| rs2894283 | 32692730 | ACGTTGGATGCTGTTCATCTCTATTGACTTG | ACGTTGGATGCCAAAGCATTTAATGGTTTAG |
| rs2894284 | 32693263 | ACGTTGGATGAGAACCAAACCTTCACTTGG | ACGTTGGATGGTTATGGGTGTTGTTTAGCC |
| rs2858888 | 32696820 | ACGTTGGATGCTTCAGGGCAAAAGACAATG | ACGTTGGATGCCCCTTAAGATGGTCTAATAG |
| rs2859112 | 32700110 | | |
| rs2859091 | 32703147 | ACGTTGGATGTGACTTCCTTTTCTCCCAGG | ACGTTGGATGAACACATCAGAAGGCACACC |
| rs2051599 | 32711686 | ACGTTGGATGAGGAATGTTCTCTGGAGCTG | ACGTTGGATGGACCCTTGGGAAATTTCTAC |
| rs2395252 | 32713659 | ACGTTGGATGAAAGCAGAAGGCCCTGCTGAG | ACGTTGGATGACATCACTCTACTGGCCCAG |
| rs2071800 | 32716486 | ACGTTGGATGTGGGCACTGTCTTCATCATC | ACGTTGGATGTCATAAGAGCCCTTGGTGTC |
| rs2395253 | 32717006 | ACGTTGGATGAGCTTTCCTCTCCCCTTCTC | ACGTTGGATGCTGGCTTCCATTTCTTTTCC |
| rs2213572 | 32722145 | ACGTTGGATGGACAACAAAAAAAATACTTCT | ACGTTGGATGTTTCAGTGAGATCCTGGGTTA |
| rs1573649 | 32728253 | ACGTTGGATGGGATCTGCAGAGCCATCTTC | ACGTTGGATGTGAGCTGTGTTGACTACCAC |
| rs1573647 | 32728610 | ACGTTGGATGCCTCACTTAATTTGCCCTAC | ACGTTGGATGGAAGATTGAATGGCTTAGGG |
| rs2857210 | 32738722 | ACGTTGGATGGAAGCCTTCAATGTTACAGG | ACGTTGGATGACTCCAGAAGAGTAGAGTGG |
| rs719654 | 32749097 | ACGTTGGATGGTGACACTAATAACCCAAGG | ACGTTGGATGGTAGTGAACTTCCATGCAGG |
| rs2157080 | 32758398 | ACGTTGGATGAGAGGACACAGTCATCTCAG | ACGTTGGATGCGAGTAGGTACTCTCATTGG |
| rs2621343 | 32771751 | ACGTTGGATGTATTCCACTCCCAACTCCTG | ACGTTGGATGGCGGATTCCTAATTCTGAGG |
| rs2857107 | 32782267 | ACGTTGGATGGAGTGTTAAAGGTAGAAGCC | ACGTTGGATGAAGTGTATCCCATTTTTTCC |
| rs241447 | 32793449 | | |
| rs2127673 | 32809315 | ACGTTGGATGAACTGTCGACGTCACACGAC | ACGTTGGATGTCTGAAGCTGCACCTGGAGG |
| rs1871668 | 32825643 | ACGTTGGATGATACTAGTAGGATCTCAGGC | ACGTTGGATGGAAACAACTCCAGGCATTTG |
| rs1383267 | 32830393 | ACGTTGGATGCTCGGTTCTAACCAAGTAGG | ACGTTGGATGATGTTACCTTGGCGAAAGGC |
| rs241415 | 32839637 | ACGTTGGATGTTTCCTCAATAGGTGTAGAC | ACGTTGGATGCTATGAACAATTCTACACAC |
| rs1029295 | 32853431 | ACGTTGGATGGAATTCACAGGCTTTTAGCC | ACGTTGGATGAGGCTTAATGATGAGAGGTG |
| rs241404 | 32862944 | ACGTTGGATGAATGTCATATGCCTCCTCCC | ACGTTGGATGTGCAACTATCTGGACACATG |
| rs2187688 | 32868648 | | |
| rs154985 | 32877112 | ACGTTGGATGACCTGTTGGGAAATGTAGGC | ACGTTGGATGCCATGAGTGAGGATTCCAAG |
| rs151722 | 32892567 | ACGTTGGATGCTGGCCTGAGTTTTGATAAG | ACGTTGGATGGGCAAGCTACATAATGGAAG |
| rs151719 | 32900606 | ACGTTGGATGAGGACACATGGGAGATCTAG | ACGTTGGATGTAACCTCCAGTGGATCCATC |
| rs10679 | 32913451 | ACGTTGGATGTCCAAACAGAGGATGCTCAG | ACGTTGGATGTCCCAGAGACTTCTTCTACC |
| rs1431394 | 32926004 | ACGTTGGATGTATGCACTAACCCATCAGCG | ACGTTGGATGCTTCTTTTCTACTGTCCAGG |
| rs206787 | 32938141 | ACGTTGGATGTGAGGCAGGAGGTCAGCAC | ACGTTGGATGTCCGGACCGGAACCGCATCT |
| rs2567267 | 32948944 | ACGTTGGATGGACTTGTTTTTCATGGCGTAG | ACGTTGGATGCTCCAGCCTGGAGTCTTTAAA |
| rs188245 | 32955171 | ACGTTGGATGATCACTGCCTTTGGTGTTGC | ACGTTGGATGACTCCCTGGCCAAATGATTG |
| rs3135332 | 32969029 | ACGTTGGATGATGTTTGATAGCAGACTGGG | ACGTTGGATGCCTCTCTTCTAGCTACTTTG |
| rs419434 | 32988697 | ACGTTGGATGGGCAGTGTGAACTAAGAGTG | ACGTTGGATGAGTGTCTCCAACTATGTGGC |
| rs3128942 | 32998935 | | |
| rs663310 | 33009016 | ACGTTGGATGGTCAGCCTCTGTATAAGGAC | ACGTTGGATGTAGGAGAGAGCCAAATCCAG |
| rs377572 | 33017193 | ACGTTGGATGTTTCCCACCTCCACAGTTTG | ACGTTGGATGAAAGCTGAGAGAACCCACAG |
| rs412735 | 33026279 | ACGTTGGATGTCCAGTCAAAGAGTGAACCC | ACGTTGGATGCATATGGAAGGGTGTGCAAG |
| rs2308935 | 33038580 | ACGTTGGATGCTCCTCTTTACATTCCCACC | ACGTTGGATGTAAAGTCTCTGCGTTCTGGC |
| rs2071351 | 33045675 | ACGTTGGATGTTACTGATGGTGCTGCTCAC | ACGTTGGATGAATTGTTCCCTGAGCCAGAC |
| rs3117227 | 33058913 | ACGTTGGATGCACAGTTCCCTAACGAGAAG | ACGTTGGATGGGTACCCCTTGATAACCATC |
| rs2144014 | 33067793 | ACGTTGGATGTGTGTAGATCTCTAGCGAGG | ACGTTGGATGAAGCCTCCAAGAAATTTGGG |
| rs3130216 | 33079418 | ACGTTGGATGAGAGACTGAGTTCAGTGTGG | ACGTTGGATGACTGAGACCACCCATCATAC |
| rs1883414 | 33089789 | ACGTTGGATGCAATCCATTGGTGTAACAGG | ACGTTGGATGAGATTACCACCTATAGACTG |
| rs3129272 | 33099767 | ACGTTGGATGTCCACTCCACAGATGATGAG | ACGTTGGATGTGTTCTTCCTAGAGGCACAG |
| rs2294479 | 33101481 | ACGTTGGATGACCTCAGTTTTGCATCCTGC | ACGTTGGATGTCCATTTTTGTCCCCTGGAC |
| rs2294478 | 33102058 | ACGTTGGATGTTTTGTCCCCCATCCCTTTC | ACGTTGGATGACAAGAAGGAGATGGTCTGG |
| rs2015610 | 33110987 | ACGTTGGATGCTCAGTGATTGGCACAAGTG | ACGTTGGATGGCCTAAAGGTTTCTCTGTAC |
| rs3130153 | 33119103 | ACGTTGGATGGGTACCATCAGAATACTGTC | ACGTTGGATGTTCACGGCTTGACTCAATGG |
| rs3129206 | 33128803 | ACGTTGGATGCTAAGGGAAGGAGAACTCTC | ACGTTGGATGAAGGTGGCACTGATTCTAGC |
| rs734181 | 33133246 | ACGTTGGATGTAGACTGGGCTATGTAGCAC | ACGTTGGATGATGGCTCCAGTTTCTGACAC |
| rs2076311 | 33148710 | ACGTTGGATGATCCCACCCCCATTCTTATC | ACGTTGGATGAAGAAGGCAAGAGCAGGAAG |
| rs2855457 | 33158566 | ACGTTGGATGCCAAGCCAGTCAACATTTTC | ACGTTGGATGTTGTCTCATTTCCAGAGCCC |
| rs2855433 | 33161160 | ACGTTGGATGGGTTTAGGAGATGAGTTGGG | ACGTTGGATGATCCACAGATGTGTGCTCAG |
| rs2982275 | 33168406 | ACGTTGGATGCCTTCTTCTGTGTCTCCATC | ACGTTGGATGAAGTGGGTGTTTTGACCAAG |
| rs421446 | 33178124 | ACGTTGGATGACTGTGTATGCGTGACACTC | ACGTTGGATGTGCAGAACCAGTGGAAAGGG |
| rs1704996 | 33185888 | | |
| rs213213 | 33186822 | | |
| rs213194 | 33198941 | ACGTTGGATGGGTGGAGAGATGTGATTTCC | ACGTTGGATGTATACCGTCCAATAGGAGGC |
| rs213224 | 33209549 | ACGTTGGATGATCCAAGCACTTTGGGAAGC | ACGTTGGATGTGTTTTGCCATGTTGGCCAG |
| rs213225 | 33211713 | | |
| rs213226 | 33212398 | ACGTTGGATGCCTGGCTTGCTTTCTTCTTG | ACGTTGGATGGCAGCATGGTTTTGTACAAG |
| rs105445 | 33220331 | ACGTTGGATGTGAGGGAACGCATAGCGCAG | ACGTTGGATGTTAACTGACCTCGCCCTTGC |
| rs1269806 | 33229599 | ACGTTGGATGAACACAGAAAGACCCTCATC | ACGTTGGATGGAGTTCTTTGCATCATCTAC |
| rs213202 | 33235198 | ACGTTGGATGTCACCATGAGTTTCACCACC | ACGTTGGATGGCTCTAAGCATCATTGTGGG |
| rs2231260 | 33249259 | ACGTTGGATGAGGCCACTGCTCCTCTGATAC | ACGTTGGATGTGCCTCTTCTGTACTTGGGC |
| rs464865 | 33259080 | ACGTTGGATGGCAGCTTATGCAAGAGTGAC | ACGTTGGATGAAAAGAAACCGCACCGCTAC |
| rs1014779 | 33278611 | ACGTTGGATGGCCAAGGACGGCTTGGAATA | ACGTTGGATGACGAACACTAACGATGGCTG |
| rs1061783 | 33284571 | ACGTTGGATGCCTTTATCTCTGTGGACTTG | ACGTTGGATGAGTTCTGGAAATACCTTGGG |
| rs3130016 | 33308361 | ACGTTGGATGAGTGGCTCATGCCTGTAATC | ACGTTGGATGCTCCTGACTTTAGGTGATCC |
| rs3130267 | 33318929 | ACGTTGGATGACCATGTGGAGCAAAAAGGC | ACGTTGGATGAACACGTTGGATGTTGTGCG |
| rs1265492 | 33322652 | ACGTTGGATGAGGCCCTCAAAATCACAAAC | ACGTTGGATGACGTGTTAGATGTGAGGAAG |
| rs3117323 | 33327502 | ACGTTGGATGCACGTGTTTATCTGCTGACC | ACGTTGGATGTTGGGTGTTTCTCAGAGAGG |
| rs211450 | 33335443 | ACGTTGGATGATGTCTGGAACTGGACCCTG | ACGTTGGATGATGGTGCCCGTACGGTTTGG |
| rs211447 | 33346185 | ACGTTGGATGTTTTGTCGACCCTGCACTTG | ACGTTGGATGGCCATAAGATCAGATGAGCC |
| rs465877 | 33358320 | ACGTTGGATGAGGAGAATGGCATGAACCCC | ACGTTGGATGTTTTTGAGACAGAGTCTGGC |
| rs456993 | 33360326 | ACGTTGGATGCCAACCTTTGATATCCTGGG | ACGTTGGATGTCCCCCTTTACCTTCCATTG |
| rs211457 | 33367887 | ACGTTGGATGCCAGACAGCATGAACAGAAG | ACGTTGGATGAAGTCCAGCTTTTCCCCTAG |
| rs3106193 | 33377160 | ACGTTGGATGCCGAAGTGTTCGGATTATAG | ACGTTGGATGGAAAAGGTGACTAAAAGGTC |
| rs1705003 | 33388001 | ACGTTGGATGAGTAGAAGAAGGACCACCTG | ACGTTGGATGATTGGTCAAGTCTCCCATGG |
| rs2076775 | 33396500 | ACGTTGGATGTCACCCTTTCTCTTTCCTCC | ACGTTGGATGTGGGAGTCTGATGGACTTTC |
| rs453590 | 33405670 | ACGTTGGATGAACTCACTTTCCTCCCTAGG | ACGTTGGATGAAGCCCAACAAGGTATTGGG |
| rs3119027 | 33416775 | ACGTTGGATGAGCATCATTGGCAGGTGAGG | ACGTTGGATGAGTTTGGGGATGGGAGTCAG |
| rs3119025 | 33425122 | ACGTTGGATGAGAACTTTCCCTCTAGCCTG | ACGTTGGATGATCGAGTTCCCACAGCATAG |
| rs1755047 | 33433266 | ACGTTGGATGTCTCTCTTTCCCTGTAGCTC | ACGTTGGATGATGCCCAAGCCAAGAAAGAG |
| rs1755049 | 33440490 | ACGTTGGATGTTCAGCCTCTGGTGTAGCTG | ACGTTGGATGTAACACGGTGAAACCCCGTC |
| rs2772381 | 33456461 | | |
| rs210190 | 33466198 | ACGTTGGATGATAGTGCTCACTGGCTGAAG | ACGTTGGATGACTATCACAGGAAGCAGTCG |
| rs1755038 | 33467280 | ACGTTGGATGATGATGGCAGCAGCCACTGC | ACGTTGGATGCCTCCAGTAGCATGTAAGGC |
| rs769051 | 33476004 | ACGTTGGATGACCTCTGCAGACTTAGACTG | ACGTTGGATGCGCATAAAGTAGAGGGACTC |
| rs210180 | 33487120 | ACGTTGGATGAAACCCACACCTGCAGTGAG | ACGTTGGATGAGGCTTTCCACACACTCCTG |
| rs210184 | 33488865 | ACGTTGGATGTCCTTTCCTCTGGGTTAGCC | ACGTTGGATGTGTCTTCACCACAGGCAGTG |
| rs429789 | 33497846 | ACGTTGGATGTTTGAGATGGAGTCTCGCTC | ACGTTGGATGAGGAGAATTGCTTGAACCCG |
| rs210196 | 33509584 | ACGTTGGATGAAGCAGCTGGGAAAGAAACG | ACGTTGGATGATGACAGTGCTTCCAGAGAG |
| rs210203 | 33513090 | ACGTTGGATGAGGGCAAATGAAATCTGTCC | ACGTTGGATGTCTCTGTGCCTATGCATACC |
| rs210158 | 33521419 | ACGTTGGATGCCCTTGCCTTATCTTTCTTG | ACGTTGGATGCACTAGGGAAATGGTTGTGC |
| rs210169 | 33526576 | | |
| rs210131 | 33537576 | ACGTTGGATGAGAGGAGGAAGAGCTGAAAG | ACGTTGGATGATTCATGTAAGGCACGGACC |
| rs210133 | 33538658 | ACGTTGGATGGGCTCACCAACCTTCTCATG | ACGTTGGATGTAACTGGATAAGCTGCCCTC |
| rs210132 | 33538781 | ACGTTGGATGTTTCAGAGACACCAGACATG | ACGTTGGATGTTGCTAAAGTCTCAGGTGGG |
| rs210135 | 33542803 | ACGTTGGATGAGAACCCTCCAGATGAACTC | ACGTTGGATGACTACAGGGCTTAGGACTTG |
| rs513349 | 33543830 | ACGTTGGATGCTTCCTCGGGTTCCTATATC | ACGTTGGATGGAGAAACAAGGTGGTCACAG |
| rs210139 | 33545520 | ACGTTGGATGTCTAAGACATGAGTGCTGGG | ACGTTGGATGTTTTATGTTGGGCTCCCACC |
| rs210141 | 33548935 | ACGTTGGATGCCAAGAGCTCTCAAGAAGGG | ACGTTGGATGTAACAAGGCCTTGCCCCTAG |
| rs210145 | 33549551 | ACGTTGGATGTGGCCTAAATTCCCGGTGAG | ACGTTGGATGAACATCCCTAGACTGGGTCC |
| rs2894350 | 33556912 | ACGTTGGATGTCTAAATTCAGGACCCTGGC | ACGTTGGATGCAGAGAGACTGATGGAGAAG |
| rs396746 | 33558906 | ACGTTGGATGTTTGGCCTCATTGTTGGCTG | ACGTTGGATGAAGACCTCAGATAGACTGGG |
| rs210162 | 33561565 | ACGTTGGATGTTCGACTCTTCCCGGACTCC | ACGTTGGATGTTCCCCCTTGCCCCATATGAG |
| rs210120 | 33576523 | ACGTTGGATGGCTCAGCTTAAATGTCTCCC | ACGTTGGATGGAGAGCAGAAAAGAGAGAGG |
| rs407415 | 33581077 | ACGTTGGATGAACCCACCTTTCTTGTTGGC | ACGTTGGATGTCTTCCTTTCTCCAGACTCC |
| rs2395451 | 33589837 | ACGTTGGATGGGACTGGATGAGGTTTTTTA | ACGTTGGATGGCTAAGTACCGTGTTTTACAG |
| rs1536043 | 33594867 | ACGTTGGATGTCCTCCACTTTTTGTTGGCC | ACGTTGGATGTTATCCCTTACCCTAGGTGG |
| rs1536042 | 33620471 | ACGTTGGATGAGTCACTTGAATGGGCTCAG | ACGTTGGATGTAACAGCCCTTATAGGGTGG |
| rs999943 | 33626118 | ACGTTGGATGTATAGCTGTGGACTGGGCTG | ACGTTGGATGAGGAAGGAAGCCTGTTGCAG |
| rs2229634 | 33640290 | ACGTTGGATGGCCATGCACAGGAAAATCAG | ACGTTGGATGTACCAGCTGAAGCTCTTTGC |
| rs753890 | 33654495 | ACGTTGGATGTCACGTGGTCCTTTCATCAC | ACGTTGGATGTGAGAAGAGTGGGCATGATG |
| rs658087 | 33667130 | ACGTTGGATGAAGCAGGCTTTTTGCCTCTC | ACGTTGGATGGGGAGGAAGCCAAAAATAGC |
| rs2281829 | 33677752 | ACGTTGGATGGCTGTAGGAAACACGTGTTC | ACGTTGGATGTCCTCTTACACCCCATATCC |
| rs1555965 | 33679261 | ACGTTGGATGCTTTACACTTTGGGCCAGTG | ACGTTGGATGTGGCCCAGGTTATACGATAC |
| rs549652 | 33688213 | ACGTTGGATGAATGTCATCAGGAAGCCCTG | ACGTTGGATGCCTGCGTGGTTTAAAAGCTC |
| rs630792 | 33692421 | ACGTTGGATGTCTCCGATGAGCAGCATTAG | ACGTTGGATGGCTGAACAAATCAGCTCTGG |
| rs597723 | 33696190 | ACGTTGGATGCCTTCCATCCCTCCAAATTC | ACGTTGGATGCACACTTCCCTCTCACTGTG |
| rs608971 | 33703990 | ACGTTGGATGTAGAATCCGGCGTGTATGTG | ACGTTGGATGTTCTGATTCACAGGTCTGGC |
| rs568901 | 33712149 | ACGTTGGATGAAGTCGACGCCCATTCTGAC | ACGTTGGATGAGACAGCCAGAGCAACTCAG |
| rs570749 | 33712340 | ACGTTGGATGAGCACTTCGTTAGACACTGC | ACGTTGGATGTTTCAGAAGCTGCACCTGAC |
| rs530614 | 33716891 | ACGTTGGATGTCTCTCCTCCTCTTTGTCCC | ACGTTGGATGGACTGGCATGTCCAGCTGTC |
| rs2395449 | 33730616 | ACGTTGGATGAGACTACGCATCCTCTTCTC | ACGTTGGATGTGCAAACCTCTCAGAGTCTC |
| rs755496 | 33736487 | ACGTTGGATGCCTGTGCCCTTATGATTCTG | ACGTTGGATGAACCAATCCCTGGGATGGAG |
| rs755497 | 33736530 | ACGTTGGATGAAGCATGGTTCTGTGCCCTG | ACGTTGGATGAACCAATCCCTGGGATGGAG |
| rs943473 | 33745761 | ACGTTGGATGATGCTGAGAGCTCACCCTTG | ACGTTGGATGTTTGCATCTGTGGAGAGCTC |
| rs943474 | 33751945 | ACGTTGGATGACTTTGAAACTGAACTGAAG | ACGTTGGATGCTTTGAAGTAGGAGGATCTG |
| rs943475 | 33752089 | ACGTTGGATGAACTGGATTGGGAAAGGGAG | ACGTTGGATGTTTCCCACTAGGACTCTTCC |
| rs943479 | 33753874 | ACGTTGGATGTTAGTTACACTGCTGCTGGC | ACGTTGGATGGACTCGGCTTCTGAATATGC |
| rs2395402 | 33755534 | ACGTTGGATGCCCTGCATTGACTGTCTTAC | ACGTTGGATGAACTATGACACACCCGAAGC |
| rs2013365 | 33765798 | | |
| rs2894342 | 33776504 | ACGTTGGATGAATAGCTGTGTGACCTTGGG | ACGTTGGATGTTACAACAGCCCTGAGGTTC |
| rs1547668 | 33777490 | ACGTTGGATGTAGTGGCTGTTTCTCTCCTG | ACGTTGGATGATATCCGTGGCAATTCCCAC |

Genotyping HLA Loci

The invention features a novel method of genotyping Human Leukocyte Antigen (HLA) genes using patterns of neighboring single nucleotide polymorphisms (SNPs). The SNP-based method is an improvement over existing hybridization-based techniques, as it allows quick and inexpensive genotyping of the HLA loci. This method does not directly assess the intra-gene variation, as is done by all other current methods for HLA genotyping, but rather defines HLA genotypes by studying the neighboring extra-genic variation(s) which, due to LD patterns, is conveniently linked to the HLA loci. By “extra-genic” herein is meant outside or in the neighboring region(s) of the HLA allele to be genotyped. Identification of the correlation of this extra-genic variation to the HLA gene alleles allows for the discovery and utilization of surrogate markers for HLA genotypes.

One aspect of the invention provides a method of genotyping an HLA gene, such as for example an HLA-A or an HLA-DRB1 gene. The method comprises determining the nucleotide present at one or more extra-genic SNP sites, wherein the SNP is associated with an HLA genotype. For example, to genotype the HLA-A allele, an extra-genic SNP to be assessed can be rs2517862, rs1655930, rs1616549, rs376253, rs1961135, rs2517706, rs2517701, rs2517699, rs435766, rs410909, rs2394255, rs1264807, rs2530388, rs356963, rs2286405, rs2240619, rs3129012, rs259938, or any combination thereof. Another example involves genotyping the HLA-DRB allele, wherein an extra-genic SNP to be assessed can be rs742697, rs523627, rs3129960, rs2395163, rs2395165, rs983561, rs2239804, rs2213584, rs2395182, rs2858860, rs3129907, rs1059544, rs1987529, or any combination thereof.

Nomenclature and designations of the HLA alleles have been described by Marsh et al., Tissue Antigens (2002) 60:407-464. A summary of HLA-A, -B, -C, -DRB1/3/4/5, -DQB1 alleles and their association with serologically defined HLA-A, -B, -C, -DR and -DQ antigens is provided by Schreuder et al., Tissue Antigens (2001) 58:109-140.

Methods of determining or analyzing SNPs are known in the art. For example, to detect any particular SNP in target DNA sample, e.g., a DNA sample from a subject to be tested, preferable a human subject, one can employ any of the known procedures in the art. For example, two distinct types of analysis and seven procedures are described in U.S. patent application Ser. No. 10/213,272, Publication No. 20030170665, incorporated herein by reference in its entirety. The first type of analysis is sometimes referred to as de novo characterization. This analysis compares target sequences in different individuals to identify points of variation, i.e., polymorphic sites. By analyzing a group of individuals representing the greatest variety patterns characteristic of the most common alleles/haplotypes of the locus can be identified, and the frequencies of such populations in the population determined. Additional allelic frequencies can be determined for subpopulations characterized by criteria such as geography, race, or gender. The second type of analysis determines which form(s) of a characterized polymorphism are present in individuals under assessment. There are a variety of suitable procedures:

1). Allele-Specific Probes

The design and use of allele-specific probes for analyzing SNPs is described by e.g., Saiki et al., Nature 324:163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Some probes are designed to hybridize to a segment of target DNA such that the polymorphic site aligns with a central position (e.g., in a 15 mer at the 7 position; in a 16 mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2). Tiling Arrays

The SNPs can also be identified by hybridization to nucleic acid arrays. Subarrays that are optimized for detection of a variant forms of a precharacterized polymorphism can also be utilized. Such a subarray contains probes designed to be complementary to a second reference sequence, which is an allelic variant of the first reference sequence. The inclusion of a second group (or further groups) can be particular useful for analyzing short subsequences of the primary reference sequence in which multiple mutations are expected to occur within a short distance commensurate with the length of the probes (i.e., two or more mutations within 9 to 21 bases).

3). Allele-Specific Primers

An allele-specific primer hybridizes to a site on target DNA overlapping an SNP and only primes amplification of an allelic form to which the primer exhibits perfect complementarily. See Gibbs, Nucleic Acid Res. 17, 2427-2448 (1989). This primer is used in conjunction with a second primer which hybridizes at a distal site. Amplification proceeds from the two primers leading to a detectable product signifying the particular allelic form is present. A control is usually performed with a second pair of primers, one of which shows a single base mismatch at the polymorphic site and the other of which exhibits perfect complementarily to a distal site. The single-base mismatch prevents amplification and no detectable product is formed. The method works best when the mismatch is included in the 3′-most position of the oligonucleotide aligned with the polymorphism because this position is most destabilizing to elongation from the primer.

4). Direct-Sequencing

The direct analysis of the sequence of any samples for use with the present invention can be accomplished using either the dideoxy-chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

5). Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

6). Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence difference between alleles of target sequences.

7). Single Base Extension

An alternative method for identifying and analyzing SNPs is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997)), uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently-labeled dideoxyribonucleotides (ddNTPs) in dye-terminator sequencing the effect of mtDNA D-loop sequence polymorphism on milk production, each cow was the next generation of the herd.

TABLE 3 shows exemplary extra-genic SNPs that correspond to HLA-A alleles and can be used in genotyping HLA-A alleles. The SNPs and HLA-A allele are lined up in each row of the table from the left to the right according to their respective positions on chromosome 6. The percentage numbers on the right column represent the likelihood of the identity of a particular HLA-A allele when the exemplary SNPs are determined to be as shown in the respective rows. For example, in row 1, the HLA-A allele has a 100% likelihood to be HLA-A*2402, when the 18 SNPs listed are determined to be the respective nucleotides as shown in row 1. Take row 5 as another example, the HLA-A allele has a 92% likelihood to be HLA-A*101, when the 18 SNPs listed are determined to be the respective nucleotides as shown in row 5. The allele-type determinative SNPs between HLA-A*2402 and HLA-A*101 include: rs2517862, rs1655930, rs376253, rs1961135, rs2517706, rs1264807, and rs3129012.

TABLE 3

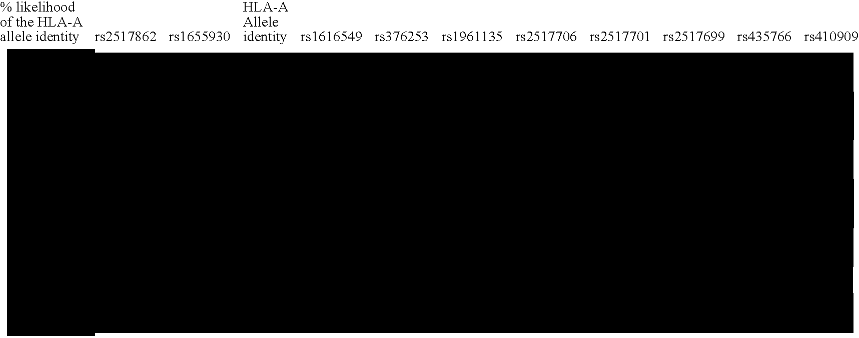

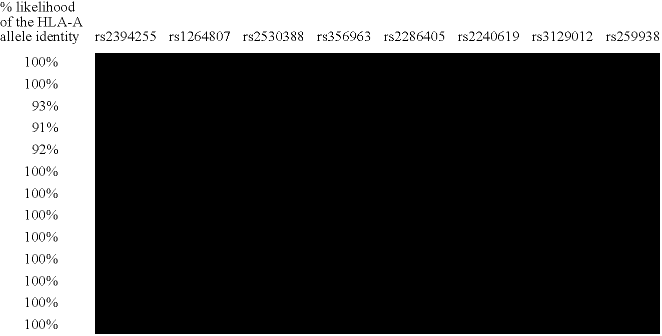

Legend: 1 = A; 2 = C; 3 = G; 4 = T

TABLE 4

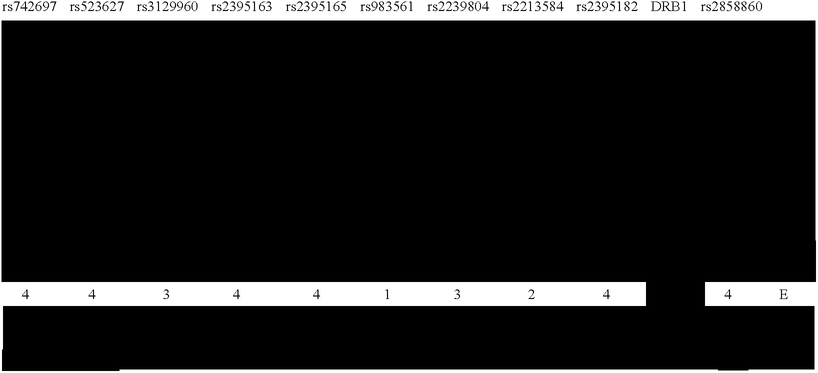

Legend: 1 = A; 2 = C; 3 = G; 4 = T

The above table shows exemplary extra-genic SNPs that correspond to HLA-DRB1 alleles and can be employed to genotype HLA-DRB1 alleles. Again the relative positions of the SNPs and the HLA-DRB1 on human chromosome 6 are shown (from left to right in each row). The letters on the right-most column are arbitrarily assigned to the SNP-haplotype alleles as shown on each row. For example, row 1 corresponds to SNP-haplotype allele J, with the extra-genic SNPs determined to be the nucleotides as shown in this row. Where ambiguity exists, e.g., row 4, where the SNP-haplotype could be B or W, and this ambiguity may be resolved by determining an additional SNP: rs3129907. And if rs3129907 is 1 or A, the SNP-haplotype allele will be B, and if rs3129907 is 3 or 6, the SNP-haplotype allele will be W. Similarly, row 6, the SNP-haplotype allele can be ascertained by determining the SN-P rs1059544 (2 or C will correspond to SNP-haplotype allele U, and 4 or T will correspond to SNP-haplotype allele V). Also, row 11, the SNP-haplotype allele can be ascertained by determining the SNP rs1987529 (3 or 6 will correspond to SNP-haplotype allele K, and 1 or A will correspond to SNP-haplotype allele T). Also, Similarly, row 14, the SNP-haplotype allele can be ascertained by determining the SNP rs1987529 (1 or A will correspond to SNP-haplotype allele G, and 3 or G will correspond to SNP-haplotype allele H). Similarly, row 16, the SNP-haplotype allele can be ascertained by determining the SNP rs2395165 (4 or T will correspond to SNP-haplotype allele A, and 2 or C will correspond to SNP-haplotype allele R).

Next, FIG. 4B shows the percentage of a particular SNP haplotype allele that bears the indicated HLA allele. For example, SNP-haplotype allele J, having the SNPs as shown in row 1 above, corresponds to an HLA-DRB1 allele that has a 100% likelihood to be HLA-DRB1*1302. Take row 2 as another example, SNP-haplotype allele N, having the SNPs as shown in this row, corresponds to an HLA-DRB1 allele that has a 92.6% likelihood to be HLA-DRB1*1501.

The invention further features a method of predicting or assisting in the prediction of the likelihood or probability of development of a disease, particularly an MHC-linked disease, in a subject, preferably a human subject. The method comprises genotyping an HLA gene in the subject to be tested by determining the nucleotide present at one or more extra-genic SNP sites, wherein the SNP is associated with an HLA genotype. MHC-linked diseases include, but are not limited to, ankylosing spondylitis, Behcet Syndrome, common variable immunodeficiency, Goodpasture Syndrome, psoriasis, inflammatory bowel disease, insulin-dependent diabetes mellitus (type 1), multiple sclerosis, myasthenia gravis, pemphigus vulgaris, rheumatoid arthritis, systemic lupus erythematosus. Identification of an HLA genotype in the subject which is associated with a disease is indicative that the subject has a greater likelihood of developing the disease. For example, HLA-DRB1*1101 genotype is associated with pemphigoid diseases, as discussed above.

The invention further features a method of predicting or assisting in the prediction of the likelihood or probability of development of a disease, particularly an autoimmune disease, in a subject, preferably a human subject. The method comprises genotyping an HLA gene in the subject to be tested by determining the nucleotide present at one or more extra-genic SNP sites, wherein the SNP is associated with an HLA genotype. Identification of an HLA genotype in the subject which is associated with a disease is indicative that the subject has a greater likelihood of developing the disease. For example, HLA-DR2 haplotype is linked or associated with multiple sclerosis and lupus. Examples of autoimmune diseases grouped based on main target organs include, but are not limited to:
1) Nervous System: multiple sclerosis, myasthenia gravis, autoimmune neuropathies such as Guillain-Barré, autoimmune uveitis;
2) Gastrointestinal System: Crohn's Disease, ulcerative colitis, primary biliary cirrhosis, autoimmune hepatitis;
3) Blood: autoimmune hemolytic anemia, pernicious anemia, autoimmune thrombocytopenia;
4) Endocrine Glands: Type 1 or immune-mediated diabetes mellitus, Grave's Disease, Hashimoto's thyroiditis, autoimmune oophoritis and orchitis, autoimmune disease of the adrenal gland;
5) Blood Vessels: temporal arteritis, anti-phospholipid syndrome, vasculitides such as Wegener's granulomatosis, Behcet's disease;
6) Multiple Organs Including the Musculoskeletal System (These diseases are also called connective tissue (muscle, skeleton, tendons, fascia, etc.) diseases.): rheumatoid arthritis, systemic lupus erythematosus, scleroderma, polymyositis, dermatomyositis, spondyloarthropathies such as ankylosing spondylitis, Sjogren's syndrome;
7) Skin: psoriasis, dermatitis herpetiformis, pemphigus vulgaris, vitiligo.

A further aspect of the invention provides a method of predicting or assisting in the prediction of the likelihood of developing an immune response in a subject, preferably a human subject. An immune response may be developed against an infecting organism or agent. Alternatively, an immune response may comprise a host-graft response, e.g., rejection of organ transplants. The method comprises genotyping an HLA gene in the subject to be tested by determining the nucleotide present at one or more extra-genic SNP sites, wherein the SNP is associated with an HLA genotype. The method may also comprise separately genotyping an HLA gene in a host (e.g., a blood or organ recipient or donee) and the same HLA gene (or the corresponding HLA gene) in a graft (e.g., a blood or organ donor) by determining the nucleotide present at one or more extra-genic SNP sites in the host and the graft, wherein the SNP is associated with an HLA genotype. Genotyping an HLA gene in a host may involve assessing more, fewer, or the same extra-genic SNPs as compared the extra-genic SNP(s) to be assessed in a graft.

In preferred embodiments of the invention, more than one extra-genic SNP, more preferably more than three extra-genic SNPs, more preferably more than five extra-genic SNPs, and more preferably more than seven extra-genic SNPs are determined in order to determine the genotype of an HLA allele.

An exemplary method of determining whether or not a host and a graft have the same HLA alleles or immune-compatible HLA alleles may include:
a) determining the HLA allele in the host (or graft) by ascertaining the nucleotide present at one or more extra-genic SNP sites or any other method;
b) selecting extra-genic SNPs to be assessed in the graft (or host) based on the HLA allele identity as determined in a);
c) assessing the selected extra-genic SNPs to identify the HLA allele genotype.

For example, if a host is determined by a method of the invention or any other method to have an HLA-A*101 allele (e.g., having SNP-haplotype as shown in row 5 of TABLE 3 above), only rs2517862 and/or rs1655930 need to be assessed to ascertain that a graft does not have HLA-A*101. Based on the information in TABLE 3, one can optimize the selection of SNPs to be assessed.

All publications, patents, patent applications and information from databases cited above are hereby incorporated by reference in their entirety for all purposes to the same extent as if each individual publication or patent application were specifically and individually indicated to be so incorporated by reference.

The invention is now further described in the following non-limiting examples.

### EXEMPLIFICATION

### Example 1

### Materials and Methods

DNA Samples

Samples were obtained from the Coriell Cell Repository and drawn from the collection of Utah CEPH pedigrees of European descent. One hundred thirty-six independent, grandparental chromosomes were used for haplotype construction. Of these chromosomes, 96 were in common with Gabriel et al. (2002) and, therefore, were used for comparison with the genome-wide LD structure. Identifiers for all individuals can be found at the Inflammatory Disease Research Group (IDRG) Website.

Genotyping and Data Checking

All SNPs for which genotyping was attempted were publicly available at the dbSNP Web site. SNPs were selected mainly to achieve a desired spacing (1/20 kb); however, SNPs with more than one submitter were preferentially chosen. SNP primers and probes were designed in multiplex format (average fivefold multiplexing) with SpectroDESIGNER software (Sequenom). A total of 435 assays were designed. Assays were considered successful and genotype data were included in the analyses described herein if they passed all of the following criteria: (1) a minimum of 75% of all genotyping calls were obtained, (2) markers did not deviate from Hardy-Weinberg equilibrium, and (3) markers had no more than one Mendelian error. These criteria defined 201 successful assays. Genotype calls for successful markers were then set to zero for any single Mendelian error. All of these working assays had minor allele frequencies 15%, and 89% of these assays had minor allele frequencies 110%. Overall, for successful markers, 97.6% of all attempted genotypes were obtained. The entire list of SNP assays, as well as detailed genotyping information, can be found at the IDRGWeb site. Four-digit HLA types were determined for HLA-A, HLA-B, HLA-C, HLA-DRB1, HLA-DMB1, HLADQA1, HLA-DQB1, HLA-DPA1, and HLA-DPB1, as described elsewhere (Begovich et al. 1992; Carrington et al. 1994, 1999; Moonsamy et al. 1997; Bugawan et al. 2000). Typing was performed twice independently, and conflicting types were resolved, in most cases, by two independent retyping experiments. TAP1 and TAP2 were genotyped as described elsewhere (Carrington et al. 1993). D6S2971, D6S2749, D6S2874, D6S273, D6S2876, D6S2751, D6S2741, and D6S2739 were typed as described elsewhere (Martin et al. 1998). Genotyping details for the 11 remaining microsatellites can be found as supplemental information on the IDRGWeb site. D6S2972 and D6S265 genotypes were typed twice (IDRG; Martin et al. 1998), and conflicts were resolved by retyping. Alias details for all microsatellites are provided elsewhere (Cullen et al. 2003).

Genotyping Details

Multiplex PCR was performed in six microliter volumes containing 0.1 units of Taq polymerase (Amplitaq Gold, Applied Biosystems), 5 ng genomic DNA, 2.5 pmol of each PCR primer, and 2.5 lmol of dNTP. Thermocycling was at 95° C. for 15 minutes followed by 45 cycles of 95° C. for 20s, 56° C. for 30s, 72° C. for 30s. Unincorporated dNTPs were deactivated using 0.3U of Shrimp Alkaline Phosphatase (Roche) followed by primer extension using 5.4 pmol of each primer extension probe, 50 μmole of the appropriate dNTP/ddNTP combination, and 0.5 units of Thermosequenase (Amersham Pharmacia). Reactions were heated to 94° C. for 2 minutes, followed by 40 cycles of 94° C. for 5 s, 50° C. for 5 s, 72° C. for 5 s. Following addition of a cation exchange resin to remove residual salt from the reactions, seven nanoliters of the purified primer extension reaction was loaded onto a matrix pad (3-hydroxypicoloinic acid) of a SpectroCHIP (Sequenom, San Diego, Calif.). SpectroCHIPs were analyzed using a Bruker Biflex III MALDI-TOF mass spectrometer (SpectroREADER, Sequenom, San Diego, Calif.) and spectra processed using SpectroTYPER (Sequenom).

Eleven of the microsatellites were amplified using the following primers/amplification programs:

| | Forward Primer (5′ to 3′) | Reverse Primer (5′ to 3′) | PCR |
|---|---|---|---|
| Microsatellite | (SEQ ID NOS: 1279-1289) | (SEQ ID NOS: 1290-1300) | program |
| D6S1542 | ACTGGGTGCATCAGGGAG | CTTTACAACCCTTGGCAGC | EPA |
| D6S1560 | CTCCAGTCCCCACTGC | CCCAAGGCCACATAGC | 64ANN |
| D6S1701 | GGTGTCAGAGCAANATTCC | AACAAAGTATCACAAACTGGGAG | RG-MSATS |
| D6S2747 | GGAGACACATTCAAACCATAGG | CAATTGGTGACATACATCAACTTG | MSATTD |
| D6S2896 | AATGGCTGTTAGGAAGAAGC | TCTTCCTTAGCTGCTGCTG | MSATTD |
| D6S2793 | AATAGCCATGAGAAGCTATGTGGGGGAG | CTACCTCCTTGCCAAAGTTGCTGTTTGTG | RG-MSATS |
| D6S2814 | GTGAAATCAGCCTGCTTCTG | GAACACAACCATCTCTGCTC | RG-MSATS |
| D6S2840 | AGATGGCATTTGGAGAGTGCAG | TCCTTACAGCAGAGATATGTGG | RG-MSATS |
| D6S265 | ACGTTCGTACCCATTAACCT | ATCGAGGTAAACAGCAGAAA | RG-MSATS |
| D6S2972 | GAAATGTGAGAATAAAGGAGA | GATAAAGGGGAACTACTACA | EPA |
| D6S258 | GCAAATCAAGAATGTAATTCCC | CTTCCAATCCATAAGCATGG | MSATJH |

The forward primers were fluorescently tagged with 6-FAM, TET or HEX. Amplification was performed in 15 microliter volumes containing 0.8 units Taq polymerase (Roche Applied Science), 25 ng DNA, 200 μM dNTPs, 2.4 pmol each primer, 3.0 mmol dNTPs, and 1×PCR buffer (1.5 mM MgCl2, 10 mM Tris-HCl, 50 mM KCl, pH 8.3, Roche Applied Science). Reactions were run in one of the following MJ Research thermocyclers (PTC-100, PTC-200 or Genomyx CycLR). Samples were then multiplexed and 2-3 μl of multiplex was combined with an equal amount of size standard loading buffer mix (containing formamide, blue dextran and fluorescently labeled size standard Genescan-350 or -500 Tamara), denatured for 3 minutes at 95° C., and electrophoresed on a 5% gel (National Diagnostics), using an ABI model 377 DNA sequencer (Applied Biosystems).

Genotypes for individuals from families 1331, 1332, 1347, 1362, 1413, 1416 and 884 for D6S1542, D6S1560, D6S1701, D6S1666, D6S265, D6S258 were obtained from the CEPH website (http://www.cephb.fr/test/cephdb/). To ensure correspondence in allele sizes with those genotyped for this study, individual 1347-2 was genotyped for these loci.

For RG-MSATS amplification, reactions were heated to 95° C. for 2 minutes followed by 29 cycles of 94° C. for 45 s, 57° C. for 45 s, 72° C. for 1 minute. The final extension was at 72° C. for 7 minutes. MSATJH and 64ANN were the same as RG-MSATS, except annealing was carried out at 55° C. and 64° C. respectively. MSATTD was a touchdown annealing starting at 60° C. and decreasing in each subsequent cycle by 0.3° C. until arriving at 55° C. were annealing was held constant for the remaining 15 cycles. EPA reactions were heated to 95° C. for 2 minutes followed by 4 cycles at 96° C. for 30 s, 57° C. for 90 s and 72° C. for 90 s; followed by 28 cycles at 95° C. for 30 s, 55° C. for 45 s, 72° C. for 1 minute. The final extension was at 72° C. for 30 minutes.

D'Confidence Limits, Definition of Haplotype Blocks, and Structure Comparison

Pairwise D′ values—estimates of the strength of LD (Lewontin 1964)—for SNP markers were assessed and haplotype blocks were defined as per Gabriel et al. (2002). In brief, D′ confidence limits were determined by calculating the probability of the observed data for all possible values of D′, from which an overall probability distribution was determined. For all blocks identified, the outermost marker pair was required to be in strong LD, with an upper confidence limit (CU)>0.98 and a lower confidence limit (CL)>0.7. Blocks defined by only two markers required confidence bounds of (CL)>0.8 and (CU)>0.98 and an intervening distance of =<20 kb; for three consecutive markers, all pairs had to have confidence bounds of CL>0.5 and CU>0.98 and an intervening distance of <30 kb; and for four markers, the fraction of informative pairs in strong LD (CL>0.7 and CU>0.98) was required to be >95%, with an intervening distance of <30 kb. For runs of five or more markers, the fraction of informative pairs in strong LD was required to be >95%, and markers were allowed to span any distance.

SNP genotypes from Gabriel et al. (2002) were used for comparison of haplotype block structure. As the density of coverage was different between these two studies, 20 data sets were derived from the Gabriel et al. (2002) data by randomly removing markers to achieve the same average spacing and spacing distribution. Since there were two existing 100-kb gaps in the SNP coverage described herein, owing to a lack of available SNPs to type near FLOT1 and DQB1, comparison was done by segmenting the MHC into three parts at these gaps.

Phase Inference for Extended-Haplotype-Homozygosity Analysis

Initial SNP, HLA, TAP, and microsatellite chromosomal phasing was done, on the basis of segregation analysis, using the Genehunter program (Kruglyak et al. 1996). The bulk of genotypes—91.6% of SNP genotypes and 95% of HLA, TAP, and microsatellite genotypes—were phased with family information. Apart from initial phasing with family information, HLA, TAP, and microsatellite genotypes were not phased further, and the 5% of genotypes that were indeterminate were considered “ambiguous” in further analyses. Further haplotype inference of SNP genotyping data was performed with a procedure that is based on a probability model for haplotypes proposed elsewhere (Fearnhead and Donnelly 2001). This model can be regarded as a refinement that allows for recombination of the model used in the well-known program, PHASE (Stephens et al. 2001). Both unphased and missing SNP data were inferred in this manner. Since a dense set of markers were used, and most markers are in strong LD with several other markers, the phasing unlikely introduced serious bias into the results.

Extended-Haplotype-Homozygosity Analysis

Extended-haplotype-homozygosity (EHH) analysis was performed, as described elsewhere (Sabeti et al. 2002), for each haplotype block, microsatellite, HLA, and TAP allele, with cM estimations used as distance. Grandparental chromosomes from all families were analyzed. However, some microsatellite types (D6S258, D6S2840, D6S2814, D6S2793, D6S1666, D6S1701, D6S1560, and D6S1542) were not determined for five of these families (1346, 1345, 1420, 1350, and 13292). Rather than infer genotypes, these genotypes were left as “null calls.” As mentioned above, 5% of microsatellite, HLA, and TAP genotypes could not be phased with family information. Since EHH is a cumulative statistic, these heterozygotes and missing data are predicted to result in a conservative estimate of EHH values.

Outlying variants, depicted in FIGS. 3A-3C, were chosen on the basis of two criteria designed to pick alleles with high EHH values for their frequency class. First, as a simple approximation of the distribution, scores were ranked by EHH value times allele frequency. Outliers had values >4.5 SDs above the mean. Second, all variants were sorted by frequency into 5% bins. Outliers had EHH values >=4.79 SDs above the mean for the remaining values in that bin.

Analysis of SNP Haplotypes around HLA-A, HLA-B, HLA-C, and HLA-DRB1

Subsequent to the initial SNP genotyping and analysis of the entire region, additional SNP genotyping was performed near HLA-A, HLA-B, HLA-C, and HLA-DRB1 to assess the correlation between the HLA genotype and local SNP haplotype. Multiblock SNP haplotypes include information from the blocks indicated in FIG. 4, as well as that from any intervening SNPs not in those blocks. “Leave-one-out” cross-validation was performed using the LeaveOneOut program. In brief, a single chromosome is selected from the data set. The remaining samples are used to build a predictor. This predictor is then used to predict the HLA genotype of the sample that has been removed. If the SNP haplotype occurred once, it is not considered in the test. For each locus, prediction was performed with 106 iterations. (See the IDRG Web site for the LeaveOneOut program and genotyping details.)

### Example 2

### Analysis of the MHC Region Based on the Integrated Map

Structure of LD in the HLA Genes, Compared with the Genome at Large

Recent studies have shown that LD extends across long segments of the genome (Daly et al. 2001; Dawson et al. 2002; Gabriel et al. 2002; Phillips et al. 2003). Within such segments, a small number of distinct, common patterns of sequence variation (haplotype alleles) are observed in the general population. Between these segments are short intervals where recombination is apparently most active in creating assortments of these patterns (Daly et al. 2001; Jeffreys et al. 2001; Gabriel et al. 2002). Operationally, it is not necessary to test each variant within an LD segment for association with disease phenotype. Rather, a small subset of variants that identifies all common haplotype alleles within a segment can be used.

In order to compare the LD structure in the MHC with that of the genome as a whole, this MHC data was compared with the data set from Gabriel et al. (2002), as this data set offers a genomewide comparison in which the same CEPH samples were genotyped. The empirical definition of an LD segment or “haplotype block” described in Gabriel et al. (2002) was used, as it provides a common measure for comparison of genomic regions (see “Materials and Methods” section). Because the SNP coverage described herein is less dense than that of Gabriel et al. (2002), subsets of markers were randomly selected from the Gabriel et al. (2002) study to create a data set with a spacing similar to that of the present study and thus appropriate for comparison (see “Materials and Methods” section). Given the SNP coverage used, all haplotype blocks are not detected. At this density, only 25% of the MHC and 14.5% of the Gabriel et al. (2002) data set is found to lie in blocks, compared with 85% when using the full density in the Gabriel et al. (2002) data set.

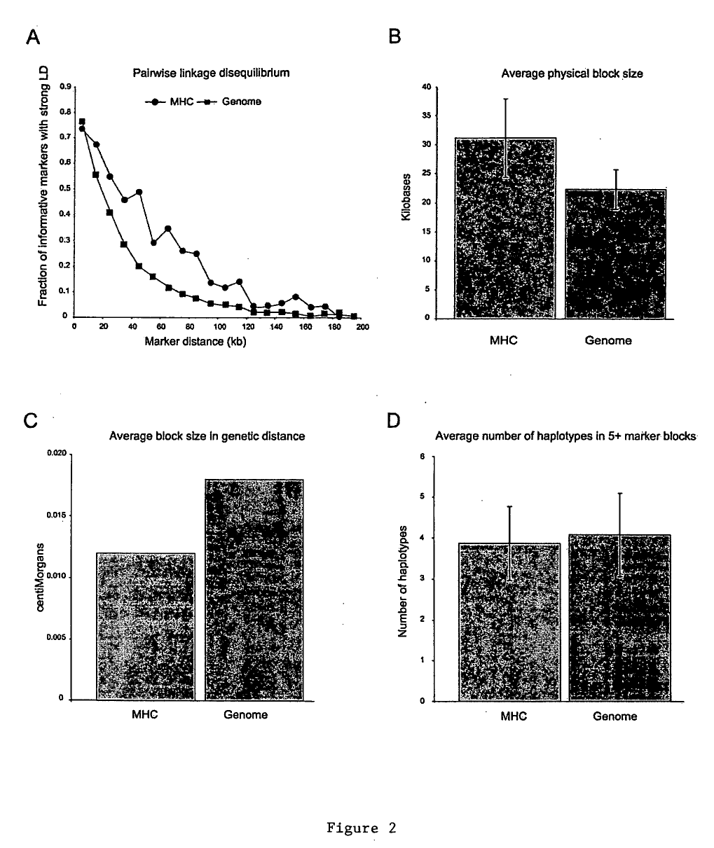

This analysis shows that that LD extends over greater physical distances in the MHC than elsewhere in the genome (FIG. 2A). Seventeen LD segments were identified in the region that meet the criteria of haplotype blocks (Gabriel et al. 2002) (FIGS. 1A-1E). These MHC blocks are longer, on average, in physical distance than those found in the rest of the genome, although this finding does not reach significance, likely because of the small sample size (average length of 31.1 kb vs. 22.3 kb) (FIG. 2B).

Despite being longer in physical distance, haplotype blocks in the MHC are actually shorter, in terms of genetic distance. The average recombination rate in the MHC is 0.49 cM/Mb, versus 0.81 cM/Mb in the genome as a whole (Cullen et al. 2002; Kong et al. 2002). Given this difference in recombination rate, it was found that blocks in the MHC have an average length of 0.012 cM, whereas the average is 0.017 cM for the genomewide control data set (significance not tested) (FIG. 2C). Furthermore, the distribution of recombination across the region correlates well with most of the long blocks (FIG. 1, asterisks) in the region. Six of the seven largest blocks (>=75 kb) lie in areas where recombination rate is well below the genome average of 0.81 cM/Mb. Moreover, five of these blocks lie in regions where the recombination rate is below the MHC regional average of 0.49 cM/Mb. The remaining large block falls into a region where the rate is 0.83 cM/Mb. This leads to a conclusion that the extent of LD in the MHC is longer in physical distance but not in genetic distance than elsewhere in the genome.

Extended-Haplotype Analysis

This work looked for alleles of haplotype blocks, microsatellites, or classical HLA genes that occur on haplotypes that extend across multiple blocks. Such so called “extended haplotypes” are believed to represent a common feature of the MHC (Alper et al. 1992). To analyze the long-range structure of the region, EHH analysis was used, which determines the length of the chromosomal haplotypes that extend from a specific allele at a particular locus (Sabeti et al. 2002). High-frequency, extended haplotypes may result from positive selection or haplotype-specific recombination suppression. Positive selection brings rare alleles to higher frequency in relatively few generations, thus affording fewer opportunities for recombination events to separate an allele from its original chromosomal context. Alternatively, haplotype-specific recombinational suppression may result in high-frequency, extended haplotypes by reducing the number of recombination events a given haplotype will undergo. Since there is a detailed sperm-typing recombination map of the region, this was used to control for positional variation in average recombination rates that would artificially affect the length of haplotypes. Utilizing the integrated haplotype map, the entire MHC was scanned, using each HLA gene, TAP gene, microsatellite, and haplotype block as an independent locus from which to determine EHH values, assessing every allele from a total of 46 loci.

The 50 regions in the Gabriel et al. (2002) data set each span only 250 kb and are, therefore, not long enough to serve as a suitable control data set for this analysis. Thus, the EHH values of haplotype, microsatellite, and gene alleles within the MHC data set were compared with each other and allelic variants that are outliers were identified, on the basis of statistical rank of the EHH value at 0.25 cM, relative to allele frequency (see “Materials and Methods” section) (FIG. 3A). Nine alleles were identified that map onto three different extended haplotypes (FIG. 3B). It is striking that six of these nine variants map to a single multigene haplotype (HLAC*0702-D6S2793*244-DRB1*1501-DQA1*0102-DQB1*0602-D6S2876*11 [hereafter referred to as “DR2”]). Every element in the DR2 haplotype has an EHH value at least 4.8 SDs above the mean EHH for other variants with the same allele frequency. Two of the remaining outlying alleles map to a single haplotype (D6S2840*219-C*0701), and the last outlying allele is DRB1*1101. As noted above, there are at least two possible underlying causes for these extended haplotypes. One possibility is that a variant on the haplotype has experienced recent positive selection. It is interesting that each of the three extended haplotypes has been implicated elsewhere in autoimmune disease (Thorsby 1997; Klein and Sato 2000). The DR2 haplotype is associated with systemic lupus erythematosus (SLE [MIM 152700]) and multiple sclerosis (MS [MIM 126200]) susceptibility, and it is protective for type I diabetes (IDDM [MIM 222100]) (Thorsby 1997; Chataway et al. 1998; Haines et al. 1998; Barcellos et al. 2002). DRB1*1101 is associated with pemphigoid vulgaris, and D6S2840*219-C*0701 is associated with autoimmune diabetes (MIM 275000) and thyroid disease (MIM 140300) (Drouet et al. 1998; Price et al. 1999; Okazaki et al. 2000). Thus, these three haplotypes appear to have functional consequences for the human immune system. Although these haplotypes are associated with autoimmune diseases at present, it is possible that, under certain conditions, these functional differences were (and perhaps still are) beneficial for disease resistance and, therefore, may have undergone positive selection in the past.

The other possibility is that these extended haplotypes are subject to allele-specific recombination suppression. By examining the individual recombination rates used to construct the recombination map, it is observed that, of the 12 individuals examined, the single individual bearing DRB1*1501 showed many fewer recombination events across the MHC than did the others, although this difference did not significantly deviate from the mean. This suggests that allele-specific recombination suppression could be a possibility in this case. Further sperm typing of additional individuals bearing each of these extended haplotypes should resolve whether the underlying cause of this extended haplotype is haplotype-specific recombinational suppression or whether recent positive selection is more likely.

Common Patterns of Sequence Variation in the MHC in Regions Between the Classical HLA Loci

Next, the haplotype block variation in the MHC was compared with the rest of the genome. With the initial coverage, blocks that spanned classical loci were not identified. These blocks have the same number of common patterns of sequence variation (haplotype alleles) as found in other regions of the genome (3.9 vs. 4.1 for blocks with five or more markers) (FIG. 1D). Furthermore, the same percentage of rare haplotype alleles in both data sets (3%) is seen, which indicates that the MHC, aside from the classical loci, does not appear to have an excess of rare haplotype variants detectable at the current marker density. The observation that the diversity of haplotypes outside the classical loci is typical of the rest of the genome is perhaps surprising, given the high level of variation at the classical HLA genes.

Common Variation in Regions Spanning the Classical HLA Loci

The SNP haplotype diversity were separately analyzed in regions spanning the classical HLA genes (but outside the highly variable exons) to understand how this variation is structured. For this purpose, it was necessary to increase the density of SNP coverage by three- to five-fold around the four HLA genes chosen for analysis, HLA-A, HLA-B, HLA-C, and HLA-DRB1. One motivating question in this analysis was whether SNP haplotypes spanning classical HLA loci contained enough information to predict HLA alleles. If so, it might be possible to use high-throughput SNP genotyping as a first-pass surrogate for traditional HLA gene molecular typing (e.g., probe-based typing or direct sequencing) in disease association studies. For one of these classical genes, HLA-A, a single 7-SNP haplotype block spanning the locus was identified. This 7-SNP HLA-A block has only six common variants, and those are predictive of the correct HLA-A allele 66.2% of the time, as shown by cross-validation analysis (LeaveOneOut [see the “Materials and Methods” section]). To capture more of the variation at this locus, the genotype information for a neighboring block was included, and the SNP haplotypes that comprised the combinations of alleles of these two blocks were examined. The success of prediction improved from 66.2% to 82.6% of all HLA-A alleles present.

Using such multiblock haplotypes for all four classical HLA loci studied, multiblock SNP haplotypes can act as surrogate markers for HLA alleles. For example, the HLA-A*0101 allele occurs on the “G” SNP haplotype (comprising the haplotype alleles of two blocks) 92% of the time (FIG. 4A), and the “G” SNP haplotype correlates to HLA-A*010195.6% of the time (FIG. 4B). Cross-validation analysis was used to estimate the success rate of prediction. Even with the current coverage, HLA alleles can be accurately predicted by SNP haplotype 75%/-84% of the time (HLA-A: 82.6%; HLA-B: 79.8%; HLA-C, 84.3%; and HLA-DRB1: 75.0%). Considering only haplotypes bearing common HLA alleles (allele frequency 15%), predictions are accurate at a higher rate (HLA-A: 96.2%, HLA-B: 98.8%; HLA-C, 96.0%; and HLA-DRB1: 82.2%) was found, which suggests that the bulk of the prediction failures reflect an inability to predict low-frequency variants. These data suggest that two elements are needed to improve the predictive power: (1) a larger data set, which would increase the numbers of observations of rare HLA variants, and (2) increased marker density that would provide additional SNP haplotype information, as evidenced by the case of HLA-A above.

Electronic-Database Information

URLs for data presented herein are as follows and are incorporated herein by reference:
Coriell Institute, http://locus.umdnj.edu/ccr/
dbSNP, http://www.ncbi.nlm.nih.gov/SNP/
IDRG, http://www.genome.wi.mit.edu/mpg/idrg/ (for supplementary “Materials and Methods” information and pairwise D′ analysis for 201 reliable, polymorphic SNP assays in 18 multigenerational European CEPH families);
http://www.broad.mit.edu/mpg/idrg/projects/hla.htm.
   Online Mendelian Inheritance in Man (OMIM), http://www.ncbi.nlm.nih.gov/Omim/ (for SLE, MS, IDDM, Hashimoto thyroiditis, Graves disease).

### REFERENCES

Alper C A, Awdeh Z, Yunis E J (1992) Conserved, extended MHC haplotypes. Exp Clin Immunogenet 9:58-71.
Barcellos L F, Oksenberg J R, Green A J, Bucher P, Rimmler J B, Schmidt S, Garcia M E Lincoln R R, Pericak-Vance M A, Haines J L, Hauser S L, Multiple Sclerosis Genetic Group (2002) Genetic basis for clinical expression in multiple sclerosis. Brain 125:150158.
Begovich A B, McClure G R, Suraj V C, Helmuth R C, Fildes N, Bugawan T L, Erlich H A, Klitz W (1992) Polymorphism, recombination, and linkage disequilibrium within the HLA class II region. J Immunol 148:249-258.
Bugawan T L, Klitz W, Blair A, Erlich H A (2000) High-resolution HLA class I typing in the CEPH families: analysis of linkage disequilibrium among HLA loci. Tissue Antigens 56: 392404.
Carrington M, Colonna M, Spies T, Stephens J C, Mann D L (1993) Haplotypic variation of the transporter associated with antigen processing (TAP) genes and their extension of HLA class II region haplotypes. Immunogenetics 37:266-273.
Carrington M, Nelson G W, Martin M P, Kissner T, Vlahov D, Goedert J J, Kaslow R, Buchbinder S, Hoots K, O'Brien S J (1999) HLA and HIV-1: heterozygote advantage and B*35-Cw*04 disadvantage. Science 283:1748-1752.
Carrington M, Stephens J C, Klitz W, Begovich A B, Erlich H A, Mann D (1994) Major histocompatibility complex class II haplotypes and linkage disequilibrium values observed in the CEPH families. Hum Immunol 41:234-240.
Chataway J, Feakes R, Coraddu F, Gray J, Deans J, Fraser M, Robertson N, Broadley S, Jones H, Clayton D, Goodfellow P, Sawcer S, Compston A (1998) The genetics of multiple sclerosis: principles, background and updated results of the United Kingdom systematic genome screen. Brain 121:1869-1887.
Cullen M, Malasky M, Harding A, Carrington M (2003) High-density map of short tandem repeats across the human major histocompatibility complex. Immunogenetics 54(12):900-10.
Cullen M, Perfetto S P, KlitzW, Nelson G, Carrington M (2002) High-resolution patterns of meiotic recombination across the human major histocompatibility complex. Am J Hum Genet 71:759-776.
Daly M J, Rioux J D, Schafffier S F, Hudson T J, Lander E S (2001) High-resolution haplotype structure in the human genome. Nat Genet 29:229-232.
Dawson E, Abecasis G R, Bumpstead S, Chen Y, Hunt S, Beare D M, Pabial J, et al (2002) A first-generation linkage disequilibrium map of human chromosome 22. Nature 418: 544-548.
Drouet M, Delpuget-Bertin N, Vaillant L, Chauchaix S, Boulanger M D, Bonnetblanc J M, Bernard P (1998) HLA-DRB1 and HLA-DQB1 genes in susceptibility and resistance to cicatricial pemphigoid in French Caucasians. Eur J Dermatol 8:330-333.
Fearnhead P, Donnelly P (2001) Estimating recombination rates from population genetic data. Genetics 159:1299-1318.
Gabriel S B, Schafflier S F, Nguyen H, Moore J M, Roy J, Blumenstiel B, Higgins J, DeFelice M, Lochner A, Faggart M, Liu-Cordero S N, Rotimi C, Adeyemo A, Cooper R, Ward R, Lander E S, Daly M J, Altshuler D (2002) The structure of haplotype blocks in the human genome. Science 296:2225-2229.
Haines J L, Terwedow H A, Burgess K, Pericak-Vance M A, Rimmler J B, Martin E R, Oksenberg J R, Lincoln R, Zhang D Y, Banatao D R, Gatto N, Goodkin D E, SL H (1998) Linkage of the MHC to familial multiple sclerosis suggests genetic heterogeneity. The Multiple Sclerosis Genetics Group. Human Molecular Genetics 7:1229-1234.
Jeffreys A J, Kauppi L, Neumann R (2001) Intensely punctuate meiotic recombination in the class II region of the major histocompatibility complex. Nat Genet 29:217-222.
Klein J, Sato A (2000) The HLA system: second of two parts. N Engl J Med 343:782-786.
Kong A, Gudbjartsson D F, Sainz J, Jonsdottir G M, Gudjonsson S A, Richardsson B, Sigurdardottir S, Barnard J, Hallbeck B, Masson G, Shlien A, Palsson S T, Frigge M L, Thorgeirsson T E, Gulcher J R, Stefansson K (2002) A high-resolution recombination map of the human genome. Nat Genet 31:241-247.
Kruglyak L, Daly M J, Reeve-Daly M P, Lander E S (1996) Parametric and nonparametric linkage analysis: a unified multipoint approach. Am J Hum Genet 58:1347-1363.
Lewontin R C (1964) The interaction of selection and linkage. I. General considerations; heterotic models. Genetics 49:49-67.
Martin M P, Harding A, Chadwick R, Kronick M, Cullen M, Lin L, Mignot E, Carrington M (1998) Characterization of 12 microsatellite loci of the human MHC in a panel of reference cell lines. Immunogenetics 47:131-138.
Moonsamy P V, Klitz W, Tilanus M G, Begovich A B (1997) Genetic variability and linkage disequilibrium within the DP region in the CEPH families. Hum Immunol 58:112-121.
Okazaki A, Miyagawa S, Yamashina Y, Kitamura W, Shirai T (2000) Polymorphisms of HLA-DR and -DQ genes in Japanese patients with bullous pemphigoid. J Dermatol 27:149-156.
Phillips M S, Lawrence R, Sachidanandam R, Morris A P, Balding D J, Donaldson M A, Studebaker J F, et al (2003) Chromosome-wide distribution of haplotype blocks and the role of recombination hot spots. Nat Genet 33:382-387.
Price P, Witt C, Allcock R, Sayer D, Garlepp M, Kok C C, French M, Mallal S, Christiansen F (1999) The genetic basis for the association of the 8.1 ancestral haplotype (Al, B8, DR3) with multiple immunopathological diseases. Immunol Rev 167:257-274.
Sabeti P C, Reich D E, Higgins J M, Levine H Z, Richter D J, Schaffier S F, Gabriel S B, Platko J V, Patterson N J, McDonald G J, Ackerman H C, Campbell S J, Altshuler D, Cooper R, Kwiatkowski D,Ward R, Lander E S (2002) Detecting recent positive selection in the human genome from haplotype structure. Nature 419:832-837.
Stephens M, Smith N J, Donnelly P (2001) A new statistical method for haplotype reconstruction from population data. Am J Hum Genet 68:978-989.
Thorsby E (1997) Invited anniversary review: HLA associated diseases. Hum Immunol 53:1-11.

All references cited herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

1. An SNP-haplotype map of a 4-Mb MHC region, said map comprising evenly spaced SNPs, genotyped HLA genes, TAP genes, and microsatellites.

2. The SNP-haplotype map according to claim 1, wherein the SNPs are spaced at approximately every 20 kb of said MHC region.

3. A method of determining the identity of an HLA allele, said method comprising
a) determining the nucleotide present at one or more extra-genic SNP sites corresponding to the HLA allele to be assessed; and
b) identifying said HLA allele based on the nucleotide identity determined in a).

4. The method according to claim 3, wherein the HLA allele is an HLA-A allele.

5. The method according to claim 4, wherein said one ore more SNP sites are selected from the group consisting of: rs2517862, rs1655930, rs1616549, rs376253, rs1961135, rs2517706, rs2517701, rs2517699, rs435766, rs410909, rs2394255, rs1264807, rs2530388, rs356963, rs2286405, rs2240619, rs3129012, and rs259938.

6. The method according to claim 4 comprising determining the nucleotide present at more than one SNP site selected from the group consisting of: rs2517862, rs1655930, rs1616549, rs376253, rs1961135, rs2517706, rs2517701, rs2517699, rs435766, rs410909, rs2394255, rs1264807, rs2530388, rs356963, rs2286405, rs2240619, rs3129012, and rs259938.

7. The method according to claim 3, wherein the HLA allele is an HLA-DRB1 allele.

8. The method according to claim 7, wherein said one ore more SNP sites are selected from the group consisting of: rs742697, rs523627, rs3129960, rs2395163, rs2395165, rs983561, rs2239804, rs2213584, rs2395182, and rs2858860.

9. The method according to claim 8 comprising additionally determining the nucleotide present at one or more SNP sites selected from the group consisting of rs3129907, rs1059544, and rs1987529.

10. The method according to claim 7 comprising determining the nucleotide present at more than one SNP site selected from the group consisting of: rs742697, rs523627, rs3129960, rs2395163, rs2395165, rs983561, rs2239804, rs2213584, rs2395182, rs2858860 rs3129907, rs1059544, and rs1987529.

11. A method of predicting the likelihood of development of an MHC-linked disease or an autoimmune disease in a human, comprising determining the identity of an HLA allele in the human by determining the nucleotide present at one or more extra-genic SNP sites corresponding to the HLA allele to be assessed, wherein if the HLA allele in the human is associated with an MHC-linked disease or an autoimmune disease, the human has a greater likelihood of development of said disease.

12. A method of predicting the likelihood of development of a host-graft response in a human host, comprising determining the identity of an HLA allele of the graft by determining the nucleotide present at one or more extra-genic SNP sites corresponding to the HLA allele to be assessed in the graft, wherein if the HLA allele in the human host is identical to the corresponding HLA allele in the graft, there is a low likelihood of development of a host-graft response in the human host.

13. The method according to claim 12, optionally comprising additionally determining the identity of the corresponding HLA allele of the human host by determining the nucleotide present at one or more extra-genic SNP sites corresponding to the HLA allele to be assessed in the human host.

14. The method according to claim 12, comprising determining the HLA-A allele in the graft by determining the nucleotide present at one or more SNP sites selected from the group consisting of: rs2517862, rs1655930, rs1616549, rs376253, rs1961135, rs2517706, rs2517701, rs2517699, rs435766, rs410909, rs2394255, rs1264807, rs2530388, rs356963, rs2286405, rs2240619, rs3129012, and rs259938.

15. The method according to claim 12, comprising determining the HLA-DRB1 allele in the graft by determining the nucleotide present at one or more SNP sites selected from the group consisting of: rs742697, rs523627, rs3129960, rs2395163, rs2395165, rs983561, rs2239804, rs2213584, rs2395182, rs2858860 rs3129907, rs1059544, and rs1987529.

16. A method of predicting the likelihood of development of a host-graft response in a human host, comprising determining the identity of an HLA allele of the graft by determining the nucleotide present at one or more extra-genic SNP sites corresponding to the HLA allele to be assessed in the graft, wherein if the HLA allele in the human host is different from the corresponding HLA allele in the graft, there is a high likelihood of developing a host-graft response in the human host.

17. The method according to claim 16, optionally comprising additionally determining the identity of the corresponding HLA allele of the human host by determining the nucleotide present at one or more extra-genic SNP sites corresponding to the HLA allele to be assessed in the human host.

18. The method according to claim 16, comprising determining the HLA-A allele in the graft by determining the nucleotide present at one or more SNP sites selected from the group consisting of: rs2517862, rs1655930, rs1616549, rs376253, rs1961135, rs2517706, rs2517701, rs2517699, rs435766, rs410909, rs2394255, rs1264807, rs2530388, rs356963, rs2286405, rs2240619, rs3129012, and rs259938.

19. The method according to claim 16, comprising determining the HLA-DRB1 allele in the graft by determining the nucleotide present at one or more SNP sites selected from the group consisting of: rs742697, rs523627, rs3129960, rs2395163, rs2395165, rs983561, rs2239804, rs2213584, rs2395182, rs2858860 rs3129907, rs1059544, and rs1987529.


SEQUENCE LISTING (1100 sequences)

SEQ ID NO: 1 - DNA, 30 bp, Artificial Sequence
  SNP primer
acgttggatgtgaggaccacaaaagtcagg

SEQ ID NO: 2 - DNA, 30 bp, Artificial Sequence
  SNP primer
acgttggatggggaaaacagcaaggtacag

SEQ ID NO: 3 - DNA, 30 bp, Artificial Sequence
  SNP primer
acgttggatgtgtagccagtcatagctgtc

SEQ ID NO: 4 - DNA, 30 bp, Artificial Sequence
  SNP primer
acgttggatggctatctgcccttttgctac

SEQ ID NO: 5 - DNA, 30 bp, Artificial Sequence
  SNP primer
acgttggatggataaatgggtgttgtttcg

SEQ ID NO: 6 - DNA, 30 bp, Artificial Sequence
  SNP primer
acgttggatgggccccatgatgtataaatg

SEQ ID NO: 7 - DNA, 30 bp, Artificial Sequence
  SNP primer
acgttggatgacaatacctgcagtaccctc

SEQ ID NO: 8 - DNA, 30 bp, Artificial Sequence
  SNP primer
acgttggatgggctcaacagactcggaatg

SEQ ID NO: 9 - DNA, 30 bp, Artificial Sequence
  SNP primer
acgttggatgtccctggaatactgatgagg

SEQ ID NO: 10 - DNA, 30 bp, Artificial Sequence
  SNP primer
acgttggatgtgtagtaatcctagtgctgg

SEQ ID NO: 11 - DNA, 30 bp, Artificial Sequence
  SNP primer
acgttggatgtttggtgccagagagtaagc

SEQ ID NO: 12 - DNA, 30 bp, Artificial Sequence
  SNP primer
acgttggatgagttcacgtggacatccatg

SEQ ID NO: 13 - DNA, 30 bp, Artificial Sequence
  SNP primer
acgttggatggggttatggtgcatacgttc

SEQ ID NO: 14 - DNA, 30 bp, Artificial Sequence
  SNP primer
acgttggatggaaccctccttttcagtgac

SEQ ID NO: 15 - DNA, 30 bp, Artificial Sequence
  SNP primer
acgttggatggtcagaaaagatgggcagac

SEQ ID NO: 16 - DNA, 30 bp, Artificial Sequence
  SNP primer
acgttggatgataggctcctttgcagaagg

SEQ ID NO: 17 - DNA, 30 bp, Artificial Sequence
  SNP primer
acgttggatgggattagaagcatgagccac

SEQ ID NO: 18 - DNA, 30 bp, Artificial Sequence
  SNP primer
acgttggatgctccattctctgtctcaaag

SEQ ID NO: 19 - DNA, 30 bp, Artificial Sequence
  SNP primer
acgttggatgagtctttgagggaaaggagg

SEQ ID NO: 20 - DNA, 30 bp, Artificial Sequence
  SNP primer
acgttggatgattggagtcatgggaacctg

SEQ ID NO: 21 - DNA, 30 bp, Artificial Sequence
  SNP primer
acgttggatgccccacttctcacatgatac

SEQ ID NO: 22 - DNA, 30 bp, Artificial Sequence
  SNP primer
acgttggatgtccatgcctttctgtgtggg

SEQ ID NO: 23 - DNA, 30 bp, Artificial Sequence
  SNP primer
acgttggatgcacacagattcacacacacg

SEQ ID NO: 24 - DNA, 30 bp, Artificial Sequence
  SNP primer
acgttggatgatctccccccttcttcagag

SEQ ID NO: 25 - DNA, 30 bp, Artificial Sequence
  SNP primer
acgttggatgggtgtaaaggaaactgcagg

SEQ ID NO: 26 - DNA, 30 bp, Artificial Sequence
  SNP primer
acgttggatgtaacattcccctttctccac

SEQ ID NO: 27 - DNA, 30 bp, Artificial Sequence
  SNP primer
acgttggatggcacaattctcatccgacac

SEQ ID NO: 28 - DNA, 30 bp, Artificial Sequence
  SNP primer
acgttggatgcccaactctcaacaaggtag

SEQ ID NO: 29 - DNA, 30 bp, Artificial Sequence
  SNP primer
acgttggatgaacacagaaggcagaggttg

SEQ ID NO: 30 - DNA, 30 bp, Artificial Sequence
  SNP primer
acgttggatgtgtggttgctccattcatgc

SEQ ID NO: 31 - DNA, 30 bp, Artificial Sequence
  SNP primer
acgttggatgaaaacaggcagtgcatgagc

SEQ ID NO: 32 - DNA, 30 bp, Artificial Sequence
  SNP primer
acgttggatgattcctctccgtcaggacag

SEQ ID NO: 33 - DNA, 30 bp, Artificial Sequence
  SNP primer
acgttggatgacaaggttctactgaagggc

SEQ ID NO: 34 - DNA, 30 bp, Artificial Sequence
  SNP primer
acgttggatgcccacttggcatggtgaatc

SEQ ID NO: 35 - DNA, 30 bp, Artificial Sequence
  SNP primer
acgttggatgctggacatttcatcaggacc

SEQ ID NO: 36 - DNA, 30 bp, Artificial Sequence
  SNP primer
acgttggatggaggctatggtaccaaactg

SEQ ID NO: 37 - DNA, 30 bp, Artificial Sequence
  SNP primer
acgttggatgctcggactcctttgctcatt

SEQ ID NO: 38 - DNA, 30 bp, Artificial Sequence
  SNP primer
acgttggatgactttcactccactgccttc

SEQ ID NO: 39 - DNA, 30 bp, Artificial Sequence
  SNP primer
acgttggatgcgtctccctttgtaagacag

SEQ ID NO: 40 - DNA, 31 bp, Artificial Sequence
  SNP primer
acgttggatgtcgcggagttgttggtggaag

SEQ ID NO: 41 - DNA, 30 bp, Artificial Sequence
  SNP primer
acgttggatgttttggagctaggattctgg

SEQ ID NO: 42 - DNA, 30 bp, Artificial Sequence
  SNP primer
acgttggatgccctctatgctcactatctc

SEQ ID NO: 43 - DNA, 30 bp, Artificial Sequence
  SNP primer
acgttggatgattttttcaggctccctgtg

SEQ ID NO: 44 - DNA, 30 bp, Artificial Sequence
  SNP primer
acgttggatgagtggggtcacaatgtcttc

SEQ ID NO: 45 - DNA, 30 bp, Artificial Sequence
  SNP primer
acgttggatggatagtgcccattcacactg

SEQ ID NO: 46 - DNA, 30 bp, Artificial Sequence
  SNP primer
acgttggatgagtcattggcctgtttttcg

SEQ ID NO: 47 - DNA, 30 bp, Artificial Sequence
  SNP primer
acgttggatggggaaaaaaaaccgtaagtg

SEQ ID NO: 48 - DNA, 30 bp, Artificial Sequence
  SNP primer
acgttggatgagtcttgtggctgcaagaac

SEQ ID NO: 49 - DNA, 30 bp, Artificial Sequence
  SNP primer
acgttggatgatccaaagcacccaaacctg

SEQ ID NO: 50 - DNA, 30 bp, Artificial Sequence
  SNP primer
acgttggatgttctttgcactccacctctg

SEQ ID NO: 51 - DNA, 30 bp, Artificial Sequence
  SNP primer
acgttggatgtagccaaagaaatcctgagc

SEQ ID NO: 52 - DNA, 30 bp, Artificial Sequence
  SNP primer
acgttggatgacttccacttcccagtagac

SEQ ID NO: 53 - DNA, 30 bp, Artificial Sequence
  SNP primer
acgttggatgcataaaaagcttccaccagc

SEQ ID NO: 54 - DNA, 30 bp, Artificial Sequence
  SNP primer
acgttggatgacacttggcttactttcccc

SEQ ID NO: 55 - DNA, 30 bp, Artificial Sequence
  SNP primer
acgttggatgtggatttttcttccccactc

SEQ ID NO: 56 - DNA, 30 bp, Artificial Sequence
  SNP primer
acgttggatgaactcctgacgtgatctgcc

SEQ ID NO: 57 - DNA, 30 bp, Artificial Sequence
  SNP primer
acgttggatgcgcaaaaaaaagttgcagtc

SEQ ID NO: 58 - DNA, 30 bp, Artificial Sequence
  SNP primer
acgttggatgaccatgattctgaggcctcc

SEQ ID NO: 59 - DNA, 30 bp, Artificial Sequence
  SNP primer
acgttggatgcagtgtgaaattggaccctg

SEQ ID NO: 60 - DNA, 30 bp, Artificial Sequence
  SNP primer
acgttggatgagtcatgaaagatccacccc

SEQ ID NO: 61 - DNA, 30 bp, Artificial Sequence
  SNP primer
acgttggatgttagcaggtatggtggcatg

SEQ ID NO: 62 - DNA, 30 bp, Artificial Sequence
  SNP primer
acgttggatgcatggcctcttatgagaacc

SEQ ID NO: 63 - DNA, 30 bp, Artificial Sequence
  SNP primer
acgttggatgaggtgtgttggaaggtggtg

SEQ ID NO: 64 - DNA, 30 bp, Artificial Sequence
  SNP primer
acgttggatgttgattccccctgctgcttc

SEQ ID NO: 65 - DNA, 30 bp, Artificial Sequence
  SNP primer
acgttggatgctcagaactgctgaaaactg

SEQ ID NO: 66 - DNA, 30 bp, Artificial Sequence
  SNP primer
acgttggatgtctgccttctttgctcaagc

SEQ ID NO: 67 - DNA, 30 bp, Artificial Sequence
  SNP primer
acgttggatggcaagtagcaggaaattcag

SEQ ID NO: 68 - DNA, 30 bp, Artificial Sequence
  SNP primer
acgttggatgcccagtggcaaatcaattac

SEQ ID NO: 69 - DNA, 30 bp, Artificial Sequence
  SNP primer
acgttggatgcctcctagtttctgctttcc

SEQ ID NO: 70 - DNA, 30 bp, Artificial Sequence
  SNP primer
acgttggatgtcaaaggactgcaggaacag

SEQ ID NO: 71 - DNA, 30 bp, Artificial Sequence
  SNP primer
acgttggatgaaatgtcagagtggtgtggg

SEQ ID NO: 72 - DNA, 30 bp, Artificial Sequence
  SNP primer
acgttggatgtgtcgcctcctgcacttcat

SEQ ID NO: 73 - DNA, 30 bp, Artificial Sequence
  SNP primer
acgttggatgccagcagttcattcctgaac

SEQ ID NO: 74 - DNA, 30 bp, Artificial Sequence
  SNP primer
acgttggatgttcttctctctcttcttctc

SEQ ID NO: 75 - DNA, 30 bp, Artificial Sequence
  SNP primer
acgttggatgggaaactatcacaaggacag

SEQ ID NO: 76 - DNA, 30 bp, Artificial Sequence
  SNP primer
acgttggatgtatgattcccctcctccttc

SEQ ID NO: 77 - DNA, 30 bp, Artificial Sequence
  SNP primer
acgttggatgtgccatgatggtaagcttcc

SEQ ID NO: 78 - DNA, 30 bp, Artificial Sequence
  SNP primer
acgttggatgacttccctttcttagccacc

SEQ ID NO: 79 - DNA, 31 bp, Artificial Sequence
  SNP primer
acgttggatgcctggagctttatagtaagtc

SEQ ID NO: 80 - DNA, 30 bp, Artificial Sequence
  SNP primer
acgttggatgaggtccttgattctggactc

SEQ ID NO: 81 - DNA, 30 bp, Artificial Sequence
  SNP primer
acgttggatgagaatcctggactctcaagg

SEQ ID NO: 82 - DNA, 30 bp, Artificial Sequence
  SNP primer
acgttggatggggacaccgcacagattttc

SEQ ID NO: 83 - DNA, 30 bp, Artificial Sequence
  SNP primer
acgttggatgttgagaccatcctggctaac

SEQ ID NO: 84 - DNA, 31 bp, Artificial Sequence
  SNP primer
acgttggatggtgagatcaagagtactattc

SEQ ID NO: 85 - DNA, 30 bp, Artificial Sequence
  SNP primer
acgttggatgggttgtgctaacctgacttc

SEQ ID NO: 86 - DNA, 30 bp, Artificial Sequence
  SNP primer
acgttggatgtaggtatacctgtgccatgg

SEQ ID NO: 87 - DNA, 30 bp, Artificial Sequence
  SNP primer
acgttggatgcagtgccttgatacctgaac

SEQ ID NO: 88 - DNA, 30 bp, Artificial Sequence
  SNP primer
acgttggatgagacatcatgaccattcacc

SEQ ID NO: 89 - DNA, 30 bp, Artificial Sequence
  SNP primer
acgttggatgccaagtggtaagtgagatag

SEQ ID NO: 90 - DNA, 30 bp, Artificial Sequence
  SNP primer
acgttggatgcccaaccctgtaaaatccag

SEQ ID NO: 91 - DNA, 30 bp, Artificial Sequence
  SNP primer
acgttggatgcaagttgccctctctcattg

SEQ ID NO: 92 - DNA, 30 bp, Artificial Sequence
  SNP primer
acgttggatgtcacctttcccacaactctg

SEQ ID NO: 93 - DNA, 30 bp, Artificial Sequence
  SNP primer
acgttggatgatgttgaccaggatggtctc

SEQ ID NO: 94 - DNA, 30 bp, Artificial Sequence
  SNP primer
acgttggatgaattctccctttgggacagg

SEQ ID NO: 95 - DNA, 30 bp, Artificial Sequence
  SNP primer
acgttggatgacgctggcctacatttcaag

SEQ ID NO: 96 - DNA, 30 bp, Artificial Sequence
  SNP primer
acgttggatggaactctgacacaggatcag

SEQ ID NO: 97 - DNA, 30 bp, Artificial Sequence
  SNP primer
acgttggatgcggtgcatctttcatatgag

SEQ ID NO: 98 - DNA, 30 bp, Artificial Sequence
  SNP primer
acgttggatgttgggaggacaaaaaggctg

SEQ ID NO: 99 - DNA, 30 bp, Artificial Sequence
  SNP primer
acgttggatgaggagtttgagaccagcctg

SEQ ID NO: 100 - DNA, 30 bp, Artificial Sequence
  SNP primer
acgttggatggaggagtatggtaggagatg

SEQ ID NO: 101 - DNA, 30 bp, Artificial Sequence
  SNP primer
acgttggatggcacccactgacagtaagag

SEQ ID NO: 102 - DNA, 30 bp, Artificial Sequence
  SNP primer
acgttggatgaaacagaacctgacaccagc

SEQ ID NO: 103 - DNA, 30 bp, Artificial Sequence
  SNP primer
acgttggatgtcctgacccctatcatcctg

SEQ ID NO: 104 - DNA, 30 bp, Artificial Sequence
  SNP primer
acgttggatgcaggcacacagctttttcac

SEQ ID NO: 105 - DNA, 30 bp, Artificial Sequence
  SNP primer
acgttggatgtgatctgcaacctatcccag

SEQ ID NO: 106 - DNA, 30 bp, Artificial Sequence
  SNP primer
acgttggatgatggcgactcacgctccctg

SEQ ID NO: 107 - DNA, 30 bp, Artificial Sequence
  SNP primer
acgttggatgagagttcctctgacccagac

SEQ ID NO: 108 - DNA, 31 bp, Artificial Sequence
  SNP primer
acgttggatgcctctgtactttattttctac

SEQ ID NO: 109 - DNA, 30 bp, Artificial Sequence
  SNP primer
acgttggatgccatgctttttgcaactgcc

SEQ ID NO: 110 - DNA, 30 bp, Artificial Sequence
  SNP primer
acgttggatgaagtgctgggattacagctg

SEQ ID NO: 111 - DNA, 30 bp, Artificial Sequence
  SNP primer
acgttggatgacctgtagtcccagctactc

SEQ ID NO: 112 - DNA, 30 bp, Artificial Sequence
  SNP primer
acgttggatggctctctctaaatgctaggc

SEQ ID NO: 113 - DNA, 30 bp, Artificial Sequence
  SNP primer
acgttggatgacctgagatcgggagcttga

SEQ ID NO: 114 - DNA, 30 bp, Artificial Sequence
  SNP primer
acgttggatgagcttggcttttctccagag

SEQ ID NO: 115 - DNA, 30 bp, Artificial Sequence
  SNP primer
acgttggatgatcccctttgggtgaatctg

SEQ ID NO: 116 - DNA, 30 bp, Artificial Sequence
  SNP primer
acgttggatgcagactgtttgagcctgttg

SEQ ID NO: 117 - DNA, 30 bp, Artificial Sequence
  SNP primer
acgttggatgcccccctcattttgacatcc

SEQ ID NO: 118 - DNA, 30 bp, Artificial Sequence
  SNP primer
acgttggatgagttcccaacccaaatccag

SEQ ID NO: 119 - DNA, 30 bp, Artificial Sequence
  SNP primer
acgttggatggggcaccttgaaaaaagagc

SEQ ID NO: 120 - DNA, 30 bp, Artificial Sequence
  SNP primer
acgttggatgtttctgcaacctctgcctcc

SEQ ID NO: 121 - DNA, 30 bp, Artificial Sequence
  SNP primer
acgttggatgtctttaagtggatgggctcg

SEQ ID NO: 122 - DNA, 30 bp, Artificial Sequence
  SNP primer
acgttggatgaggttgaagcgattctcctg

SEQ ID NO: 123 - DNA, 30 bp, Artificial Sequence
  SNP primer
acgttggatgaagaccacttcagagtccag

SEQ ID NO: 124 - DNA, 31 bp, Artificial Sequence
  SNP primer
acgttggatgctattccaaaacatcactggc

SEQ ID NO: 125 - DNA, 30 bp, Artificial Sequence
  SNP primer
acgttggatgagcctcccacccactcaaag

SEQ ID NO: 126 - DNA, 30 bp, Artificial Sequence
  SNP primer
acgttggatgccatggttgaaggagaagag

SEQ ID NO: 127 - DNA, 30 bp, Artificial Sequence
  SNP primer
acgttggatgcttggtacaagtgaaactgg

SEQ ID NO: 128 - DNA, 30 bp, Artificial Sequence
  SNP primer
acgttggatgtatgacagcacgtttctgcc

SEQ ID NO: 129 - DNA, 30 bp, Artificial Sequence
  SNP primer
acgttggatggcctggcaacatagcaagac

SEQ ID NO: 130 - DNA, 30 bp, Artificial Sequence
  SNP primer
acgttggatgacccctcatctcctaagctc

SEQ ID NO: 131 - DNA, 30 bp, Artificial Sequence
  SNP primer
acgttggatgaggagggaaggaagtataac

SEQ ID NO: 132 - DNA, 30 bp, Artificial Sequence
  SNP primer
acgttggatggctgcataataaattgcccc

SEQ ID NO: 133 - DNA, 30 bp, Artificial Sequence
  SNP primer
acgttggatgggaaagagattcaggcttgg

SEQ ID NO: 134 - DNA, 30 bp, Artificial Sequence
  SNP primer
acgttggatggaaactagggcagagatcag

SEQ ID NO: 135 - DNA, 30 bp, Artificial Sequence
  SNP primer
acgttggatgtgtgactgatggagaccagg

SEQ ID NO: 136 - DNA, 30 bp, Artificial Sequence
  SNP primer
acgttggatgctccaaaagaggtgtgccta

SEQ ID NO: 137 - DNA, 30 bp, Artificial Sequence
  SNP primer
acgttggatgtctaagagccacttctcagc

SEQ ID NO: 138 - DNA, 30 bp, Artificial Sequence
  SNP primer
acgttggatgaaagagcctgcaaaagccag

SEQ ID NO: 139 - DNA, 30 bp, Artificial Sequence
  SNP primer
acgttggatgttgcctagagctgagttgag

SEQ ID NO: 140 - DNA, 30 bp, Artificial Sequence
  SNP primer
acgttggatgaaaggggcagagcatggaag

SEQ ID NO: 141 - DNA, 30 bp, Artificial Sequence
  SNP primer
acgttggatgttggaggttgttgtgggcac

SEQ ID NO: 142 - DNA, 31 bp, Artificial Sequence
  SNP primer
acgttggatgcaagactgcatacaggaatac

SEQ ID NO: 143 - DNA, 30 bp, Artificial Sequence
  SNP primer
acgttggatgatcaccggaaagaccaaagc

SEQ ID NO: 144 - DNA, 30 bp, Artificial Sequence
  SNP primer
acgttggatgaaacttgggccagtgtcaac

SEQ ID NO: 145 - DNA, 30 bp, Artificial Sequence
  SNP primer
acgttggatgaaaccttcccccaaagactg

SEQ ID NO: 146 - DNA, 30 bp, Artificial Sequence
  SNP primer
acgttggatgaagagaaaaatgggcccagc

SEQ ID NO: 147 - DNA, 31 bp, Artificial Sequence
  SNP primer
acgttggatggggttctttgtgttatacttg

SEQ ID NO: 148 - DNA, 30 bp, Artificial Sequence
  SNP primer
acgttggatgcaactgcagactccaaggtg

SEQ ID NO: 149 - DNA, 30 bp, Artificial Sequence
  SNP primer
acgttggatgtgcctcttacttgtgccttg

SEQ ID NO: 150 - DNA, 30 bp, Artificial Sequence
  SNP primer
acgttggatgaacttcttagcttctataat

SEQ ID NO: 151 - DNA, 30 bp, Artificial Sequence
  SNP primer
acgttggatggagaggtcactagcattagc

SEQ ID NO: 152 - DNA, 30 bp, Artificial Sequence
  SNP primer
acgttggatgaagacagaggtgaggaatgc

SEQ ID NO: 153 - DNA, 30 bp, Artificial Sequence
  SNP primer
acgttggatgggccaaattgacttttcacc

SEQ ID NO: 154 - DNA, 30 bp, Artificial Sequence
  SNP primer
acgttggatgccatgttcaacctttggagg

SEQ ID NO: 155 - DNA, 30 bp, Artificial Sequence
  SNP primer
acgttggatgaatgcagtgcgttgtcccag

SEQ ID NO: 156 - DNA, 30 bp, Artificial Sequence
  SNP primer
acgttggatgtttcaccatcttggccaggc

SEQ ID NO: 157 - DNA, 30 bp, Artificial Sequence
  SNP primer
acgttggatgggcacaaaaatggtaaaggg

SEQ ID NO: 158 - DNA, 30 bp, Artificial Sequence
  SNP primer
acgttggatgtctactgatagttcctgccg

SEQ ID NO: 159 - DNA, 30 bp, Artificial Sequence
  SNP primer
acgttggatgatcctacctgaggctgactc

SEQ ID NO: 160 - DNA, 30 bp, Artificial Sequence
  SNP primer
acgttggatgtctgaaggttgaacctgagg

SEQ ID NO: 161 - DNA, 30 bp, Artificial Sequence
  SNP primer
acgttggatgagttcccatgtctggctgtg

SEQ ID NO: 162 - DNA, 30 bp, Artificial Sequence
  SNP primer
acgttggatggttctgaagtggccaaagcc

SEQ ID NO: 163 - DNA, 31 bp, Artificial Sequence
  SNP primer
acgttggatgtgatcgttcggcagctgcaag

SEQ ID NO: 164 - DNA, 31 bp, Artificial Sequence
  SNP primer
acgttggatgatcgttcggcagctgcaagag

SEQ ID NO: 165 - DNA, 31 bp, Artificial Sequence; identical to SEQ ID NO: 164
  SNP primer

SEQ ID NO: 166 - DNA, 30 bp, Artificial Sequence
  SNP primer
acgttggatgaggttggaagaacacacagg

SEQ ID NO: 167 - DNA, 30 bp, Artificial Sequence
  SNP primer
acgttggatgtgaagcctcgggtatctagg

SEQ ID NO: 168 - DNA, 30 bp, Artificial Sequence
  SNP primer
acgttggatgtctctcaatagcctgccctc

SEQ ID NO: 169 - DNA, 30 bp, Artificial Sequence
  SNP primer
acgttggatgcccaaacaggagatcctatc

SEQ ID NO: 170 - DNA, 30 bp, Artificial Sequence
  SNP primer
acgttggatggtcttggagaatgagtgagg

SEQ ID NO: 171 - DNA, 30 bp, Artificial Sequence
  SNP primer
acgttggatgtcttctctgtcaaccacatc

SEQ ID NO: 172 - DNA, 30 bp, Artificial Sequence
  SNP primer
acgttggatgggaggcactaaatattcacg

SEQ ID NO: 173 - DNA, 30 bp, Artificial Sequence
  SNP primer
acgttggatgtttggcctagtttgagtgcc

SEQ ID NO: 174 - DNA, 31 bp, Artificial Sequence
  SNP primer
acgttggatgagaaagaaagaagagagagag

SEQ ID NO: 175 - DNA, 30 bp, Artificial Sequence
  SNP primer
acgttggatgacctctctgctctctgtctc

SEQ ID NO: 176 - DNA, 30 bp, Artificial Sequence
  SNP primer
acgttggatgcaagactcctttcctgtaac

SEQ ID NO: 177 - DNA, 30 bp, Artificial Sequence
  SNP primer
acgttggatgtctgaacccacacagtacac

SEQ ID NO: 178 - DNA, 30 bp, Artificial Sequence
  SNP primer
acgttggatgtttctccatgttggtcaggc

SEQ ID NO: 179 - DNA, 30 bp, Artificial Sequence
  SNP primer
acgttggatgaaaacattgcaggagctgac

SEQ ID NO: 180 - DNA, 30 bp, Artificial Sequence
  SNP primer
acgttggatgtaccatgaatatagctatcg

SEQ ID NO: 181 - DNA, 30 bp, Artificial Sequence
  SNP primer
acgttggatggggtctagagaagtaggttg

SEQ ID NO: 182 - DNA, 30 bp, Artificial Sequence
  SNP primer
acgttggatgtttgcatccctagtcctgag

SEQ ID NO: 183 - DNA, 30 bp, Artificial Sequence
  SNP primer
acgttggatggtctgctaaacacaggtttc

SEQ ID NO: 184 - DNA, 30 bp, Artificial Sequence
  SNP primer
acgttggatgagcaatcacagcaaaggaac

SEQ ID NO: 185 - DNA, 30 bp, Artificial Sequence
  SNP primer
acgttggatgcaaagccacaatgagatacc

SEQ ID NO: 186 - DNA, 30 bp, Artificial Sequence
  SNP primer
acgttggatgtcatgctgaaaggctgtgtg

SEQ ID NO: 187 - DNA, 30 bp, Artificial Sequence
  SNP primer
acgttggatggtggtcccatgaatgctttc

SEQ ID NO: 188 - DNA, 31 bp, Artificial Sequence
  SNP primer
acgttggatgctgaaagtggactgtgaaatg

SEQ ID NO: 189 - DNA, 30 bp, Artificial Sequence
  SNP primer
acgttggatgaaaccctagccagtcttgag

SEQ ID NO: 190 - DNA, 30 bp, Artificial Sequence
  SNP primer
acgttggatgcagtgactttgttgccttgc

SEQ ID NO: 191 - DNA, 30 bp, Artificial Sequence
  SNP primer
acgttggatgattccactttacccagtgtc

SEQ ID NO: 192 - DNA, 30 bp, Artificial Sequence
  SNP primer
acgttggatgcctggaggatgagcaatgac

SEQ ID NO: 193 - DNA, 30 bp, Artificial Sequence
  SNP primer
acgttggatgtgggagcaggagcactgcaa

SEQ ID NO: 194 - DNA, 31 bp, Artificial Sequence
  SNP primer
acgttggatggacttttacgatcatcacttc

SEQ ID NO: 195 - DNA, 30 bp, Artificial Sequence
  SNP primer
acgttggatgctataatcaaagcctgggac

SEQ ID NO: 196 - DNA, 30 bp, Artificial Sequence
  SNP primer
acgttggatgttccctcatgttgttgctgg

SEQ ID NO: 197 - DNA, 30 bp, Artificial Sequence
  SNP primer
acgttggatgccttcagataaggtattggg

SEQ ID NO: 198 - DNA, 30 bp, Artificial Sequence
  SNP primer
acgttggatgtccccaagaggttttcacag

SEQ ID NO: 199 - DNA, 30 bp, Artificial Sequence
  SNP primer
acgttggatgtgcatggctgtaaattaggc

SEQ ID NO: 200 - DNA, 30 bp, Artificial Sequence
  SNP primer
acgttggatgccccttccttgtatagttcc

SEQ ID NO: 201 - DNA, 30 bp, Artificial Sequence
  SNP primer
acgttggatgttgaaccatgaggaggagtc

SEQ ID NO: 202 - DNA, 30 bp, Artificial Sequence
  SNP primer
acgttggatgatgaggtcatgtgagtgtgc

SEQ ID NO: 203 - DNA, 30 bp, Artificial Sequence
  SNP primer
acgttggatgaaagagaatgccctgaatgg

SEQ ID NO: 204 - DNA, 30 bp, Artificial Sequence
  SNP primer
acgttggatgaagacccatttgctgcttcc

SEQ ID NO: 205 - DNA, 30 bp, Artificial Sequence
  SNP primer
acgttggatgagatcacactgacctggcag

SEQ ID NO: 206 - DNA, 31 bp, Artificial Sequence
  SNP primer
acgttggatggaaaacaaatcctagccagtc

SEQ ID NO: 207 - DNA, 30 bp, Artificial Sequence
  SNP primer
acgttggatgaaatatggtgtccctgggac

SEQ ID NO: 208 - DNA, 30 bp, Artificial Sequence
  SNP primer
acgttggatgccatcatccctcacttgaac

SEQ ID NO: 209 - DNA, 30 bp, Artificial Sequence
  SNP primer
acgttggatgaagtatgactcattcacagg

SEQ ID NO: 210 - DNA, 30 bp, Artificial Sequence
  SNP primer
acgttggatgtacttctgatcccctaggac

SEQ ID NO: 211 - DNA, 30 bp, Artificial Sequence
  SNP primer
acgttggatgatgaacaggaccttccaccc

SEQ ID NO: 212 - DNA, 30 bp, Artificial Sequence
  SNP primer
acgttggatgtataactgtcccagctcctg

SEQ ID NO: 213 - DNA, 30 bp, Artificial Sequence
  SNP primer
acgttggatgtactcacccggatcagaatc

SEQ ID NO: 214 - DNA, 30 bp, Artificial Sequence
  SNP primer
acgttggatgttcattggatgagcggtcgg

SEQ ID NO: 215 - DNA, 30 bp, Artificial Sequence
  SNP primer
acgttggatgtgaagtgtggcggtaacggg

SEQ ID NO: 216 - DNA, 30 bp, Artificial Sequence
  SNP primer
acgttggatgtctcttgaactcactagggc

SEQ ID NO: 217 - DNA, 30 bp, Artificial Sequence
  SNP primer
acgttggatgtgaaaccccaatgtctcacc

SEQ ID NO: 218 - DNA, 30 bp, Artificial Sequence
  SNP primer
acgttggatgtcaagtgatcctgctctctc

SEQ ID NO: 219 - DNA, 30 bp, Artificial Sequence
  SNP primer
acgttggatgagttaagagattccctgacc

SEQ ID NO: 220 - DNA, 30 bp, Artificial Sequence
  SNP primer
acgttggatgaggggctcctcacttcccag

SEQ ID NO: 221 - DNA, 29 bp, Artificial Sequence
  SNP primer
acgttggatgaggcagaggggctcctcac

SEQ ID NO: 222 - DNA, 30 bp, Artificial Sequence
  SNP primer
acgttggatgtttaccagatgtctgaaagg

SEQ ID NO: 223 - DNA, 30 bp, Artificial Sequence
  SNP primer
acgttggatgcagaaaagactgggaaagcc

SEQ ID NO: 224 - DNA, 30 bp, Artificial Sequence
  SNP primer
acgttggatggttgatggcaccttcagaag

SEQ ID NO: 225 - DNA, 30 bp, Artificial Sequence
  SNP primer
acgttggatgttctggtgctgccttttgtc

SEQ ID NO: 226 - DNA, 30 bp, Artificial Sequence
  SNP primer
acgttggatgtttctctcttcagttgcccc

SEQ ID NO: 227 - DNA, 30 bp, Artificial Sequence
  SNP primer
acgttggatgaggtggacaaatcacaggtg

SEQ ID NO: 228 - DNA, 30 bp, Artificial Sequence
  SNP primer
acgttggatgtgaggtcaggagttcaagac

SEQ ID NO: 229 - DNA, 30 bp, Artificial Sequence
  SNP primer
acgttggatgttttgactgtccattgcagc

SEQ ID NO: 230 - DNA, 30 bp, Artificial Sequence
  SNP primer
acgttggatgtagagatgactggcttctgg

SEQ ID NO: 231 - DNA, 30 bp, Artificial Sequence
  SNP primer
acgttggatgactttgagaggctgaggttg

SEQ ID NO: 232 - DNA, 30 bp, Artificial Sequence
  SNP primer
acgttggatgtacaccgatttcttcctccc

SEQ ID NO: 233 - DNA, 30 bp, Artificial Sequence
  SNP primer
acgttggatggccttttgcatttgccatgc

SEQ ID NO: 234 - DNA, 30 bp, Artificial Sequence
  SNP primer
acgttggatgatcaaggatacccctcacac

SEQ ID NO: 235 - DNA, 30 bp, Artificial Sequence
  SNP primer
acgttggatggtccctgaacactgtcattc

SEQ ID NO: 236 - DNA, 30 bp, Artificial Sequence
  SNP primer
acgttggatgttgataagacttggccagag

SEQ ID NO: 237 - DNA, 30 bp, Artificial Sequence
  SNP primer
acgttggatggccaacatagtaaaaccccg

SEQ ID NO: 238 - DNA, 30 bp, Artificial Sequence
  SNP primer
acgttggatgattgcaccattgcactcccg

SEQ ID NO: 239 - DNA, 30 bp, Artificial Sequence
  SNP primer
acgttggatgtctctcagcatcccctctag

SEQ ID NO: 240 - DNA, 30 bp, Artificial Sequence
  SNP primer
acgttggatgataactggccagatagggtg

SEQ ID NO: 241 - DNA, 30 bp, Artificial Sequence
  SNP primer
acgttggatgtaagcaggttggagaaacgc

SEQ ID NO: 242 - DNA, 30 bp, Artificial Sequence
  SNP primer
acgttggatgacagtgatggtgggagaagc

SEQ ID NO: 243 - DNA, 30 bp, Artificial Sequence
  SNP primer
acgttggatgccgacaggtctctaaagaag

SEQ ID NO: 244 - DNA, 30 bp, Artificial Sequence
  SNP primer
acgttggatgggttccagcagtctcctaag

SEQ ID NO: 245 - DNA, 30 bp, Artificial Sequence
  SNP primer
acgttggatgtccaggtccaagatcatgtc

SEQ ID NO: 246 - DNA, 30 bp, Artificial Sequence
  SNP primer
acgttggatggagactttgtgcagggttgt

SEQ ID NO: 247 - DNA, 30 bp, Artificial Sequence
  SNP primer
acgttggatgcctcttccaagctaagaacc

SEQ ID NO: 248 - DNA, 30 bp, Artificial Sequence
  SNP primer
acgttggatgagtagctggactacaggtgc

SEQ ID NO: 249 - DNA, 30 bp, Artificial Sequence
  SNP primer
acgttggatggtggagtgacggaagatatg

SEQ ID NO: 250 - DNA, 30 bp, Artificial Sequence
  SNP primer
acgttggatgttaatctccattcagccccc

SEQ ID NO: 251 - DNA, 30 bp, Artificial Sequence
  SNP primer
acgttggatgtcggatctctaggctggatc

SEQ ID NO: 252 - DNA, 30 bp, Artificial Sequence
  SNP primer
acgttggatgaatccttcccctacctcacc

SEQ ID NO: 253 - DNA, 30 bp, Artificial Sequence
  SNP primer
acgttggatgtacatttattccctgagccc

SEQ ID NO: 254 - DNA, 30 bp, Artificial Sequence
  SNP primer
acgttggatgaggagaatcacttgaacccg

SEQ ID NO: 255 - DNA, 30 bp, Artificial Sequence
  SNP primer
acgttggatgcctgtgctgtgttttccagc

SEQ ID NO: 256 - DNA, 30 bp, Artificial Sequence
  SNP primer
acgttggatgaatcgcttgaacctaggagg

SEQ ID NO: 257 - DNA, 30 bp, Artificial Sequence
  SNP primer
acgttggatgtaccatacctgcaactggag

SEQ ID NO: 258 - DNA, 30 bp, Artificial Sequence
  SNP primer
acgttggatgttatcagggcagtcatcacg

SEQ ID NO: 259 - DNA, 30 bp, Artificial Sequence
  SNP primer
acgttggatgatccacctcctcaatggtag

SEQ ID NO: 260 - DNA, 30 bp, Artificial Sequence
  SNP primer
acgttggatgatcttactcgggactgtgag

SEQ ID NO: 261 - DNA, 30 bp, Artificial Sequence
  SNP primer
acgttggatgcattgttctgagaccaaacc

SEQ ID NO: 262 - DNA, 30 bp, Artificial Sequence
  SNP primer
acgttggatggccaagatggaagttaagcc

SEQ ID NO: 263 - DNA, 30 bp, Artificial Sequence
  SNP primer
acgttggatgggaagttaagccttggagac

SEQ ID NO: 264 - DNA, 30 bp, Artificial Sequence
  SNP primer
acgttggatggtgacaccaaggcactctac

SEQ ID NO: 265 - DNA, 30 bp, Artificial Sequence
  SNP primer
acgttggatgtttaatctttggcgggagcg

SEQ ID NO: 266 - DNA, 30 bp, Artificial Sequence
  SNP primer
acgttggatgatgggatctgagccccttgt

SEQ ID NO: 267 - DNA, 30 bp, Artificial Sequence
  SNP primer
acgttggatgcaatatcacgatctgggctc

SEQ ID NO: 268 - DNA, 30 bp, Artificial Sequence
  SNP primer
acgttggatgtgtgagcacactcagcagag

SEQ ID NO: 269 - DNA, 30 bp, Artificial Sequence
  SNP primer
acgttggatgtccagtgagagcagaaatac

SEQ ID NO: 270 - DNA, 30 bp, Artificial Sequence
  SNP primer
acgttggatgcacttcagagaggtttcatg

SEQ ID NO: 271 - DNA, 30 bp, Artificial Sequence
  SNP primer
acgttggatgagtgctcaatgatgcccagg

SEQ ID NO: 272 - DNA, 30 bp, Artificial Sequence
  SNP primer
acgttggatgtgtcccaacagtgcttgtgg

SEQ ID NO: 273 - DNA, 31 bp, Artificial Sequence
  SNP primer
acgttggatgcctgtttattccctgtaatgg

SEQ ID NO: 274 - DNA, 30 bp, Artificial Sequence
  SNP primer
acgttggatgtagctctagaagggcttagg

SEQ ID NO: 275 - DNA, 30 bp, Artificial Sequence
  SNP primer
acgttggatgcctttcttgaaaccaggtgg

SEQ ID NO: 276 - DNA, 30 bp, Artificial Sequence
  SNP primer
acgttggatgactgtacagcatggagctgg

SEQ ID NO: 277 - DNA, 30 bp, Artificial Sequence
  SNP primer
acgttggatgacatgtgtgcaggtgagttg

SEQ ID NO: 278 - DNA, 30 bp, Artificial Sequence
  SNP primer
acgttggatgtctgttggtgcagttgcttg

SEQ ID NO: 279 - DNA, 30 bp, Artificial Sequence
  SNP primer
acgttggatgagctcactgtcttgtgggag

SEQ ID NO: 280 - DNA, 30 bp, Artificial Sequence
  SNP primer
acgttggatgttcccaaaccctcactgtgg

SEQ ID NO: 281 - DNA, 30 bp, Artificial Sequence
  SNP primer
acgttggatgtcaccatctcaggcctggag

SEQ ID NO: 282 - DNA, 30 bp, Artificial Sequence
  SNP primer
acgttggatgcccaggctgatatcaaactc

SEQ ID NO: 283 - DNA, 30 bp, Artificial Sequence
  SNP primer
acgttggatgagagcaaatgcagagactgg

SEQ ID NO: 284 - DNA, 30 bp, Artificial Sequence
  SNP primer
acgttggatggaaggaactcagtttgtcag

SEQ ID NO: 285 - DNA, 30 bp, Artificial Sequence
  SNP primer
acgttggatgaaggcttccatgacctccag

SEQ ID NO: 286 - DNA, 30 bp, Artificial Sequence
  SNP primer
acgttggatgacagtcctcaccggtgaagc

SEQ ID NO: 287 - DNA, 30 bp, Artificial Sequence
  SNP primer
acgttggatgtctcttacccaaggctacac

SEQ ID NO: 288 - DNA, 30 bp, Artificial Sequence
  SNP primer
acgttggatgtccaaacccttccttctgag

SEQ ID NO: 289 - DNA, 30 bp, Artificial Sequence
  SNP primer
acgttggatgggagatgacccaacatcctc

SEQ ID NO: 290 - DNA, 30 bp, Artificial Sequence
  SNP primer
acgttggatgtctcctttacagcttggtgc

SEQ ID NO: 291 - DNA, 30 bp, Artificial Sequence
  SNP primer
acgttggatggcacgtaagcagtgcaaggc

SEQ ID NO: 292 - DNA, 30 bp, Artificial Sequence
  SNP primer
acgttggatggggaacagaggtttatggtc

SEQ ID NO: 293 - DNA, 30 bp, Artificial Sequence
  SNP primer
acgttggatgaaaccctcttccttgtctcc

SEQ ID NO: 294 - DNA, 30 bp, Artificial Sequence
  SNP primer
acgttggatgcactgatcttgacaacacac

SEQ ID NO: 295 - DNA, 30 bp, Artificial Sequence
  SNP primer
acgttggatgtctggccttatccctaacag

SEQ ID NO: 296 - DNA, 30 bp, Artificial Sequence
  SNP primer
acgttggatggaaacatggagtcatgaggc

SEQ ID NO: 297 - DNA, 30 bp, Artificial Sequence
  SNP primer
acgttggatgactgcagtttgtctgctacg

SEQ ID NO: 298 - DNA, 30 bp, Artificial Sequence
  SNP primer
acgttggatgaggaatgagctaggatggag

SEQ ID NO: 299 - DNA, 30 bp, Artificial Sequence
  SNP primer
acgttggatgctctcgtagagctttcattc

SEQ ID NO: 300 - DNA, 30 bp, Artificial Sequence
  SNP primer
acgttggatgaaatagagacggcctccagg

SEQ ID NO: 301 - DNA, 30 bp, Artificial Sequence
  SNP primer
acgttggatgaaactgctcatcaccacacc

SEQ ID NO: 302 - DNA, 30 bp, Artificial Sequence
  SNP primer
acgttggatgcttcctttccaatcttttgg

SEQ ID NO: 303 - DNA, 30 bp, Artificial Sequence
  SNP primer
acgttggatgtgggactatgtgaaaagacc

SEQ ID NO: 304 - DNA, 30 bp, Artificial Sequence
  SNP primer
acgttggatgagagaagcaggagtaaggtg

SEQ ID NO: 305 - DNA, 30 bp, Artificial Sequence
  SNP primer
acgttggatgagtcaggaaacaacagatgc

SEQ ID NO: 306 - DNA, 30 bp, Artificial Sequence
  SNP primer
acgttggatggcctttagaaaatggtccac

SEQ ID NO: 307 - DNA, 30 bp, Artificial Sequence
  SNP primer
acgttggatgaaagaattgggacttgcctc

SEQ ID NO: 308 - DNA, 30 bp, Artificial Sequence
  SNP primer
acgttggatgtgtcctaacttcctgggtac

SEQ ID NO: 309 - DNA, 30 bp, Artificial Sequence
  SNP primer
acgttggatgatcactcaccactcacatgg

SEQ ID NO: 310 - DNA, 30 bp, Artificial Sequence
  SNP primer
acgttggatgagggaaaactttccctttgg

SEQ ID NO: 311 - DNA, 30 bp, Artificial Sequence
  SNP primer
acgttggatgtgacctgctgataaactttc

SEQ ID NO: 312 - DNA, 30 bp, Artificial Sequence
  SNP primer
acgttggatgaccaccacctaagtttccag

SEQ ID NO: 313 - DNA, 30 bp, Artificial Sequence
  SNP primer
acgttggatgaccaccaaagtttccagaac

SEQ ID NO: 314 - DNA, 30 bp, Artificial Sequence
  SNP primer
acgttggatgagaagtgacagggaagctac

SEQ ID NO: 315 - DNA, 30 bp, Artificial Sequence
  SNP primer
acgttggatgactgatggtagggaaaggtg

SEQ ID NO: 316 - DNA, 30 bp, Artificial Sequence
  SNP primer
acgttggatgatcatgccactgcattccag

SEQ ID NO: 317 - DNA, 30 bp, Artificial Sequence
  SNP primer
acgttggatgcttggataggtgatcacttc

SEQ ID NO: 318 - DNA, 30 bp, Artificial Sequence
  SNP primer
acgttggatgctctcttttaccagtgtgag

SEQ ID NO: 319 - DNA, 30 bp, Artificial Sequence
  SNP primer
acgttggatgtatccacagagacaatgtcc

SEQ ID NO: 320 - DNA, 30 bp, Artificial Sequence
  SNP primer
acgttggatgctcaccatcttttggaactg

SEQ ID NO: 321 - DNA, 30 bp, Artificial Sequence
  SNP primer
acgttggatgtacatggctagcacgaaagg

SEQ ID NO: 322 - DNA, 30 bp, Artificial Sequence
  SNP primer
acgttggatgggataagagcaaaaagttag

SEQ ID NO: 323 - DNA, 30 bp, Artificial Sequence
  SNP primer
acgttggatggagagcaatttccaaacctg

SEQ ID NO: 324 - DNA, 31 bp, Artificial Sequence
  SNP primer
acgttggatggttataaggtaagttgaggtc

SEQ ID NO: 325 - DNA, 30 bp, Artificial Sequence
  SNP primer
acgttggatgcctgccacaacataaaaggc

SEQ ID NO: 326 - DNA, 30 bp, Artificial Sequence
  SNP primer
acgttggatgttagtggtactggtgtgtcc

SEQ ID NO: 327 - DNA, 30 bp, Artificial Sequence
  SNP primer
acgttggatgttggtgattgacacagtcac

SEQ ID NO: 328 - DNA, 30 bp, Artificial Sequence
  SNP primer
acgttggatggtactgagtacaccatctag

SEQ ID NO: 329 - DNA, 30 bp, Artificial Sequence
  SNP primer
acgttggatgccctattaatagggtggacc

SEQ ID NO: 330 - DNA, 30 bp, Artificial Sequence
  SNP primer
acgttggatgtccttgcctgcttccttttc

SEQ ID NO: 331 - DNA, 30 bp, Artificial Sequence
  SNP primer
acgttggatggcttaggtctgatcaatctg

SEQ ID NO: 332 - DNA, 30 bp, Artificial Sequence
  SNP primer
acgttggatgaaaacgatgcccctatcagc

SEQ ID NO: 333 - DNA, 31 bp, Artificial Sequence
  SNP primer
acgttggatggggaaagaaaatgtctatggc

SEQ ID NO: 334 - DNA, 30 bp, Artificial Sequence
  SNP primer
acgttggatgttgcacagatgcaaagattc

SEQ ID NO: 335 - DNA, 30 bp, Artificial Sequence
  SNP primer
acgttggatgtttaaaatgtttgtgccatc

SEQ ID NO: 336 - DNA, 30 bp, Artificial Sequence
  SNP primer
acgttggatgatctattccagccaggctag

SEQ ID NO: 337 - DNA, 30 bp, Artificial Sequence
  SNP primer
acgttggatgtttgcagaggatagcaggag

SEQ ID NO: 338 - DNA, 30 bp, Artificial Sequence
  SNP primer
acgttggatgctaataacacactgtgaaac

SEQ ID NO: 339 - DNA, 30 bp, Artificial Sequence
  SNP primer
acgttggatgaggccagtttggatctgaag

SEQ ID NO: 340 - DNA, 30 bp, Artificial Sequence
  SNP primer
acgttggatgtcagtctgccctcgtcaatg

SEQ ID NO: 341 - DNA, 30 bp, Artificial Sequence
  SNP primer
acgttggatgacctgacaggaagcaaaggg

SEQ ID NO: 342 - DNA, 30 bp, Artificial Sequence
  SNP primer
acgttggatgtaggaacaacaggacatggg

SEQ ID NO: 343 - DNA, 31 bp, Artificial Sequence
  SNP primer
acgttggatggcacctcgtatttttatcaag

SEQ ID NO: 344 - DNA, 30 bp, Artificial Sequence
  SNP primer
acgttggatgtacgaggtgctatggtgcag

SEQ ID NO: 345 - DNA, 29 bp, Artificial Sequence
  SNP primer
acgttggatgtattgggaagacatccggg

SEQ ID NO: 346 - DNA, 30 bp, Artificial Sequence
  SNP primer
acgttggatggctgagtcacctttggaaag

SEQ ID NO: 347 - DNA, 30 bp, Artificial Sequence
  SNP primer
acgttggatgtaaaattgggcatgggaaac

SEQ ID NO: 348 - DNA, 30 bp, Artificial Sequence
  SNP primer
acgttggatgtttccctccccacaatctac

SEQ ID NO: 349 - DNA, 30 bp, Artificial Sequence
  SNP primer
acgttggatgatcggcagcttggaaactac

SEQ ID NO: 350 - DNA, 30 bp, Artificial Sequence
  SNP primer
acgttggatgcagcttccatgtggtgtttg

SEQ ID NO: 351 - DNA, 31 bp, Artificial Sequence
  SNP primer
acgttggatgcagttcctatgaaggatgatc

SEQ ID NO: 352 - DNA, 30 bp, Artificial Sequence
  SNP primer
acgttggatgcagtatcccacagagaagtc

SEQ ID NO: 353 - DNA, 30 bp, Artificial Sequence
  SNP primer
acgttggatgacccctttctaatatctctc

SEQ ID NO: 354 - DNA, 30 bp, Artificial Sequence; identical to SEQ ID NO: 353
  SNP primer

SEQ ID NO: 355 - DNA, 30 bp, Artificial Sequence
  SNP primer
acgttggatggctcttcaacttatgatggg

SEQ ID NO: 356 - DNA, 30 bp, Artificial Sequence
  SNP primer
acgttggatgaaactacagacacacctgac

SEQ ID NO: 357 - DNA, 30 bp, Artificial Sequence
  SNP primer
acgttggatgtcatattggccactccgaag

SEQ ID NO: 358 - DNA, 30 bp, Artificial Sequence
  SNP primer
acgttggatgtattccagcagaccagcttc

SEQ ID NO: 359 - DNA, 30 bp, Artificial Sequence
  SNP primer
acgttggatgtacatctctcaggcttgctc

SEQ ID NO: 360 - DNA, 30 bp, Artificial Sequence; identical to SEQ ID NO: 359
  SNP primer

SEQ ID NO: 361 - DNA, 30 bp, Artificial Sequence
  SNP primer
acgttggatgatctgcaacatcagcagagg

SEQ ID NO: 362 - DNA, 30 bp, Artificial Sequence
  SNP primer
acgttggatgtgttacttcttcccacactc

SEQ ID NO: 363 - DNA, 30 bp, Artificial Sequence
  SNP primer
acgttggatgctgaagctttgggataccag

SEQ ID NO: 364 - DNA, 30 bp, Artificial Sequence
  SNP primer
acgttggatgagtgtggatatgcctcttcg

SEQ ID NO: 365 - DNA, 30 bp, Artificial Sequence
  SNP primer
acgttggatgggtaactatgtgtgtcttgc

SEQ ID NO: 366 - DNA, 30 bp, Artificial Sequence
  SNP primer
acgttggatgcatggaggtgatggtgtttc

SEQ ID NO: 367 - DNA, 30 bp, Artificial Sequence
  SNP primer
acgttggatgtctgagatccataccttggg

SEQ ID NO: 368 - DNA, 30 bp, Artificial Sequence
  SNP primer
acgttggatgaaccccaaggtatggatctc

SEQ ID NO: 369 - DNA, 30 bp, Artificial Sequence
  SNP primer
acgttggatgaatgggtaaggccagtcttc

SEQ ID NO: 370 - DNA, 30 bp, Artificial Sequence
  SNP primer
acgttggatgggccttacccattctgttag

SEQ ID NO: 371 - DNA, 31 bp, Artificial Sequence
  SNP primer
acgttggatggacattaagatgagaggaagg

SEQ ID NO: 372 - DNA, 30 bp, Artificial Sequence
  SNP primer
acgttggatgtgcataagcatttcactgag

SEQ ID NO: 373 - DNA, 30 bp, Artificial Sequence
  SNP primer
acgttggatgataatgtgaggaggctagtc

SEQ ID NO: 374 - DNA, 30 bp, Artificial Sequence
  SNP primer
acgttggatgctctagccttctttctgtcc

SEQ ID NO: 375 - DNA, 30 bp, Artificial Sequence
  SNP primer
acgttggatgccaggatccaaacagtgatc

SEQ ID NO: 376 - DNA, 30 bp, Artificial Sequence
  SNP primer
acgttggatgaccaggcctttgattttcag

SEQ ID NO: 377 - DNA, 30 bp, Artificial Sequence
  SNP primer
acgttggatgagcaggcaagtctcacattc

SEQ ID NO: 378 - DNA, 30 bp, Artificial Sequence
  SNP primer
acgttggatgtcagtgcagtcagctgcctc

SEQ ID NO: 379 - DNA, 30 bp, Artificial Sequence
  SNP primer
acgttggatggcaaatctgtcctccaacac

SEQ ID NO: 380 - DNA, 30 bp, Artificial Sequence
  SNP primer
acgttggatgtctgtctcagcccactttgc

SEQ ID NO: 381 - DNA, 30 bp, Artificial Sequence
  SNP primer
acgttggatgccagtttttcagaggatgcc

SEQ ID NO: 382 - DNA, 30 bp, Artificial Sequence
  SNP primer
acgttggatgttccccatacagcaattccc

SEQ ID NO: 383 - DNA, 30 bp, Artificial Sequence
  SNP primer
acgttggatgtatattcccccaccccatag

SEQ ID NO: 384 - DNA, 30 bp, Artificial Sequence
  SNP primer
acgttggatggcaactggttcaaacctttc

SEQ ID NO: 385 - DNA, 30 bp, Artificial Sequence
  SNP primer
acgttggatggcacttagtgcaattctgag

SEQ ID NO: 386 - DNA, 30 bp, Artificial Sequence
  SNP primer
acgttggatgaggcctataaggaatgagtg

SEQ ID NO: 387 - DNA, 30 bp, Artificial Sequence
  SNP primer
acgttggatggcacatagcaatatggctac

SEQ ID NO: 388 - DNA, 30 bp, Artificial Sequence
  SNP primer
acgttggatgttccaaagtaagtgaggcac

SEQ ID NO: 389 - DNA, 30 bp, Artificial Sequence
  SNP primer
acgttggatgtggtttgggaagtgggagtg

SEQ ID NO: 390 - DNA, 30 bp, Artificial Sequence
  SNP primer
acgttggatgggcaaggatgatgtgtctgc

SEQ ID NO: 391 - DNA, 30 bp, Artificial Sequence
  SNP primer
acgttggatgagacacctgcaagcctgcag

SEQ ID NO: 392 - DNA, 31 bp, Artificial Sequence
  SNP primer
acgttggatgttctgcccaggagactgtctg

SEQ ID NO: 393 - DNA, 30 bp, Artificial Sequence
  SNP primer
acgttggatgctgctgacacgagtgggaac

SEQ ID NO: 394 - DNA, 30 bp, Artificial Sequence
  SNP primer
acgttggatgcagatgatgaggtaggatgc

SEQ ID NO: 395 - DNA, 30 bp, Artificial Sequence
  SNP primer
acgttggatggagagagtctattcccttag

SEQ ID NO: 396 - DNA, 30 bp, Artificial Sequence
  SNP primer
acgttggatgcctcacctttctgattcctg

SEQ ID NO: 397 - DNA, 30 bp, Artificial Sequence
  SNP primer
acgttggatgatgttagccaggatggtctc

SEQ ID NO: 398 - DNA, 30 bp, Artificial Sequence
  SNP primer
acgttggatgggttcatagttctccctgag

SEQ ID NO: 399 - DNA, 30 bp, Artificial Sequence
  SNP primer
acgttggatggacagtagcacatgtgagtc

SEQ ID NO: 400 - DNA, 30 bp, Artificial Sequence
  SNP primer
acgttggatgctgcagacctcactctatgg

SEQ ID NO: 401 - DNA, 30 bp, Artificial Sequence
  SNP primer
acgttggatgccaatctctgctcaagtgtg

SEQ ID NO: 402 - DNA, 30 bp, Artificial Sequence
  SNP primer
acgttggatgtacctcattaggcagttgtg

SEQ ID NO: 403 - DNA, 30 bp, Artificial Sequence
  SNP primer
acgttggatgtagagacctccaggctatag

SEQ ID NO: 404 - DNA, 30 bp, Artificial Sequence
  SNP primer
acgttggatggctgcagacatatctaggag

SEQ ID NO: 405 - DNA, 30 bp, Artificial Sequence
  SNP primer
acgttggatgaaagcgtgtgggtgttctag

SEQ ID NO: 406 - DNA, 30 bp, Artificial Sequence
  SNP primer
acgttggatgtcttctggtttggtgagtgc

SEQ ID NO: 407 - DNA, 30 bp, Artificial Sequence
  SNP primer
acgttggatgatattctcaggccactgcac

SEQ ID NO: 408 - DNA, 30 bp, Artificial Sequence
  SNP primer
acgttggatggggttgtaccttgaaaagac

SEQ ID NO: 409 - DNA, 30 bp, Artificial Sequence
  SNP primer
acgttggatgagtactgtccctagtcactg

SEQ ID NO: 410 - DNA, 30 bp, Artificial Sequence
  SNP primer
acgttggatgttcatagagcatgaggagcc

SEQ ID NO: 411 - DNA, 30 bp, Artificial Sequence
  SNP primer
acgttggatgaatgaagatgacaggcagag

SEQ ID NO: 412 - DNA, 31 bp, Artificial Sequence
  SNP primer
acgttggatgctgttcatctctattgacttg

SEQ ID NO: 413 - DNA, 30 bp, Artificial Sequence
  SNP primer
acgttggatgagaaccaaaccttcacttgg

SEQ ID NO: 414 - DNA, 30 bp, Artificial Sequence
  SNP primer
acgttggatgcttcagggcaaaagacaatg

SEQ ID NO: 415 - DNA, 30 bp, Artificial Sequence
  SNP primer
acgttggatgtgacttccttttctcccagg

SEQ ID NO: 416 - DNA, 30 bp, Artificial Sequence
  SNP primer
acgttggatgaggaatgttctctggagctg

SEQ ID NO: 417 - DNA, 31 bp, Artificial Sequence
  SNP primer
acgttggatgaaagcagaaggccctgctgag

SEQ ID NO: 418 - DNA, 30 bp, Artificial Sequence
  SNP primer
acgttggatgtgggcactgtcttcatcatc

SEQ ID NO: 419 - DNA, 30 bp, Artificial Sequence
  SNP primer
acgttggatgagctttcctctccccttctc

SEQ ID NO: 420 - DNA, 31 bp, Artificial Sequence
  SNP primer
acgttggatggacaacaaaaaaaatacttct

SEQ ID NO: 421 - DNA, 30 bp, Artificial Sequence
  SNP primer
acgttggatgggatctgcagagccatcttc

SEQ ID NO: 422 - DNA, 30 bp, Artificial Sequence
  SNP primer
acgttggatgcctcacttaatttgccctac

SEQ ID NO: 423 - DNA, 30 bp, Artificial Sequence
  SNP primer
acgttggatggaagccttcaatgttacagg

SEQ ID NO: 424 - DNA, 30 bp, Artificial Sequence
  SNP primer
acgttggatggtgacactaataacccaagg

SEQ ID NO: 425 - DNA, 30 bp, Artificial Sequence
  SNP primer
acgttggatgagaggacacagtcatctcag

SEQ ID NO: 426 - DNA, 30 bp, Artificial Sequence
  SNP primer
acgttggatgtattccactcccaactcctg

SEQ ID NO: 427 - DNA, 30 bp, Artificial Sequence
  SNP primer
acgttggatggagtgttaaaggtagaagcc

SEQ ID NO: 428 - DNA, 30 bp, Artificial Sequence
  SNP primer
acgttggatgaactgtcgacgtcacacgac

SEQ ID NO: 429 - DNA, 30 bp, Artificial Sequence
  SNP primer
acgttggatgatactagtaggatctcaggc

SEQ ID NO: 430 - DNA, 30 bp, Artificial Sequence
  SNP primer
acgttggatgctcggttctaaccaagtagg

SEQ ID NO: 431 - DNA, 30 bp, Artificial Sequence
  SNP primer
acgttggatgtttcctcaataggtgtagac

SEQ ID NO: 432 - DNA, 30 bp, Artificial Sequence
  SNP primer
acgttggatggaattcacaggcttttagcc

SEQ ID NO: 433 - DNA, 30 bp, Artificial Sequence
  SNP primer
acgttggatgaatgtcatatgcctcctccc

SEQ ID NO: 434 - DNA, 30 bp, Artificial Sequence
  SNP primer
acgttggatgacctgttgggaaatgtaggc

SEQ ID NO: 435 - DNA, 30 bp, Artificial Sequence
  SNP primer
acgttggatgctggcctgagttttgataag

SEQ ID NO: 436 - DNA, 30 bp, Artificial Sequence
  SNP primer
acgttggatgaggacacatgggagatctag

SEQ ID NO: 437 - DNA, 30 bp, Artificial Sequence
  SNP primer
acgttggatgtccaaacagaggatgctcag

SEQ ID NO: 438 - DNA, 30 bp, Artificial Sequence
  SNP primer
acgttggatgtatgcactaacccatcagcg

SEQ ID NO: 439 - DNA, 29 bp, Artificial Sequence
  SNP primer
acgttggatgtgaggcaggaggtcagcac

SEQ ID NO: 440 - DNA, 31 bp, Artificial Sequence
  SNP primer
acgttggatggacttgtttttcatggcgtag

SEQ ID NO: 441 - DNA, 30 bp, Artificial Sequence
  SNP primer
acgttggatgatcactgcctttggtcttgc

SEQ ID NO: 442 - DNA, 30 bp, Artificial Sequence
  SNP primer
acgttggatgatgtttgatagcagactggg

SEQ ID NO: 443 - DNA, 30 bp, Artificial Sequence
  SNP primer
acgttggatgggcagtgtgaactaagagtg

SEQ ID NO: 444 - DNA, 30 bp, Artificial Sequence
  SNP primer
acgttggatggtcagcctctgtataaggac

SEQ ID NO: 445 - DNA, 30 bp, Artificial Sequence
  SNP primer
acgttggatgtttcccacctccacagtttg

SEQ ID NO: 446 - DNA, 30 bp, Artificial Sequence
  SNP primer
acgttggatgtccagtcaaagagtgaaccc

SEQ ID NO: 447 - DNA, 30 bp, Artificial Sequence
  SNP primer
acgttggatgctcctctttacattcccacc

SEQ ID NO: 448 - DNA, 30 bp, Artificial Sequence
  SNP primer
acgttggatgttactgatggtgctgctcac

SEQ ID NO: 449 - DNA, 30 bp, Artificial Sequence
  SNP primer
acgttggatgcacagttccctaacgagaag

SEQ ID NO: 450 - DNA, 30 bp, Artificial Sequence
  SNP primer
acgttggatgtgtgtagatctctagcgagg

SEQ ID NO: 451 - DNA, 30 bp, Artificial Sequence
  SNP primer
acgttggatgagagactgagttcagtgtgg

SEQ ID NO: 452 - DNA, 30 bp, Artificial Sequence
  SNP primer
acgttggatgcaatccattggtgtaacagg

SEQ ID NO: 453 - DNA, 30 bp, Artificial Sequence
  SNP primer
acgttggatgtccactccacagatgatgag

SEQ ID NO: 454 - DNA, 30 bp, Artificial Sequence
  SNP primer
acgttggatgacctcagttttgcatcctgc

SEQ ID NO: 455 - DNA, 30 bp, Artificial Sequence
  SNP primer
acgttggatgttttgtcccccatccctttc

SEQ ID NO: 456 - DNA, 30 bp, Artificial Sequence
  SNP primer
acgttggatgctcagtgattggcacaagtg

SEQ ID NO: 457 - DNA, 30 bp, Artificial Sequence
  SNP primer
acgttggatgggtaccatcagaatactgtc

SEQ ID NO: 458 - DNA, 30 bp, Artificial Sequence
  SNP primer
acgttggatgctaagggaaggagaactctc

SEQ ID NO: 459 - DNA, 30 bp, Artificial Sequence
  SNP primer
acgttggatgtagactgggctatgtagcac

SEQ ID NO: 460 - DNA, 30 bp, Artificial Sequence
  SNP primer
acgttggatgatcccacccccattcttatc

SEQ ID NO: 461 - DNA, 30 bp, Artificial Sequence
  SNP primer
acgttggatgccaagccagtcaacattttc

SEQ ID NO: 462 - DNA, 30 bp, Artificial Sequence
  SNP primer
acgttggatgggtttaggagatgagttggg

SEQ ID NO: 463 - DNA, 30 bp, Artificial Sequence
  SNP primer
acgttggatgccttcttctgtgtctccatc

SEQ ID NO: 464 - DNA, 30 bp, Artificial Sequence
  SNP primer
acgttggatgactgtgtatgcgtgacactc

SEQ ID NO: 465 - DNA, 30 bp, Artificial Sequence
  SNP primer
acgttggatgggtggagagatgtgatttcc

SEQ ID NO: 466 - DNA, 30 bp, Artificial Sequence
  SNP primer
acgttggatgatccaagcactttgggaagc

SEQ ID NO: 467 - DNA, 30 bp, Artificial Sequence
  SNP primer
acgttggatgcctggcttgctttcttcttg

SEQ ID NO: 468 - DNA, 30 bp, Artificial Sequence
  SNP primer
acgttggatgtgagggaacgcatagcgcag

SEQ ID NO: 469 - DNA, 30 bp, Artificial Sequence
  SNP primer
acgttggatgaacacagaaagaccctcatc

SEQ ID NO: 470 - DNA, 30 bp, Artificial Sequence
  SNP primer
acgttggatgtcaccatgagtttcaccacc

SEQ ID NO: 471 - DNA, 31 bp, Artificial Sequence
  SNP primer
acgttggatgaggccactgctcctctgatac

SEQ ID NO: 472 - DNA, 30 bp, Artificial Sequence
  SNP primer
acgttggatggcagcttatgcaagagtgac

SEQ ID NO: 473 - DNA, 30 bp, Artificial Sequence
  SNP primer
acgttggatggccaaggacggcttggaata

SEQ ID NO: 474 - DNA, 30 bp, Artificial Sequence
  SNP primer
acgttggatgcctttatctctgtggacttg

SEQ ID NO: 475 - DNA, 30 bp, Artificial Sequence
  SNP primer
acgttggatgagtggctcatgcctgtaatc

SEQ ID NO: 476 - DNA, 30 bp, Artificial Sequence
  SNP primer
acgttggatgaccatgtggagcaaaaaggc

SEQ ID NO: 477 - DNA, 30 bp, Artificial Sequence
  SNP primer
acgttggatgaggccctcaaaatcacaaac

SEQ ID NO: 478 - DNA, 30 bp, Artificial Sequence
  SNP primer
acgttggatgcacgtgtttatctgctgacc

SEQ ID NO: 479 - DNA, 30 bp, Artificial Sequence
  SNP primer
acgttggatgatgtctggaactggaccctg

SEQ ID NO: 480 - DNA, 30 bp, Artificial Sequence
  SNP primer
acgttggatgttttgtcgaccctgcacttg

SEQ ID NO: 481 - DNA, 30 bp, Artificial Sequence
  SNP primer
acgttggatgaggagaatggcatgaacccc

SEQ ID NO: 482 - DNA, 30 bp, Artificial Sequence
  SNP primer
acgttggatgccaacctttgatatcctggg

SEQ ID NO: 483 - DNA, 30 bp, Artificial Sequence
  SNP primer
acgttggatgccagacagcatgaacagaag

SEQ ID NO: 484 - DNA, 30 bp, Artificial Sequence
  SNP primer
acgttggatgccgaagtgttcggattatag

SEQ ID NO: 485 - DNA, 30 bp, Artificial Sequence
  SNP primer
acgttggatgagtagaagaaggaccacctg

SEQ ID NO: 486 - DNA, 30 bp, Artificial Sequence
  SNP primer
acgttggatgtcaccctttctctttcctcc

SEQ ID NO: 487 - DNA, 30 bp, Artificial Sequence
  SNP primer
acgttggatgaactcactttcctccctagg

SEQ ID NO: 488 - DNA, 30 bp, Artificial Sequence
  SNP primer
acgttggatgagcatcattggcaggtgagg

SEQ ID NO: 489 - DNA, 30 bp, Artificial Sequence
  SNP primer
acgttggatgagaactttccctctagcctg

SEQ ID NO: 490 - DNA, 30 bp, Artificial Sequence
  SNP primer
acgttggatgtctctctttccctgtagctc

SEQ ID NO: 491 - DNA, 30 bp, Artificial Sequence
  SNP primer
acgttggatgttcagcctctggtgtagctg

SEQ ID NO: 492 - DNA, 30 bp, Artificial Sequence
  SNP primer
acgttggatgatagtgctcactggctgaag

SEQ ID NO: 493 - DNA, 30 bp, Artificial Sequence
  SNP primer
acgttggatgatgatggcagcagccactgc

SEQ ID NO: 494 - DNA, 30 bp, Artificial Sequence
  SNP primer
acgttggatgacctctgcagacttagactg

SEQ ID NO: 495 - DNA, 30 bp, Artificial Sequence
  SNP primer
acgttggatgaaacccacacctgcagtgag

SEQ ID NO: 496 - DNA, 30 bp, Artificial Sequence
  SNP primer
acgttggatgtcctttcctctgggttagcc

SEQ ID NO: 497 - DNA, 30 bp, Artificial Sequence
  SNP primer
acgttggatgtttgagatggagtctcgctc

SEQ ID NO: 498 - DNA, 30 bp, Artificial Sequence
  SNP primer
acgttggatgaagcagctgggaaagaaacg

SEQ ID NO: 499 - DNA, 30 bp, Artificial Sequence
  SNP primer
acgttggatgagggcaaatgaaatctgtcc

SEQ ID NO: 500 - DNA, 30 bp, Artificial Sequence
  SNP primer
acgttggatgcccttgccttatctttcttg

SEQ ID NO: 501 - DNA, 30 bp, Artificial Sequence
  SNP primer
acgttggatgagaggaggaagagctgaaag

SEQ ID NO: 502 - DNA, 30 bp, Artificial Sequence
  SNP primer
acgttggatgggctcaccaaccttctcatg

SEQ ID NO: 503 - DNA, 30 bp, Artificial Sequence
  SNP primer
acgttggatgtttcagagacaccagacatg

SEQ ID NO: 504 - DNA, 30 bp, Artificial Sequence
  SNP primer
acgttggatgagaaccctccagatgaactc

SEQ ID NO: 505 - DNA, 30 bp, Artificial Sequence
  SNP primer
acgttggatgcttcctcgggttcctatatc

SEQ ID NO: 506 - DNA, 30 bp, Artificial Sequence
  SNP primer
acgttggatgtctaagacatgagtgctggg

SEQ ID NO: 507 - DNA, 30 bp, Artificial Sequence
  SNP primer
acgttggatgccaagagctctcaagaaggg

SEQ ID NO: 508 - DNA, 30 bp, Artificial Sequence
  SNP primer
acgttggatgtggcctaaattcccggtgag

SEQ ID NO: 509 - DNA, 30 bp, Artificial Sequence
  SNP primer
acgttggatgtctaaattcaggaccctggc

SEQ ID NO: 510 - DNA, 30 bp, Artificial Sequence
  SNP primer
acgttggatgtttggcctcattgttggctg

SEQ ID NO: 511 - DNA, 30 bp, Artificial Sequence
  SNP primer
acgttggatgttcgactcttcccggactcc

SEQ ID NO: 512 - DNA, 30 bp, Artificial Sequence
  SNP primer
acgttggatggctcagcttaaatgtctccc

SEQ ID NO: 513 - DNA, 30 bp, Artificial Sequence
  SNP primer
acgttggatgaacccacctttcttgttggc

SEQ ID NO: 514 - DNA, 30 bp, Artificial Sequence
  SNP primer
acgttggatgggactggatgaggtttttta

SEQ ID NO: 515 - DNA, 30 bp, Artificial Sequence
  SNP primer
acgttggatgtcctccactttttgttggcc

SEQ ID NO: 516 - DNA, 30 bp, Artificial Sequence
  SNP primer
acgttggatgagtcacttgaatgggctcag

SEQ ID NO: 517 - DNA, 30 bp, Artificial Sequence
  SNP primer
acgttggatgtatagctgtggactgggctg

SEQ ID NO: 518 - DNA, 30 bp, Artificial Sequence
  SNP primer
acgttggatggccatgcacaggaaaatcag

SEQ ID NO: 519 - DNA, 30 bp, Artificial Sequence
  SNP primer
acgttggatgtcacgtggtcctttcatcac

SEQ ID NO: 520 - DNA, 30 bp, Artificial Sequence
  SNP primer
acgttggatgaagcaggctttttgcctctc

SEQ ID NO: 521 - DNA, 30 bp, Artificial Sequence
  SNP primer
acgttggatggctgtaggaaacacgtgttc

SEQ ID NO: 522 - DNA, 30 bp, Artificial Sequence
  SNP primer
acgttggatgctttacactttgggccagtg

SEQ ID NO: 523 - DNA, 30 bp, Artificial Sequence
  SNP primer
acgttggatgaatgtcatcaggaagccctg

SEQ ID NO: 524 - DNA, 30 bp, Artificial Sequence
  SNP primer
acgttggatgtctccgatgagcagcattag

SEQ ID NO: 525 - DNA, 30 bp, Artificial Sequence
  SNP primer
acgttggatgccttccatccctccaaattc

SEQ ID NO: 526 - DNA, 30 bp, Artificial Sequence
  SNP primer
acgttggatgtagaatccggcgtgtatgtg

SEQ ID NO: 527 - DNA, 30 bp, Artificial Sequence
  SNP primer
acgttggatgaagtcgacgcccattctgac

SEQ ID NO: 528 - DNA, 30 bp, Artificial Sequence
  SNP primer
acgttggatgagcacttcgttagacactgc

SEQ ID NO: 529 - DNA, 30 bp, Artificial Sequence
  SNP primer
acgttggatgtctctcctcctctttgtccc

SEQ ID NO: 530 - DNA, 30 bp, Artificial Sequence
  SNP primer
acgttggatgagactacgcatcctcttctc

SEQ ID NO: 531 - DNA, 30 bp, Artificial Sequence
  SNP primer
acgttggatgcctgtgcccttatgattctg

SEQ ID NO: 532 - DNA, 30 bp, Artificial Sequence
  SNP primer
acgttggatgaagcatggttctgtgccctg

SEQ ID NO: 533 - DNA, 30 bp, Artificial Sequence
  SNP primer
acgttggatgatgctgagagctcacccttg

SEQ ID NO: 534 - DNA, 30 bp, Artificial Sequence
  SNP primer
acgttggatgactttgaaactgaactgaag

SEQ ID NO: 535 - DNA, 30 bp, Artificial Sequence
  SNP primer
acgttggatgaactggattgggaaagggag

SEQ ID NO: 536 - DNA, 30 bp, Artificial Sequence
  SNP primer
acgttggatgttagttacactgctgctggc

SEQ ID NO: 537 - DNA, 30 bp, Artificial Sequence
  SNP primer
acgttggatgccctgcattgactgtcttac

SEQ ID NO: 538 - DNA, 30 bp, Artificial Sequence
  SNP primer
acgttggatgaatagctgtgtgaccttggg

SEQ ID NO: 539 - DNA, 30 bp, Artificial Sequence
  SNP primer
acgttggatgtagtggctgtttctctcctg

SEQ ID NO: 540 - DNA, 30 bp, Artificial Sequence
  SNP primer
acgttggatgcccatcaattgacccagttc

SEQ ID NO: 541 - DNA, 30 bp, Artificial Sequence
  SNP primer
acgttggatgtgttctttctccctttgcac

SEQ ID NO: 542 - DNA, 30 bp, Artificial Sequence
  SNP primer
acgttggatgacttctcagctgcatcgatg

SEQ ID NO: 543 - DNA, 30 bp, Artificial Sequence
  SNP primer
acgttggatgtggctacatttgacaccctc

SEQ ID NO: 544 - DNA, 30 bp, Artificial Sequence
  SNP primer
acgttggatggcaaaacacggaaaaagttc

SEQ ID NO: 545 - DNA, 30 bp, Artificial Sequence
  SNP primer
acgttggatgacaggtacactgcaaaagtg

SEQ ID NO: 546 - DNA, 30 bp, Artificial Sequence
  SNP primer
acgttggatgaaaacttccctcatcccagc

SEQ ID NO: 547 - DNA, 30 bp, Artificial Sequence
  SNP primer
acgttggatgacgtgagcatatgagggcat

SEQ ID NO: 548 - DNA, 30 bp, Artificial Sequence
  SNP primer
acgttggatgaaagcagagaacaaggcctg

SEQ ID NO: 549 - DNA, 30 bp, Artificial Sequence
  SNP primer
acgttggatgatgggtccaattttccaccc

SEQ ID NO: 550 - DNA, 30 bp, Artificial Sequence
  SNP primer
acgttggatgttctgtctcatgcactcagg

SEQ ID NO: 551 - DNA, 30 bp, Artificial Sequence
  SNP primer
acgttggatgtttgtgctgaagtgtgcagg

SEQ ID NO: 552 - DNA, 30 bp, Artificial Sequence
  SNP primer
acgttggatgtcactccaggactcaggttc

SEQ ID NO: 553 - DNA, 30 bp, Artificial Sequence
  SNP primer
acgttggatgggctgatactctgggttatc

SEQ ID NO: 554 - DNA, 30 bp, Artificial Sequence
  SNP primer
acgttggatgtgctcctcaattccacatgc

SEQ ID NO: 555 - DNA, 30 bp, Artificial Sequence
  SNP primer
acgttggatgaagaaccttgggacacgatg

SEQ ID NO: 556 - DNA, 30 bp, Artificial Sequence
  SNP primer
acgttggatgggcacacaaggtgcattttg

SEQ ID NO: 557 - DNA, 30 bp, Artificial Sequence
  SNP primer
acgttggatgcttgtatctgactgattttc

SEQ ID NO: 558 - DNA, 30 bp, Artificial Sequence
  SNP primer
acgttggatgaaaattcctggtgcccaagg

SEQ ID NO: 559 - DNA, 30 bp, Artificial Sequence
  SNP primer
acgttggatgaccctaggtaagaggatgtg

SEQ ID NO: 560 - DNA, 30 bp, Artificial Sequence
  SNP primer
acgttggatggcctctgtcttctctttctg

SEQ ID NO: 561 - DNA, 30 bp, Artificial Sequence
  SNP primer
acgttggatgtgaggaataggggtcagcag

SEQ ID NO: 562 - DNA, 30 bp, Artificial Sequence
  SNP primer
acgttggatgaagtcagcctgtcccacaac

SEQ ID NO: 563 - DNA, 30 bp, Artificial Sequence
  SNP primer
acgttggatggtctctttttcctggaggtg

SEQ ID NO: 564 - DNA, 30 bp, Artificial Sequence
  SNP primer
acgttggatgatggagacagtcctttccag

SEQ ID NO: 565 - DNA, 31 bp, Artificial Sequence
  SNP primer
acgttggatggagatatatcttaccctaacc

SEQ ID NO: 566 - DNA, 30 bp, Artificial Sequence
  SNP primer
acgttggatgtaacctctgtctccttccag

SEQ ID NO: 567 - DNA, 30 bp, Artificial Sequence
  SNP primer
acgttggatgtcagcctcgttattccttcc

SEQ ID NO: 568 - DNA, 30 bp, Artificial Sequence
  SNP primer
acgttggatgaaagtcttgctcttgtcccc

SEQ ID NO: 569 - DNA, 30 bp, Artificial Sequence
  SNP primer
acgttggatgcagacaaatggcagttagcc

SEQ ID NO: 570 - DNA, 30 bp, Artificial Sequence
  SNP primer
acgttggatgtcacctcaaagttgcaagcg

SEQ ID NO: 571 - DNA, 30 bp, Artificial Sequence
  SNP primer
acgttggatgatctcctgtagatctcccgg

SEQ ID NO: 572 - DNA, 30 bp, Artificial Sequence
  SNP primer
acgttggatgaccatgggctttatgtggtc

SEQ ID NO: 573 - DNA, 30 bp, Artificial Sequence
  SNP primer
acgttggatgaaggtcttaggagagggctg

SEQ ID NO: 574 - DNA, 30 bp, Artificial Sequence
  SNP primer
acgttggatgatctcagcatgtgaggcttc

SEQ ID NO: 575 - DNA, 30 bp, Artificial Sequence
  SNP primer
acgttggatgctgtggattctgggatagag

SEQ ID NO: 576 - DNA, 30 bp, Artificial Sequence
  SNP primer
acgttggatgcccacaggaaaggagaaaag

SEQ ID NO: 577 - DNA, 30 bp, Artificial Sequence
  SNP primer
acgttggatgaaactttcgtgctgcaggtg

SEQ ID NO: 578 - DNA, 30 bp, Artificial Sequence
  SNP primer
acgttggatgactgctgaagagtgacaagc

SEQ ID NO: 579 - DNA, 30 bp, Artificial Sequence
  SNP primer
acgttggatgtaaacttccacagggcctcc

SEQ ID NO: 580 - DNA, 30 bp, Artificial Sequence
  SNP primer
acgttggatgctacctcacttctgcatttc

SEQ ID NO: 581 - DNA, 30 bp, Artificial Sequence
  SNP primer
acgttggatgaaaagagccaagggcaacac

SEQ ID NO: 582 - DNA, 30 bp, Artificial Sequence
  SNP primer
acgttggatgtcagtttctcaacccaaccc

SEQ ID NO: 583 - DNA, 30 bp, Artificial Sequence
  SNP primer
acgttggatgtattgtttgagcctgggagg

SEQ ID NO: 584 - DNA, 30 bp, Artificial Sequence
  SNP primer
acgttggatgaatgtccagagctgatgagg

SEQ ID NO: 585 - DNA, 30 bp, Artificial Sequence
  SNP primer
acgttggatgtgacatctacaaacagtttc

SEQ ID NO: 586 - DNA, 30 bp, Artificial Sequence
  SNP primer
acgttggatgggcagttgtaaatatttttc

SEQ ID NO: 587 - DNA, 30 bp, Artificial Sequence
  SNP primer
acgttggatggggaagaatctgcttccaag

SEQ ID NO: 588 - DNA, 30 bp, Artificial Sequence
  SNP primer
acgttggatgcaaaagacacagctaaagcc

SEQ ID NO: 589 - DNA, 30 bp, Artificial Sequence
  SNP primer
acgttggatgtggatgtgtggtagttcctg

SEQ ID NO: 590 - DNA, 30 bp, Artificial Sequence
  SNP primer
acgttggatgcctgacaaaaatgtctctag

SEQ ID NO: 591 - DNA, 30 bp, Artificial Sequence
  SNP primer
acgttggatgtgtacaagagtgcctacctg

SEQ ID NO: 592 - DNA, 30 bp, Artificial Sequence
  SNP primer
acgttggatgtcatcactgccatcacaagc

SEQ ID NO: 593 - DNA, 30 bp, Artificial Sequence
  SNP primer
acgttggatgagccctggtagtttttgtgg

SEQ ID NO: 594 - DNA, 30 bp, Artificial Sequence
  SNP primer
acgttggatgtaagatgttgccacagttcc

SEQ ID NO: 595 - DNA, 30 bp, Artificial Sequence
  SNP primer
acgttggatgaaaaaataggctgggcacgg

SEQ ID NO: 596 - DNA, 30 bp, Artificial Sequence
  SNP primer
acgttggatgggaattgttgggtgatatgg

SEQ ID NO: 597 - DNA, 30 bp, Artificial Sequence
  SNP primer
acgttggatgctgccgataaagacataccc

SEQ ID NO: 598 - DNA, 30 bp, Artificial Sequence
  SNP primer
acgttggatgtccctaaagggatcaatggc

SEQ ID NO: 599 - DNA, 30 bp, Artificial Sequence
  SNP primer
acgttggatgcccacccaaatttcgtgttc

SEQ ID NO: 600 - DNA, 30 bp, Artificial Sequence
  SNP primer
acgttggatgaactcctggctcaaatgatc

SEQ ID NO: 601 - DNA, 30 bp, Artificial Sequence
  SNP primer
acgttggatggggcaacaagagtgaaactg

SEQ ID NO: 602 - DNA, 30 bp, Artificial Sequence
  SNP primer
acgttggatgcccatgcatgcaattacctc

SEQ ID NO: 603 - DNA, 30 bp, Artificial Sequence
  SNP primer
acgttggatgtcgtgtcagtggaagctggg

SEQ ID NO: 604 - DNA, 30 bp, Artificial Sequence
  SNP primer
acgttggatgagactcgttgctctcttttc

SEQ ID NO: 605 - DNA, 30 bp, Artificial Sequence
  SNP primer
acgttggatgattagctgagtctggtggtg

SEQ ID NO: 606 - DNA, 30 bp, Artificial Sequence
  SNP primer
acgttggatgcctctggaagataaaatggg

SEQ ID NO: 607 - DNA, 30 bp, Artificial Sequence
  SNP primer
acgttggatgaataacccctggctcagaac

SEQ ID NO: 608 - DNA, 30 bp, Artificial Sequence
  SNP primer
acgttggatgctcacagaagagaggatgag

SEQ ID NO: 609 - DNA, 30 bp, Artificial Sequence
  SNP primer
acgttggatggctgcaaatacatgtgtggg

SEQ ID NO: 610 - DNA, 30 bp, Artificial Sequence
  SNP primer
acgttggatgtgagtgggattgaaaagctg

SEQ ID NO: 611 - DNA, 30 bp, Artificial Sequence
  SNP primer
acgttggatgtccaacagacgcttttctgg

SEQ ID NO: 612 - DNA, 30 bp, Artificial Sequence
  SNP primer
acgttggatgtgggctgagttcctcacttg

SEQ ID NO: 613 - DNA, 31 bp, Artificial Sequence
  SNP primer
acgttggatgcgttaatgaatctaggagctg

SEQ ID NO: 614 - DNA, 30 bp, Artificial Sequence
  SNP primer
acgttggatgctcattgttcagtttccacc

SEQ ID NO: 615 - DNA, 30 bp, Artificial Sequence
  SNP primer
acgttggatggaactatgatcctgaccctg

SEQ ID NO: 616 - DNA, 30 bp, Artificial Sequence
  SNP primer
acgttggatgtctgcaggctttatgggaag

SEQ ID NO: 617 - DNA, 30 bp, Artificial Sequence
  SNP primer
acgttggatgcactgggagagatgagtatg

SEQ ID NO: 618 - DNA, 30 bp, Artificial Sequence
  SNP primer
acgttggatggaaggactttgaatatccac

SEQ ID NO: 619 - DNA, 30 bp, Artificial Sequence
  SNP primer
acgttggatgtttcgcgctggggagcctct

SEQ ID NO: 620 - DNA, 30 bp, Artificial Sequence
  SNP primer
acgttggatgatttgtccccaatccatcgg

SEQ ID NO: 621 - DNA, 30 bp, Artificial Sequence
  SNP primer
acgttggatgcctcgctctgattgtagtag

SEQ ID NO: 622 - DNA, 30 bp, Artificial Sequence
  SNP primer
acgttggatgccaccacgcccagaaaattt

SEQ ID NO: 623 - DNA, 30 bp, Artificial Sequence
  SNP primer
acgttggatgctgtgtcacttggtaagtag

SEQ ID NO: 624 - DNA, 30 bp, Artificial Sequence
  SNP primer
acgttggatggtgggaaggattccacaaag

SEQ ID NO: 625 - DNA, 30 bp, Artificial Sequence
  SNP primer
acgttggatgggaagggatagcattaggag

SEQ ID NO: 626 - DNA, 30 bp, Artificial Sequence
  SNP primer
acgttggatgacagcctggatggcttatag

SEQ ID NO: 627 - DNA, 30 bp, Artificial Sequence
  SNP primer
acgttggatgaggcattcaaattggagaag

SEQ ID NO: 628 - DNA, 30 bp, Artificial Sequence
  SNP primer
acgttggatgtctcccagaacttatcacac

SEQ ID NO: 629 - DNA, 30 bp, Artificial Sequence
  SNP primer
acgttggatgttcttagccacagtcagctg

SEQ ID NO: 630 - DNA, 30 bp, Artificial Sequence
  SNP primer
acgttggatgtttcacctcttttcctcggg

SEQ ID NO: 631 - DNA, 30 bp, Artificial Sequence
  SNP primer
acgttggatgacaaacaggaaggaggcaag

SEQ ID NO: 632 - DNA, 30 bp, Artificial Sequence
  SNP primer
acgttggatgtaatcccagcactttgggag

SEQ ID NO: 633 - DNA, 30 bp, Artificial Sequence
  SNP primer
acgttggatggggattatgctggaggtagc

SEQ ID NO: 634 - DNA, 30 bp, Artificial Sequence
  SNP primer
acgttggatgttttcctggagaggaagagg

SEQ ID NO: 635 - DNA, 30 bp, Artificial Sequence
  SNP primer
acgttggatgccaccccatgaggaataatg

SEQ ID NO: 636 - DNA, 30 bp, Artificial Sequence
  SNP primer
acgttggatgccatggaacactcctgaaag

SEQ ID NO: 637 - DNA, 30 bp, Artificial Sequence
  SNP primer
acgttggatgtgtgccggaaaaaacacagg

SEQ ID NO: 638 - DNA, 30 bp, Artificial Sequence
  SNP primer
acgttggatgtgagaagctgggactacagg

SEQ ID NO: 639 - DNA, 30 bp, Artificial Sequence
  SNP primer
acgttggatgaagccagtctttgcagtagc

SEQ ID NO: 640 - DNA, 30 bp, Artificial Sequence
  SNP primer
acgttggatgtagggagaaagatcgaaggg

SEQ ID NO: 641 - DNA, 30 bp, Artificial Sequence
  SNP primer
acgttggatgtcccaaatgttcccacaagc

SEQ ID NO: 642 - DNA, 31 bp, Artificial Sequence
  SNP primer
acgttggatgtatgctctgggagccctcaac

SEQ ID NO: 643 - DNA, 30 bp, Artificial Sequence
  SNP primer
acgttggatgtcagttggagaaacccacac

SEQ ID NO: 644 - DNA, 30 bp, Artificial Sequence
  SNP primer
acgttggatgtatggctaacttgtcctggc

SEQ ID NO: 645 - DNA, 30 bp, Artificial Sequence
  SNP primer
acgttggatgtagaggtcccaaggtagctg

SEQ ID NO: 646 - DNA, 31 bp, Artificial Sequence
  SNP primer
acgttggatgttgcagcagagctgggacaag

SEQ ID NO: 647 - DNA, 30 bp, Artificial Sequence
  SNP primer
acgttggatgtgaggagacaggcagggtag

SEQ ID NO: 648 - DNA, 30 bp, Artificial Sequence
  SNP primer
acgttggatgttccatccctagtttctgcc

SEQ ID NO: 649 - DNA, 30 bp, Artificial Sequence
  SNP primer
acgttggatgcagacagcaggtatggtaag

SEQ ID NO: 650 - DNA, 30 bp, Artificial Sequence
  SNP primer
acgttggatgtctcggctcactacaatctc

SEQ ID NO: 651 - DNA, 30 bp, Artificial Sequence
  SNP primer
acgttggatgagcagtcagcatcaaagctc

SEQ ID NO: 652 - DNA, 30 bp, Artificial Sequence
  SNP primer
acgttggatgttacaggcatgcaccaccac

SEQ ID NO: 653 - DNA, 30 bp, Artificial Sequence
  SNP primer
acgttggatgtccatggagtaggtacaagg

SEQ ID NO: 654 - DNA, 30 bp, Artificial Sequence
  SNP primer
acgttggatgagacttgtcattccaggtcc

SEQ ID NO: 655 - DNA, 30 bp, Artificial Sequence
  SNP primer
acgttggatgaagttgaaaacctccagccc

SEQ ID NO: 656 - DNA, 30 bp, Artificial Sequence
  SNP primer
acgttggatgtcatggcccacatgactgtg

SEQ ID NO: 657 - DNA, 30 bp, Artificial Sequence
  SNP primer
acgttggatggatgcacaacatcaagaagc

SEQ ID NO: 658 - DNA, 30 bp, Artificial Sequence
  SNP primer
acgttggatgaaatatggctctgttccgcc

SEQ ID NO: 659 - DNA, 30 bp, Artificial Sequence
  SNP primer
acgttggatgtttgtggcatgcgcctgtag

SEQ ID NO: 660 - DNA, 30 bp, Artificial Sequence
  SNP primer
acgttggatgtggcaggcagagcaatttag

SEQ ID NO: 661 - DNA, 30 bp, Artificial Sequence
  SNP primer
acgttggatgacaaaagttagctgggcgtg

SEQ ID NO: 662 - DNA, 30 bp, Artificial Sequence
  SNP primer
acgttggatgggagaggtggtcatgatcag

SEQ ID NO: 663 - DNA, 31 bp, Artificial Sequence
  SNP primer
acgttggatgcggcctatttctagtcttttg

SEQ ID NO: 664 - DNA, 30 bp, Artificial Sequence
  SNP primer
acgttggatgttgggtggtcgatgggactg

SEQ ID NO: 665 - DNA, 30 bp, Artificial Sequence
  SNP primer
acgttggatgatcttctgtggcagacgtag

SEQ ID NO: 666 - DNA, 30 bp, Artificial Sequence
  SNP primer
acgttggatggctcttgctctttcttctgg

SEQ ID NO: 667 - DNA, 30 bp, Artificial Sequence
  SNP primer
acgttggatgcctcaaggaggcagttaaac

SEQ ID NO: 668 - DNA, 30 bp, Artificial Sequence
  SNP primer
acgttggatgtcagcctcatgagtagctgg

SEQ ID NO: 669 - DNA, 30 bp, Artificial Sequence
  SNP primer
acgttggatgtgagctgtcttccttgcctc

SEQ ID NO: 670 - DNA, 31 bp, Artificial Sequence
  SNP primer
acgttggatggaaactgtcaccacaatcaag

SEQ ID NO: 671 - DNA, 31 bp, Artificial Sequence
  SNP primer
acgttggatggtgtgtatgtgtttaagagag

SEQ ID NO: 672 - DNA, 30 bp, Artificial Sequence
  SNP primer
acgttggatgcctttctgacctctctcttg

SEQ ID NO: 673 - DNA, 30 bp, Artificial Sequence
  SNP primer
acgttggatgtctaagccgttgtttatggg

SEQ ID NO: 674 - DNA, 30 bp, Artificial Sequence
  SNP primer
acgttggatgtgcatcctcatggtgagcag

SEQ ID NO: 675 - DNA, 30 bp, Artificial Sequence
  SNP primer
acgttggatgccagactgggcaacaaaatg

SEQ ID NO: 676 - DNA, 30 bp, Artificial Sequence
  SNP primer
acgttggatgtgtttagggatctgtgtggg

SEQ ID NO: 677 - DNA, 30 bp, Artificial Sequence
  SNP primer
acgttggatgtagtcatgggtagggtatgg

SEQ ID NO: 678 - DNA, 30 bp, Artificial Sequence
  SNP primer
acgttggatgtcagtggccgagaaaaacac

SEQ ID NO: 679 - DNA, 30 bp, Artificial Sequence
  SNP primer
acgttggatgatcttggcatgggccagatc

SEQ ID NO: 680 - DNA, 30 bp, Artificial Sequence
  SNP primer
acgttggatgggcctttgaggccacatcaa

SEQ ID NO: 681 - DNA, 30 bp, Artificial Sequence
  SNP primer
acgttggatgccatcctggtcttaatcttc

SEQ ID NO: 682 - DNA, 30 bp, Artificial Sequence
  SNP primer
acgttggatggattaaaccatggccactgg

SEQ ID NO: 683 - DNA, 30 bp, Artificial Sequence
  SNP primer
acgttggatgatcgagccattgcactccag

SEQ ID NO: 684 - DNA, 30 bp, Artificial Sequence
  SNP primer
acgttggatgcaacatggcagattagcatc

SEQ ID NO: 685 - DNA, 30 bp, Artificial Sequence
  SNP primer
acgttggatgtgagtcaaatgtgagggtgg

SEQ ID NO: 686 - DNA, 30 bp, Artificial Sequence
  SNP primer
acgttggatgcccactggaataacatactc

SEQ ID NO: 687 - DNA, 29 bp, Artificial Sequence
  SNP primer
acgttggatgtggtgttagagcctgcagc

SEQ ID NO: 688 - DNA, 30 bp, Artificial Sequence
  SNP primer
acgttggatgcacctccttgaatggaagtg

SEQ ID NO: 689 - DNA, 33 bp, Artificial Sequence
  SNP primer
acgttggatgctttgttttagaatttttaaaac

SEQ ID NO: 690 - DNA, 30 bp, Artificial Sequence
  SNP primer
acgttggatggccttttgagccatctcttg

SEQ ID NO: 691 - DNA, 30 bp, Artificial Sequence
  SNP primer
acgttggatgtaaaacacagcctccttggg

SEQ ID NO: 692 - DNA, 30 bp, Artificial Sequence
  SNP primer
acgttggatgaatctggtttgccagcacag

SEQ ID NO: 693 - DNA, 30 bp, Artificial Sequence
  SNP primer
acgttggatggctgttggacagtgaaatgg

SEQ ID NO: 694 - DNA, 30 bp, Artificial Sequence
  SNP primer
acgttggatgaactacagcctctgcaccag

SEQ ID NO: 695 - DNA, 30 bp, Artificial Sequence
  SNP primer
acgttggatgcttgtgcctgtaatcccaac

SEQ ID NO: 696 - DNA, 30 bp, Artificial Sequence
  SNP primer
acgttggatgtcatgtctgtcttcccttcc

SEQ ID NO: 697 - DNA, 30 bp, Artificial Sequence
  SNP primer
acgttggatgtaagcctactctcctacctc

SEQ ID NO: 698 - DNA, 30 bp, Artificial Sequence
  SNP primer
acgttggatgctgggtgacaaagcgagatc

SEQ ID NO: 699 - DNA, 30 bp, Artificial Sequence
  SNP primer
acgttggatgacaaagatgccacctccttc

SEQ ID NO: 700 - DNA, 30 bp, Artificial Sequence
  SNP primer
acgttggatgggtgaccctggttgagaatc

SEQ ID NO: 701 - DNA, 30 bp, Artificial Sequence
  SNP primer
acgttggatggcactgagtgtgctgcagag

SEQ ID NO: 702 - DNA, 30 bp, Artificial Sequence
  SNP primer
acgttggatgtcatcttctgctgcagcgag

SEQ ID NO: 703 - DNA, 30 bp, Artificial Sequence; identical to SEQ ID NO: 702
  SNP primer

SEQ ID NO: 704 - DNA, 30 bp, Artificial Sequence; identical to SEQ ID NO: 702
  SNP primer

SEQ ID NO: 705 - DNA, 30 bp, Artificial Sequence
  SNP primer
acgttggatgccattcctccctcaaacttc

SEQ ID NO: 706 - DNA, 30 bp, Artificial Sequence
  SNP primer
acgttggatgattctggtcctgaccctcac

SEQ ID NO: 707 - DNA, 30 bp, Artificial Sequence
  SNP primer
acgttggatgtagagctcacgggctaactg

SEQ ID NO: 708 - DNA, 30 bp, Artificial Sequence
  SNP primer
acgttggatgcctgagggtaaaaacagtgc

SEQ ID NO: 709 - DNA, 30 bp, Artificial Sequence
  SNP primer
acgttggatgtcctacctcctcccaaaatg

SEQ ID NO: 710 - DNA, 30 bp, Artificial Sequence
  SNP primer
acgttggatgagtgcatgctgggtacatgg

SEQ ID NO: 711 - DNA, 30 bp, Artificial Sequence
  SNP primer
acgttggatgttgaaacctcctgcatcctg

SEQ ID NO: 712 - DNA, 30 bp, Artificial Sequence
  SNP primer
acgttggatggctgcacaaacaactttcgc

SEQ ID NO: 713 - DNA, 31 bp, Artificial Sequence
  SNP primer
acgttggatggtccattgaatggagtatagc

SEQ ID NO: 714 - DNA, 30 bp, Artificial Sequence
  SNP primer
acgttggatgtccttgtcagggaccacaag

SEQ ID NO: 715 - DNA, 30 bp, Artificial Sequence
  SNP primer
acgttggatgatcgtgccattgcactctag

SEQ ID NO: 716 - DNA, 30 bp, Artificial Sequence
  SNP primer
acgttggatgtggcacagtcagaataaggc

SEQ ID NO: 717 - DNA, 30 bp, Artificial Sequence
  SNP primer
acgttggatgaatctcagcaccttgagagg

SEQ ID NO: 718 - DNA, 31 bp, Artificial Sequence
  SNP primer
acgttggatgcagctttatcaggttggtttc

SEQ ID NO: 719 - DNA, 30 bp, Artificial Sequence
  SNP primer
acgttggatgtttgcctgaaggactgaaac

SEQ ID NO: 720 - DNA, 30 bp, Artificial Sequence
  SNP primer
acgttggatgggcaatacagctgcattcag

SEQ ID NO: 721 - DNA, 30 bp, Artificial Sequence
  SNP primer
acgttggatgtacaatccttcccaaggtgg

SEQ ID NO: 722 - DNA, 30 bp, Artificial Sequence
  SNP primer
acgttggatgttatgtgaccccctcaaagg

SEQ ID NO: 723 - DNA, 30 bp, Artificial Sequence
  SNP primer
acgttggatgtcaggaacactgagagaatg

SEQ ID NO: 724 - DNA, 30 bp, Artificial Sequence
  SNP primer
acgttggatgagcctcaccagcatctattg

SEQ ID NO: 725 - DNA, 30 bp, Artificial Sequence
  SNP primer
acgttggatgaggtcaatcttctccagagc

SEQ ID NO: 726 - DNA, 30 bp, Artificial Sequence
  SNP primer
acgttggatgaagttcattgacaccccctc

SEQ ID NO: 727 - DNA, 30 bp, Artificial Sequence
  SNP primer
acgttggatgtgagactcaagactggctag

SEQ ID NO: 728 - DNA, 30 bp, Artificial Sequence
  SNP primer
acgttggatgtaactcctctttctgggcag

SEQ ID NO: 729 - DNA, 30 bp, Artificial Sequence
  SNP primer
acgttggatgttctccaagtgtggacacag

SEQ ID NO: 730 - DNA, 30 bp, Artificial Sequence
  SNP primer
acgttggatgtcaaggtttctttctccaag

SEQ ID NO: 731 - DNA, 30 bp, Artificial Sequence
  SNP primer
acgttggatgttgcagtgctcctgctccca

SEQ ID NO: 732 - DNA, 30 bp, Artificial Sequence
  SNP primer
acgttggatgagaaatcccaaggagaggcc

SEQ ID NO: 733 - DNA, 30 bp, Artificial Sequence
  SNP primer
acgttggatgtttcaaggaagaatctatag

SEQ ID NO: 734 - DNA, 30 bp, Artificial Sequence
  SNP primer
acgttggatgggaaatgcaagaatgagagc

SEQ ID NO: 735 - DNA, 30 bp, Artificial Sequence
  SNP primer
acgttggatgacagctacgggtctatcaag

SEQ ID NO: 736 - DNA, 30 bp, Artificial Sequence
  SNP primer
acgttggatgttggatcagcagctcttttg

SEQ ID NO: 737 - DNA, 30 bp, Artificial Sequence
  SNP primer
acgttggatgctgcagtggtagaagagaag

SEQ ID NO: 738 - DNA, 30 bp, Artificial Sequence
  SNP primer
acgttggatgagggctgtctaatcattccc

SEQ ID NO: 739 - DNA, 30 bp, Artificial Sequence
  SNP primer
acgttggatgtacaggtctgtcccaccatc

SEQ ID NO: 740 - DNA, 30 bp, Artificial Sequence
  SNP primer
acgttggatgtctcctctcacaccatccag

SEQ ID NO: 741 - DNA, 30 bp, Artificial Sequence
  SNP primer
acgttggatgttcctccgtatctgtctgtg

SEQ ID NO: 742 - DNA, 30 bp, Artificial Sequence
  SNP primer
acgttggatgaaaaagagtagagcccctgg

SEQ ID NO: 743 - DNA, 30 bp, Artificial Sequence
  SNP primer
acgttggatgtgggagggccttgaaaatac

SEQ ID NO: 744 - DNA, 30 bp, Artificial Sequence
  SNP primer
acgttggatgttctatctcctgctggtctg

SEQ ID NO: 745 - DNA, 31 bp, Artificial Sequence
  SNP primer
acgttggatgcgatagttctgaaatcgtagg

SEQ ID NO: 746 - DNA, 30 bp, Artificial Sequence
  SNP primer
acgttggatggagtggaagagcaagacaac

SEQ ID NO: 747 - DNA, 30 bp, Artificial Sequence
  SNP primer
acgttggatggccaccccagatctttattc

SEQ ID NO: 748 - DNA, 30 bp, Artificial Sequence
  SNP primer
acgttggatggtccattgttcttcaggaac

SEQ ID NO: 749 - DNA, 30 bp, Artificial Sequence
  SNP primer
acgttggatgagcagccttccatagacatc

SEQ ID NO: 750 - DNA, 30 bp, Artificial Sequence
  SNP primer
acgttggatgagccaccacaccttcttctg

SEQ ID NO: 751 - DNA, 30 bp, Artificial Sequence
  SNP primer
acgttggatgtaggaaacatccccacctag

SEQ ID NO: 752 - DNA, 30 bp, Artificial Sequence
  SNP primer
acgttggatgatgaaaatgcagacccgcag

SEQ ID NO: 753 - DNA, 30 bp, Artificial Sequence
  SNP primer
acgttggatgtctcagcggctcaagcagtg

SEQ ID NO: 754 - DNA, 30 bp, Artificial Sequence
  SNP primer
acgttggatgtgctggtgagtggcgttcct

SEQ ID NO: 755 - DNA, 30 bp, Artificial Sequence
  SNP primer
acgttggatgactatttgctccctctgagg

SEQ ID NO: 756 - DNA, 30 bp, Artificial Sequence
  SNP primer
acgttggatgtgagcccagagttgacagag

SEQ ID NO: 757 - DNA, 30 bp, Artificial Sequence
  SNP primer
acgttggatgtagtaaagagggcaggcatg

SEQ ID NO: 758 - DNA, 30 bp, Artificial Sequence
  SNP primer
acgttggatgaaagacagcacattctgccg

SEQ ID NO: 759 - DNA, 29 bp, Artificial Sequence
  SNP primer
acgttggatgtcagctccccgcccagcca

SEQ ID NO: 760 - DNA, 29 bp, Artificial Sequence
  SNP primer
acgttggatgtgaggagcgtctccgcccg

SEQ ID NO: 761 - DNA, 30 bp, Artificial Sequence
  SNP primer
acgttggatgtatcaattcgcccattgcag

SEQ ID NO: 762 - DNA, 30 bp, Artificial Sequence
  SNP primer
acgttggatgttgcagtgagctgagattgc

SEQ ID NO: 763 - DNA, 30 bp, Artificial Sequence
  SNP primer
acgttggatgaaacccaaagatgggtcagg

SEQ ID NO: 764 - DNA, 30 bp, Artificial Sequence
  SNP primer
acgttggatgaactgccattagcatcaggg

SEQ ID NO: 765 - DNA, 30 bp, Artificial Sequence
  SNP primer
acgttggatggggaggaagaaaaaagtggg

SEQ ID NO: 766 - DNA, 30 bp, Artificial Sequence
  SNP primer
acgttggatgtcaacggtgtttcttggagg

SEQ ID NO: 767 - DNA, 30 bp, Artificial Sequence
  SNP primer
acgttggatgccatgcctggctaatatttg

SEQ ID NO: 768 - DNA, 30 bp, Artificial Sequence
  SNP primer
acgttggatgcgtgtgcattagcaaagtgg

SEQ ID NO: 769 - DNA, 30 bp, Artificial Sequence
  SNP primer
acgttggatgttgctatacttcgggtcacg

SEQ ID NO: 770 - DNA, 30 bp, Artificial Sequence
  SNP primer
acgttggatgtttcgccatgttggacaagc

SEQ ID NO: 771 - DNA, 30 bp, Artificial Sequence
  SNP primer
acgttggatggggtctccccatccttattc

SEQ ID NO: 772 - DNA, 30 bp, Artificial Sequence
  SNP primer
acgttggatggtggagaaagactgagctag

SEQ ID NO: 773 - DNA, 30 bp, Artificial Sequence
  SNP primer
acgttggatgacacaaatcagtcagtggcc

SEQ ID NO: 774 - DNA, 30 bp, Artificial Sequence
  SNP primer
acgttggatgaaacacatggctcacccttc

SEQ ID NO: 775 - DNA, 30 bp, Artificial Sequence
  SNP primer
acgttggatgtgatctcatctttcccccac

SEQ ID NO: 776 - DNA, 30 bp, Artificial Sequence
  SNP primer
acgttggatgaagtgattctcctgcctcag

SEQ ID NO: 777 - DNA, 30 bp, Artificial Sequence
  SNP primer
acgttggatgagatgctggagtggcctctg

SEQ ID NO: 778 - DNA, 30 bp, Artificial Sequence
  SNP primer
acgttggatgagacaacgtggaaggaggag

SEQ ID NO: 779 - DNA, 30 bp, Artificial Sequence
  SNP primer
acgttggatgcttttcccacctagttctgg

SEQ ID NO: 780 - DNA, 30 bp, Artificial Sequence
  SNP primer
acgttggatgtgttagcttctgagggatgg

SEQ ID NO: 781 - DNA, 30 bp, Artificial Sequence
  SNP primer
acgttggatgtcagttctctcagcttctgg

SEQ ID NO: 782 - DNA, 30 bp, Artificial Sequence
  SNP primer
acgttggatgagtctttcgggtacactctg

SEQ ID NO: 783 - DNA, 30 bp, Artificial Sequence
  SNP primer
acgttggatgagatgactcaccggatactg

SEQ ID NO: 784 - DNA, 30 bp, Artificial Sequence
  SNP primer
acgttggatgtactggactcaatgagcagg

SEQ ID NO: 785 - DNA, 30 bp, Artificial Sequence
  SNP primer
acgttggatggggaatgagaaaaggaactg

SEQ ID NO: 786 - DNA, 30 bp, Artificial Sequence
  SNP primer
acgttggatgaaagcactagcaccttcagc

SEQ ID NO: 787 - DNA, 30 bp, Artificial Sequence
  SNP primer
acgttggatgacagagagagactctgtctc

SEQ ID NO: 788 - DNA, 30 bp, Artificial Sequence
  SNP primer
acgttggatgaggtaagggtagagctgttg

SEQ ID NO: 789 - DNA, 30 bp, Artificial Sequence
  SNP primer
acgttggatgagaagccatcagtgagtcac

SEQ ID NO: 790 - DNA, 30 bp, Artificial Sequence
  SNP primer
acgttggatgacgagcctgcaaaaggagcg

SEQ ID NO: 791 - DNA, 30 bp, Artificial Sequence
  SNP primer
acgttggatgttacctggagatgacctcag

SEQ ID NO: 792 - DNA, 30 bp, Artificial Sequence
  SNP primer
acgttggatggttatgcatatgcacagatg

SEQ ID NO: 793 - DNA, 30 bp, Artificial Sequence
  SNP primer
acgttggatgcctgcactgacaagtatgac

SEQ ID NO: 794 - DNA, 30 bp, Artificial Sequence
  SNP primer
acgttggatgaaaacctaggatcatgggcc

SEQ ID NO: 795 - DNA, 30 bp, Artificial Sequence
  SNP primer
acgttggatgactctttttgctcaggctgg

SEQ ID NO: 796 - DNA, 30 bp, Artificial Sequence
  SNP primer
acgttggatgtggtgaagtctggtagcatg

SEQ ID NO: 797 - DNA, 30 bp, Artificial Sequence
  SNP primer
acgttggatgagtctgagaaggtacaggac

SEQ ID NO: 798 - DNA, 30 bp, Artificial Sequence
  SNP primer
acgttggatgtgtgtaaccccatcatcagc

SEQ ID NO: 799 - DNA, 30 bp, Artificial Sequence
  SNP primer
acgttggatgcacctccttcctactttctc

SEQ ID NO: 800 - DNA, 30 bp, Artificial Sequence
  SNP primer
acgttggatggggcacttcaagtagatagc

SEQ ID NO: 801 - DNA, 30 bp, Artificial Sequence
  SNP primer
acgttggatgggagagtctgtaagaagcag

SEQ ID NO: 802 - DNA, 30 bp, Artificial Sequence
  SNP primer
acgttggatggtgattggaaaggaggtctc

SEQ ID NO: 803 - DNA, 30 bp, Artificial Sequence
  SNP primer
acgttggatgactgctctgtatcctctgcc

SEQ ID NO: 804 - DNA, 30 bp, Artificial Sequence
  SNP primer
acgttggatgacctggcatgctctgataag

SEQ ID NO: 805 - DNA, 30 bp, Artificial Sequence
  SNP primer
acgttggatgtgtccaggcatggagtagtg

SEQ ID NO: 806 - DNA, 30 bp, Artificial Sequence
  SNP primer
acgttggatgaggctgaggcaggagaattg

SEQ ID NO: 807 - DNA, 30 bp, Artificial Sequence
  SNP primer
acgttggatgtgcatccgtggcacctctca

SEQ ID NO: 808 - DNA, 30 bp, Artificial Sequence
  SNP primer
acgttggatgtgtcccatctacattcctag

SEQ ID NO: 809 - DNA, 30 bp, Artificial Sequence
  SNP primer
acgttggatgtgacccacagaagtcttttc

SEQ ID NO: 810 - DNA, 30 bp, Artificial Sequence
  SNP primer
acgttggatgaatctcggcactttgggagg

SEQ ID NO: 811 - DNA, 30 bp, Artificial Sequence
  SNP primer
acgttggatgcctccaagaagtcttctcag

SEQ ID NO: 812 - DNA, 30 bp, Artificial Sequence
  SNP primer
acgttggatgtgattacaggtgtgagccac

SEQ ID NO: 813 - DNA, 30 bp, Artificial Sequence
  SNP primer
acgttggatgatagactgcgtcacttcagc

SEQ ID NO: 814 - DNA, 30 bp, Artificial Sequence
  SNP primer
acgttggatgttgttcaccagtgtgcagtg

SEQ ID NO: 815 - DNA, 30 bp, Artificial Sequence
  SNP primer
acgttggatgagagtgtgctgtctgaggag

SEQ ID NO: 816 - DNA, 30 bp, Artificial Sequence
  SNP primer
acgttggatgtgtctgcacaaggagagaag

SEQ ID NO: 817 - DNA, 30 bp, Artificial Sequence
  SNP primer
acgttggatgggaaaagaaaaagtggaggg

SEQ ID NO: 818 - DNA, 30 bp, Artificial Sequence
  SNP primer
acgttggatgcaagaagagagggacaggag

SEQ ID NO: 819 - DNA, 30 bp, Artificial Sequence
  SNP primer
acgttggatggggagaagacaggggattag

SEQ ID NO: 820 - DNA, 30 bp, Artificial Sequence
  SNP primer
acgttggatgcgtggaagccgtacaggttc

SEQ ID NO: 821 - DNA, 30 bp, Artificial Sequence
  SNP primer
acgttggatgatcccagcactttgtaaggc

SEQ ID NO: 822 - DNA, 30 bp, Artificial Sequence
  SNP primer
acgttggatgctccaactccaccacaaaac

SEQ ID NO: 823 - DNA, 30 bp, Artificial Sequence
  SNP primer
acgttggatgtgtcaggcactgaccaagtc

SEQ ID NO: 824 - DNA, 30 bp, Artificial Sequence
  SNP primer
acgttggatgaatggaagccacctgaccac

SEQ ID NO: 825 - DNA, 30 bp, Artificial Sequence
  SNP primer
acgttggatgatgtgttggccggggtacac

SEQ ID NO: 826 - DNA, 30 bp, Artificial Sequence
  SNP primer
acgttggatgatacagagagctgccctttc

SEQ ID NO: 827 - DNA, 30 bp, Artificial Sequence
  SNP primer
acgttggatgcaggatgaaagatgggagag

SEQ ID NO: 828 - DNA, 30 bp, Artificial Sequence
  SNP primer
acgttggatgctgaggaatcatcagaggtg

SEQ ID NO: 829 - DNA, 30 bp, Artificial Sequence
  SNP primer
acgttggatggtaacagaggcagctgtttg

SEQ ID NO: 830 - DNA, 29 bp, Artificial Sequence
  SNP primer
acgttggatgttcgtggtgcccacgcacg

SEQ ID NO: 831 - DNA, 30 bp, Artificial Sequence
  SNP primer
acgttggatgacagccacttcaagtagtcc

SEQ ID NO: 832 - DNA, 30 bp, Artificial Sequence
  SNP primer
acgttggatggctgtatccacattcacttc

SEQ ID NO: 833 - DNA, 30 bp, Artificial Sequence
  SNP primer
acgttggatgaagctgcttacagcctaagg

SEQ ID NO: 834 - DNA, 30 bp, Artificial Sequence
  SNP primer
acgttggatggctctcttggcagaatttgg

SEQ ID NO: 835 - DNA, 30 bp, Artificial Sequence
  SNP primer
acgttggatggcacctacttcatagggttg

SEQ ID NO: 836 - DNA, 30 bp, Artificial Sequence
  SNP primer
acgttggatgtaaggaactcgagttggcag

SEQ ID NO: 837 - DNA, 30 bp, Artificial Sequence
  SNP primer
acgttggatgcactggcctgtaattatggg

SEQ ID NO: 838 - DNA, 30 bp, Artificial Sequence
  SNP primer
acgttggatgacctgctcattttctccaac

SEQ ID NO: 839 - DNA, 30 bp, Artificial Sequence
  SNP primer
acgttggatgtgacgaggttcttcctggag

SEQ ID NO: 840 - DNA, 30 bp, Artificial Sequence
  SNP primer
acgttggatgatgccccaatcatctctcac

SEQ ID NO: 841 - DNA, 31 bp, Artificial Sequence
  SNP primer
acgttggatgcaaggagactactatcatcac

SEQ ID NO: 842 - DNA, 30 bp, Artificial Sequence
  SNP primer
acgttggatgttgattccattctccccgtc

SEQ ID NO: 843 - DNA, 30 bp, Artificial Sequence
  SNP primer
acgttggatgcagcatcactaaggatcagg

SEQ ID NO: 844 - DNA, 30 bp, Artificial Sequence
  SNP primer
acgttggatgttacactcccatcaacagtg

SEQ ID NO: 845 - DNA, 30 bp, Artificial Sequence
  SNP primer
acgttggatgtcccactaggtctttgaagg

SEQ ID NO: 846 - DNA, 30 bp, Artificial Sequence
  SNP primer
acgttggatgtggtgtatttagacctacac

SEQ ID NO: 847 - DNA, 30 bp, Artificial Sequence
  SNP primer
acgttggatgtgtgctacccatgcagtgtg

SEQ ID NO: 848 - DNA, 30 bp, Artificial Sequence
  SNP primer
acgttggatggcccccaaggaataagaaac

SEQ ID NO: 849 - DNA, 30 bp, Artificial Sequence
  SNP primer
acgttggatggggtgcatactactttaacc

SEQ ID NO: 850 - DNA, 30 bp, Artificial Sequence
  SNP primer
acgttggatgtgacacatcattctctcacc

SEQ ID NO: 851 - DNA, 30 bp, Artificial Sequence
  SNP primer
acgttggatgaagcaagagatggctagtgc

SEQ ID NO: 852 - DNA, 30 bp, Artificial Sequence
  SNP primer
acgttggatgctgtcaaagcaagagatggc

SEQ ID NO: 853 - DNA, 30 bp, Artificial Sequence
  SNP primer
acgttggatgttctttgtaccagctaggcc

SEQ ID NO: 854 - DNA, 30 bp, Artificial Sequence
  SNP primer
acgttggatgagaacagtcccttgagaaag

SEQ ID NO: 855 - DNA, 30 bp, Artificial Sequence
  SNP primer
acgttggatgtttctagctctgatcgcctg

SEQ ID NO: 856 - DNA, 30 bp, Artificial Sequence
  SNP primer
acgttggatgagggaaatgagtatgttgag

SEQ ID NO: 857 - DNA, 30 bp, Artificial Sequence
  SNP primer
acgttggatgcagtcacttagaggatcttg

SEQ ID NO: 858 - DNA, 30 bp, Artificial Sequence
  SNP primer
acgttggatggggcagtggaaggtatttac

SEQ ID NO: 859 - DNA, 30 bp, Artificial Sequence
  SNP primer
acgttggatgaaaccctgtcattgatcgac

SEQ ID NO: 860 - DNA, 30 bp, Artificial Sequence
  SNP primer
acgttggatgatctcttccattgctgccag

SEQ ID NO: 861 - DNA, 30 bp, Artificial Sequence
  SNP primer
acgttggatgctgaggacacagctaatatc

SEQ ID NO: 862 - DNA, 30 bp, Artificial Sequence
  SNP primer
acgttggatgctaactttttgctcttatcc

SEQ ID NO: 863 - DNA, 30 bp, Artificial Sequence
  SNP primer
acgttggatgtgaattctcagtcagctgag

SEQ ID NO: 864 - DNA, 30 bp, Artificial Sequence
  SNP primer
acgttggatgaaatggtgcctcatagcgtg

SEQ ID NO: 865 - DNA, 30 bp, Artificial Sequence
  SNP primer
acgttggatgcaggttttgaaacgtgagag

SEQ ID NO: 866 - DNA, 30 bp, Artificial Sequence
  SNP primer
acgttggatggcaggaactaggaattgtgc

SEQ ID NO: 867 - DNA, 30 bp, Artificial Sequence
  SNP primer
acgttggatgcaagtagtcaatatgccctc

SEQ ID NO: 868 - DNA, 30 bp, Artificial Sequence
  SNP primer
acgttggatgagtgtctgaaatgccctgtc

SEQ ID NO: 869 - DNA, 30 bp, Artificial Sequence
  SNP primer
acgttggatgtaactgtgggttgtttcccc

SEQ ID NO: 870 - DNA, 30 bp, Artificial Sequence
  SNP primer
acgttggatgtatgaacttgggtgtcaggg

SEQ ID NO: 871 - DNA, 30 bp, Artificial Sequence
  SNP primer
acgttggatggtacgtctaactgctgttcg

SEQ ID NO: 872 - DNA, 30 bp, Artificial Sequence
  SNP primer
acgttggatgtagatgagagtgcaacttcg

SEQ ID NO: 873 - DNA, 30 bp, Artificial Sequence
  SNP primer
acgttggatgaaatgtttgtgccatctaag

SEQ ID NO: 874 - DNA, 30 bp, Artificial Sequence; identical to SEQ ID NO: 334
  SNP primer

SEQ ID NO: 875 - DNA, 30 bp, Artificial Sequence
  SNP primer
acgttggatgcccatcctgaaaaccttacc

SEQ ID NO: 876 - DNA, 30 bp, Artificial Sequence
  SNP primer
acgttggatgagaccaactcagacttactc

SEQ ID NO: 877 - DNA, 30 bp, Artificial Sequence
  SNP primer
acgttggatgaggaaatctgagtatcttac

SEQ ID NO: 878 - DNA, 30 bp, Artificial Sequence
  SNP primer
acgttggatgattaaagtggcaggagcagg

SEQ ID NO: 879 - DNA, 30 bp, Artificial Sequence
  SNP primer
acgttggatggagagcagatggcagagtac

SEQ ID NO: 880 - DNA, 30 bp, Artificial Sequence
  SNP primer
acgttggatgtaagtcatggtaacctccgg

SEQ ID NO: 881 - DNA, 30 bp, Artificial Sequence
  SNP primer
acgttggatgtcctctgagttctctgagac

SEQ ID NO: 882 - DNA, 30 bp, Artificial Sequence
  SNP primer
acgttggatgtggctttcaatacatattgc

SEQ ID NO: 883 - DNA, 31 bp, Artificial Sequence
  SNP primer
acgttggatgaggtcagtgctctgcctctag

SEQ ID NO: 884 - DNA, 30 bp, Artificial Sequence
  SNP primer
acgttggatgtggcttccgcatagaacagg

SEQ ID NO: 885 - DNA, 30 bp, Artificial Sequence
  SNP primer
acgttggatgggcctctgagatgtagttac

SEQ ID NO: 886 - DNA, 30 bp, Artificial Sequence
  SNP primer
acgttggatggaaatctgctaggcttaaac

SEQ ID NO: 887 - DNA, 30 bp, Artificial Sequence
  SNP primer
acgttggatgtcacctggacctgattgatc

SEQ ID NO: 888 - DNA, 30 bp, Artificial Sequence
  SNP primer
acgttggatggggctggataatgatggatg

SEQ ID NO: 889 - DNA, 30 bp, Artificial Sequence
  SNP primer
acgttggatgtttgtccctctagccctttg

SEQ ID NO: 890 - DNA, 30 bp, Artificial Sequence
  SNP primer
acgttggatgccatagaaaccttggaagtc

SEQ ID NO: 891 - DNA, 30 bp, Artificial Sequence
  SNP primer
acgttggatgggagcctcaaattatcactc

SEQ ID NO: 892 - DNA, 30 bp, Artificial Sequence
  SNP primer
acgttggatgtcttctatatcggataatgc

SEQ ID NO: 893 - DNA, 30 bp, Artificial Sequence; identical to SEQ ID NO: 892
  SNP primer

SEQ ID NO: 894 - DNA, 30 bp, Artificial Sequence
  SNP primer
acgttggatggccacatgatcatgaaggtg

SEQ ID NO: 895 - DNA, 30 bp, Artificial Sequence
  SNP primer
acgttggatgtcaccttcatgatcatgtgg

SEQ ID NO: 896 - DNA, 30 bp, Artificial Sequence
  SNP primer
acgttggatgtgagaagatgagagcaacag

SEQ ID NO: 897 - DNA, 30 bp, Artificial Sequence
  SNP primer
acgttggatggaggtgctgagggaatattg

SEQ ID NO: 898 - DNA, 30 bp, Artificial Sequence
  SNP primer
acgttggatgacttccacctcccaaatctc

SEQ ID NO: 899 - DNA, 30 bp, Artificial Sequence; identical to SEQ ID NO: 898
  SNP primer

SEQ ID NO: 900 - DNA, 30 bp, Artificial Sequence
  SNP primer
acgttggatgagcccttaaaactgttaggg

SEQ ID NO: 901 - DNA, 30 bp, Artificial Sequence
  SNP primer
acgttggatggcttggagcatcaaactctg

SEQ ID NO: 902 - DNA, 30 bp, Artificial Sequence
  SNP primer
acgttggatgaggaacagatgtggctcttg

SEQ ID NO: 903 - DNA, 30 bp, Artificial Sequence
  SNP primer
acgttggatgggaaaaggcaatagacaggg

SEQ ID NO: 904 - DNA, 31 bp, Artificial Sequence
  SNP primer
acgttggatggcagaagtttcttcagtgatc

SEQ ID NO: 905 - DNA, 30 bp, Artificial Sequence
  SNP primer
acgttggatgtgctttcactgaggtcaagg

SEQ ID NO: 906 - DNA, 30 bp, Artificial Sequence
  SNP primer
acgttggatgttgggagatctctactgagc

SEQ ID NO: 907 - DNA, 30 bp, Artificial Sequence
  SNP primer
acgttggatgttccttctccccactctaac

SEQ ID NO: 908 - DNA, 30 bp, Artificial Sequence
  SNP primer
acgttggatggaaggaagacagaagaatcc

SEQ ID NO: 909 - DNA, 30 bp, Artificial Sequence
  SNP primer
acgttggatgtcagtcagactactctctcg

SEQ ID NO: 910 - DNA, 30 bp, Artificial Sequence
  SNP primer
acgttggatgtggtttatggcaggttctag

SEQ ID NO: 911 - DNA, 30 bp, Artificial Sequence
  SNP primer
acgttggatgcaaacctgtacatgtatccc

SEQ ID NO: 912 - DNA, 30 bp, Artificial Sequence
  SNP primer
acgttggatgatttcagagatttcgggatc

SEQ ID NO: 913 - DNA, 30 bp, Artificial Sequence
  SNP primer
acgttggatgttccagggagacagaatgtg

SEQ ID NO: 914 - DNA, 30 bp, Artificial Sequence
  SNP primer
acgttggatgagtacacagtagctgctgag

SEQ ID NO: 915 - DNA, 30 bp, Artificial Sequence
  SNP primer
acgttggatgactacatttgggatactggg

SEQ ID NO: 916 - DNA, 30 bp, Artificial Sequence
  SNP primer
acgttggatggtaccttactccctgtgttg

SEQ ID NO: 917 - DNA, 30 bp, Artificial Sequence
  SNP primer
acgttggatgagccactgagggagtagtgg

SEQ ID NO: 918 - DNA, 30 bp, Artificial Sequence
  SNP primer
acgttggatgggtgtgggttttggtgttag

SEQ ID NO: 919 - DNA, 30 bp, Artificial Sequence
  SNP primer
acgttggatggtggaagaggatacataggg

SEQ ID NO: 920 - DNA, 30 bp, Artificial Sequence
  SNP primer
acgttggatgctggaactgaagctgagatc

SEQ ID NO: 921 - DNA, 30 bp, Artificial Sequence
  SNP primer
acgttggatgataacccaggatcgtctagg

SEQ ID NO: 922 - DNA, 30 bp, Artificial Sequence
  SNP primer
acgttggatgcgttgacagtgacactgatg

SEQ ID NO: 923 - DNA, 30 bp, Artificial Sequence
  SNP primer
acgttggatggctgtatgaaggtcctcttc

SEQ ID NO: 924 - DNA, 30 bp, Artificial Sequence
  SNP primer
acgttggatgtaggccttagtgtttccagg

SEQ ID NO: 925 - DNA, 30 bp, Artificial Sequence
  SNP primer
acgttggatgcagaatggacttcaaagtac

SEQ ID NO: 926 - DNA, 31 bp, Artificial Sequence
  SNP primer
acgttggatgggaactagaattgctacacag

SEQ ID NO: 927 - DNA, 30 bp, Artificial Sequence
  SNP primer
acgttggatgacagtgacctcaaaattccc

SEQ ID NO: 928 - DNA, 30 bp, Artificial Sequence
  SNP primer
acgttggatgaactgggcttcctcagcagg

SEQ ID NO: 929 - DNA, 30 bp, Artificial Sequence
  SNP primer
acgttggatgttgggtgtgatctgctccac

SEQ ID NO: 930 - DNA, 30 bp, Artificial Sequence
  SNP primer
acgttggatgtccatgcagcaagatccagg

SEQ ID NO: 931 - DNA, 31 bp, Artificial Sequence
  SNP primer
acgttggatgttgccatgagcagcctaggtg

SEQ ID NO: 932 - DNA, 30 bp, Artificial Sequence
  SNP primer
acgttggatgcttttacaggcctcagaggg

SEQ ID NO: 933 - DNA, 30 bp, Artificial Sequence
  SNP primer
acgttggatgttactgtgaacatcagggcc

SEQ ID NO: 934 - DNA, 30 bp, Artificial Sequence
  SNP primer
acgttggatgtaaactagttctcctactcc

SEQ ID NO: 935 - DNA, 30 bp, Artificial Sequence
  SNP primer
acgttggatgacagagcaagatgctgagtg

SEQ ID NO: 936 - DNA, 30 bp, Artificial Sequence
  SNP primer
acgttggatgtaatccctgcactttgggag

SEQ ID NO: 937 - DNA, 30 bp, Artificial Sequence
  SNP primer
acgttggatgatgctggagaacaggacagg

SEQ ID NO: 938 - DNA, 30 bp, Artificial Sequence
  SNP primer
acgttggatgtctagacactggtaaccctg

SEQ ID NO: 939 - DNA, 30 bp, Artificial Sequence
  SNP primer
acgttggatgaggagcagagaaaagtcctg

SEQ ID NO: 940 - DNA, 30 bp, Artificial Sequence
  SNP primer
acgttggatgactgggcttgaacagtgatg

SEQ ID NO: 941 - DNA, 30 bp, Artificial Sequence
  SNP primer
acgttggatgtgtcctccttggaaaatgag

SEQ ID NO: 942 - DNA, 30 bp, Artificial Sequence
  SNP primer
acgttggatgaaaccagagtcccaacctac

SEQ ID NO: 943 - DNA, 30 bp, Artificial Sequence
  SNP primer
acgttggatggcaaagcttcattgaggagg

SEQ ID NO: 944 - DNA, 30 bp, Artificial Sequence
  SNP primer
acgttggatgtggtaagcatcactgtctcc

SEQ ID NO: 945 - DNA, 30 bp, Artificial Sequence
  SNP primer
acgttggatgaagggtcactatatctgccc

SEQ ID NO: 946 - DNA, 30 bp, Artificial Sequence
  SNP primer
acgttggatgaggaggtagaagatcaactc

SEQ ID NO: 947 - DNA, 30 bp, Artificial Sequence
  SNP primer
acgttggatgcatgaatgatgcgacaactg

SEQ ID NO: 948 - DNA, 30 bp, Artificial Sequence
  SNP primer
acgttggatgcagttcctcatggacatatc

SEQ ID NO: 949 - DNA, 30 bp, Artificial Sequence
  SNP primer
acgttggatgacatttgagggcaaatgagg

SEQ ID NO: 950 - DNA, 30 bp, Artificial Sequence
  SNP primer
acgttggatgactcacagaagccaaagaag

SEQ ID NO: 951 - DNA, 32 bp, Artificial Sequence
  SNP primer
acgttggatgccaaagcatttaatggtttaag

SEQ ID NO: 952 - DNA, 30 bp, Artificial Sequence
  SNP primer
acgttggatggttatgggtgttgtttagcc

SEQ ID NO: 953 - DNA, 31 bp, Artificial Sequence
  SNP primer
acgttggatgccccttaagatggtctaatag

SEQ ID NO: 954 - DNA, 30 bp, Artificial Sequence
  SNP primer
acgttggatgaacacatcagaaggcacacc

SEQ ID NO: 955 - DNA, 30 bp, Artificial Sequence
  SNP primer
acgttggatggacccttgggaaatttctac

SEQ ID NO: 956 - DNA, 30 bp, Artificial Sequence
  SNP primer
acgttggatgacatcactctactggcccag

SEQ ID NO: 957 - DNA, 30 bp, Artificial Sequence
  SNP primer
acgttggatgtcataagagcccttggtgtc

SEQ ID NO: 958 - DNA, 30 bp, Artificial Sequence
  SNP primer
acgttggatgctggcttccatttcttttcc

SEQ ID NO: 959 - DNA, 31 bp, Artificial Sequence
  SNP primer
acgttggatgtttcagtgagatcctgggtta

SEQ ID NO: 960 - DNA, 30 bp, Artificial Sequence
  SNP primer
acgttggatgtgagctgtgttgactaccac

SEQ ID NO: 961 - DNA, 30 bp, Artificial Sequence
  SNP primer
acgttggatggaagattgaatggcttaggg

SEQ ID NO: 962 - DNA, 30 bp, Artificial Sequence
  SNP primer
acgttggatgactccagaagagtagagtgg

SEQ ID NO: 963 - DNA, 30 bp, Artificial Sequence
  SNP primer
acgttggatggtagtgaacttccatgcagg

SEQ ID NO: 964 - DNA, 30 bp, Artificial Sequence
  SNP primer
acgttggatgcgagtaggtactctcattgg

SEQ ID NO: 965 - DNA, 30 bp, Artificial Sequence
  SNP primer
acgttggatggcggattcctaattctgagg

SEQ ID NO: 966 - DNA, 30 bp, Artificial Sequence
  SNP primer
acgttggatgaagtgtatcccattttttcc

SEQ ID NO: 967 - DNA, 30 bp, Artificial Sequence
  SNP primer
acgttggatgtctgaagctgcacctggagg

SEQ ID NO: 968 - DNA, 30 bp, Artificial Sequence
  SNP primer
acgttggatggaaacaactccaggcatttg

SEQ ID NO: 969 - DNA, 30 bp, Artificial Sequence
  SNP primer
acgttggatgatgttaccttggcgaaaggc

SEQ ID NO: 970 - DNA, 30 bp, Artificial Sequence
  SNP primer
acgttggatgctatgaacaattctacacac

SEQ ID NO: 971 - DNA, 30 bp, Artificial Sequence
  SNP primer
acgttggatgaggcttaatgatgagaggtg

SEQ ID NO: 972 - DNA, 30 bp, Artificial Sequence
  SNP primer
acgttggatgtgcaactatctggacacatg

SEQ ID NO: 973 - DNA, 30 bp, Artificial Sequence
  SNP primer
acgttggatgccatgagtgaggattccaag

SEQ ID NO: 974 - DNA, 30 bp, Artificial Sequence
  SNP primer
acgttggatgggcaagctacataatggaag

SEQ ID NO: 975 - DNA, 30 bp, Artificial Sequence
  SNP primer
acgttggatgtaacctccagtggatccatc

SEQ ID NO: 976 - DNA, 30 bp, Artificial Sequence
  SNP primer
acgttggatgtcccagagacttcttctacc

SEQ ID NO: 977 - DNA, 30 bp, Artificial Sequence
  SNP primer
acgttggatgcttcttttctactgtccagg

SEQ ID NO: 978 - DNA, 30 bp, Artificial Sequence
  SNP primer
acgttggatgtccggaccggaaccgcatct

SEQ ID NO: 979 - DNA, 31 bp, Artificial Sequence
  SNP primer
acgttggatgctccagcctggagtctttaaa

SEQ ID NO: 980 - DNA, 30 bp, Artificial Sequence
  SNP primer
acgttggatgactccctggccaaatgattg

SEQ ID NO: 981 - DNA, 30 bp, Artificial Sequence
  SNP primer
acgttggatgcctctcttctagctactttg

SEQ ID NO: 982 - DNA, 30 bp, Artificial Sequence
  SNP primer
acgttggatgagtgtctccaactatgtggc

SEQ ID NO: 983 - DNA, 30 bp, Artificial Sequence
  SNP primer
acgttggatgtaggagagagccaaatccag

SEQ ID NO: 984 - DNA, 30 bp, Artificial Sequence
  SNP primer
acgttggatgaaagctgagagaacccacag

SEQ ID NO: 985 - DNA, 30 bp, Artificial Sequence
  SNP primer
acgttggatgcatatggaagggtgtgcaag

SEQ ID NO: 986 - DNA, 30 bp, Artificial Sequence
  SNP primer
acgttggatgtaaagtctctgcgttctggc

SEQ ID NO: 987 - DNA, 30 bp, Artificial Sequence
  SNP primer
acgttggatgaattgttccctgagccagac

SEQ ID NO: 988 - DNA, 30 bp, Artificial Sequence
  SNP primer
acgttggatgggtaccccttgataaccatc

SEQ ID NO: 989 - DNA, 30 bp, Artificial Sequence
  SNP primer
acgttggatgaagcctccaagaaatttggg

SEQ ID NO: 990 - DNA, 30 bp, Artificial Sequence
  SNP primer
acgttggatgactgagaccacccatcatac

SEQ ID NO: 991 - DNA, 30 bp, Artificial Sequence
  SNP primer
acgttggatgagattaccacctatagactg

SEQ ID NO: 992 - DNA, 30 bp, Artificial Sequence
  SNP primer
acgttggatgtgttcttcctagaggcacag

SEQ ID NO: 993 - DNA, 30 bp, Artificial Sequence
  SNP primer
acgttggatgtccatttttgtcccctggac

SEQ ID NO: 994 - DNA, 30 bp, Artificial Sequence
  SNP primer
acgttggatgacaagaaggagatggtctgg

SEQ ID NO: 995 - DNA, 30 bp, Artificial Sequence
  SNP primer
acgttggatggcctaaaggtttctctgtac

SEQ ID NO: 996 - DNA, 30 bp, Artificial Sequence
  SNP primer
acgttggatgttcacggcttgactcaatgg

SEQ ID NO: 997 - DNA, 30 bp, Artificial Sequence
  SNP primer
acgttggatgaaggtggcactgattctagc

SEQ ID NO: 998 - DNA, 30 bp, Artificial Sequence
  SNP primer
acgttggatgatggctccagtttctgacac

SEQ ID NO: 999 - DNA, 30 bp, Artificial Sequence
  SNP primer
acgttggatgaagaaggcaagagcaggaag

SEQ ID NO: 1000 - DNA, 30 bp, Artificial Sequence
  SNP primer
acgttggatgttgtctcatttccagagccc

SEQ ID NO: 1001 - DNA, 30 bp, Artificial Sequence
  SNP primer
acgttggatgatccacagatgtgtgctcag

SEQ ID NO: 1002 - DNA, 30 bp, Artificial Sequence
  SNP primer
acgttggatgaagtgggtgttttgaccaag

SEQ ID NO: 1003 - DNA, 30 bp, Artificial Sequence
  SNP primer
acgttggatgtgcagaaccagtggaaaggg

SEQ ID NO: 1004 - DNA, 30 bp, Artificial Sequence
  SNP primer
acgttggatgtataccgtccaataggaggc

SEQ ID NO: 1005 - DNA, 30 bp, Artificial Sequence
  SNP primer
acgttggatgtgttttgccatgttggccag

SEQ ID NO: 1006 - DNA, 30 bp, Artificial Sequence
  SNP primer
acgttggatggcagcatggttttgtacaag

SEQ ID NO: 1007 - DNA, 30 bp, Artificial Sequence
  SNP primer
acgttggatgttaactgacctcgcccttgc

SEQ ID NO: 1008 - DNA, 30 bp, Artificial Sequence
  SNP primer
acgttggatggagttctttgcatcatctac

SEQ ID NO: 1009 - DNA, 30 bp, Artificial Sequence
  SNP primer
acgttggatggctctaagcatcattgtggg

SEQ ID NO: 1010 - DNA, 30 bp, Artificial Sequence
  SNP primer
acgttggatgtgcctcttctgtacttgggc

SEQ ID NO: 1011 - DNA, 30 bp, Artificial Sequence
  SNP primer
acgttggatgaaaagaaaccgcaccgctac

SEQ ID NO: 1012 - DNA, 30 bp, Artificial Sequence
  SNP primer
acgttggatgacgaacactaacgatggctg

SEQ ID NO: 1013 - DNA, 30 bp, Artificial Sequence
  SNP primer
acgttggatgagttctggaaataccttggg

SEQ ID NO: 1014 - DNA, 30 bp, Artificial Sequence
  SNP primer
acgttggatgctcctgactttaggtgatcc

SEQ ID NO: 1015 - DNA, 30 bp, Artificial Sequence
  SNP primer
acgttggatgaacacgttggatgttgtgcg

SEQ ID NO: 1016 - DNA, 30 bp, Artificial Sequence
  SNP primer
acgttggatgacgtgttagatgtgaggaag

SEQ ID NO: 1017 - DNA, 30 bp, Artificial Sequence
  SNP primer
acgttggatgttgggtgtttctcagagagg

SEQ ID NO: 1018 - DNA, 30 bp, Artificial Sequence
  SNP primer
acgttggatgatggtgcccgtacggtttgg

SEQ ID NO: 1019 - DNA, 30 bp, Artificial Sequence
  SNP primer
acgttggatggccataagatcagatgagcc

SEQ ID NO: 1020 - DNA, 30 bp, Artificial Sequence
  SNP primer
acgttggatgtttttgagacagagtctggc

SEQ ID NO: 1021 - DNA, 30 bp, Artificial Sequence
  SNP primer
acgttggatgtccccctttaccttccattg

SEQ ID NO: 1022 - DNA, 30 bp, Artificial Sequence
  SNP primer
acgttggatgaagtccagcttttcccctag

SEQ ID NO: 1023 - DNA, 30 bp, Artificial Sequence
  SNP primer
acgttggatggaaaaggtgactaaaaggtc

SEQ ID NO: 1024 - DNA, 30 bp, Artificial Sequence
  SNP primer
acgttggatgattggtcaagtctcccatgg

SEQ ID NO: 1025 - DNA, 30 bp, Artificial Sequence
  SNP primer
acgttggatgtgggagtctgatggactttc

SEQ ID NO: 1026 - DNA, 30 bp, Artificial Sequence
  SNP primer
acgttggatgaagcccaacaaggtattggg

SEQ ID NO: 1027 - DNA, 30 bp, Artificial Sequence
  SNP primer
acgttggatgagtttggggatgggagtcag

SEQ ID NO: 1028 - DNA, 30 bp, Artificial Sequence
  SNP primer
acgttggatgatcgagttcccacagcatag

SEQ ID NO: 1029 - DNA, 30 bp, Artificial Sequence
  SNP primer
acgttggatgatgcccaagccaagaaagag

SEQ ID NO: 1030 - DNA, 30 bp, Artificial Sequence
  SNP primer
acgttggatgtaacacggtgaaaccccgtc

SEQ ID NO: 1031 - DNA, 30 bp, Artificial Sequence
  SNP primer
acgttggatgactatcacaggaagcagtcg

SEQ ID NO: 1032 - DNA, 30 bp, Artificial Sequence
  SNP primer
acgttggatgcctccagtagcatgtaaggc

SEQ ID NO: 1033 - DNA, 30 bp, Artificial Sequence
  SNP primer
acgttggatgcgcataaagtagagggactc

SEQ ID NO: 1034 - DNA, 30 bp, Artificial Sequence
  SNP primer
acgttggatgaggctttccacacactcctg

SEQ ID NO: 1035 - DNA, 30 bp, Artificial Sequence
  SNP primer
acgttggatgtgtcttcaccacaggcagtg

SEQ ID NO: 1036 - DNA, 30 bp, Artificial Sequence
  SNP primer
acgttggatgaggagaattgcttgaacccg

SEQ ID NO: 1037 - DNA, 30 bp, Artificial Sequence
  SNP primer
acgttggatgatgacagtgcttccagagag

SEQ ID NO: 1038 - DNA, 30 bp, Artificial Sequence
  SNP primer
acgttggatgtctctgtgcctatgcatacc

SEQ ID NO: 1039 - DNA, 30 bp, Artificial Sequence
  SNP primer
acgttggatgcactagggaaatggttgtgc

SEQ ID NO: 1040 - DNA, 30 bp, Artificial Sequence
  SNP primer
acgttggatgattcatgtaaggcacggacc

SEQ ID NO: 1041 - DNA, 30 bp, Artificial Sequence
  SNP primer
acgttggatgtaactggataagctgccctc

SEQ ID NO: 1042 - DNA, 30 bp, Artificial Sequence
  SNP primer
acgttggatgttgctaaagtctcaggtggg

SEQ ID NO: 1043 - DNA, 30 bp, Artificial Sequence
  SNP primer
acgttggatgactacagggcttaggacttg

SEQ ID NO: 1044 - DNA, 30 bp, Artificial Sequence
  SNP primer
acgttggatggagaaacaaggtggtcacag

SEQ ID NO: 1045 - DNA, 30 bp, Artificial Sequence
  SNP primer
acgttggatgttttatgttgggctcccacc

SEQ ID NO: 1046 - DNA, 30 bp, Artificial Sequence
  SNP primer
acgttggatgtaacaaggccttgcccctag

SEQ ID NO: 1047 - DNA, 30 bp, Artificial Sequence
  SNP primer
acgttggatgaacatccctagactgggtcc

SEQ ID NO: 1048 - DNA, 30 bp, Artificial Sequence
  SNP primer
acgttggatgcagagagactgatggagaag

SEQ ID NO: 1049 - DNA, 30 bp, Artificial Sequence
  SNP primer
acgttggatgaagacctcagatagactggg

SEQ ID NO: 1050 - DNA, 31 bp, Artificial Sequence
  SNP primer
acgttggatgttcccccttgccccatatgag

SEQ ID NO: 1051 - DNA, 30 bp, Artificial Sequence
  SNP primer
acgttggatggagagcagaaaagagagagg

SEQ ID NO: 1052 - DNA, 30 bp, Artificial Sequence
  SNP primer
acgttggatgtcttcctttctccagactcc

SEQ ID NO: 1053 - DNA, 31 bp, Artificial Sequence
  SNP primer
acgttggatggctaagtaccgtgttttacag

SEQ ID NO: 1054 - DNA, 30 bp, Artificial Sequence
  SNP primer
acgttggatgttatcccttaccctaggtgg

SEQ ID NO: 1055 - DNA, 30 bp, Artificial Sequence
  SNP primer
acgttggatgtaacagcccttatagggtgg

SEQ ID NO: 1056 - DNA, 30 bp, Artificial Sequence
  SNP primer
acgttggatgaggaaggaagcctgttgcag

SEQ ID NO: 1057 - DNA, 30 bp, Artificial Sequence
  SNP primer
acgttggatgtaccagctgaagctctttgc

SEQ ID NO: 1058 - DNA, 30 bp, Artificial Sequence
  SNP primer
acgttggatgtgagaagagtgggcatgatg

SEQ ID NO: 1059 - DNA, 30 bp, Artificial Sequence
  SNP primer
acgttggatggggaggaagccaaaaatagc

SEQ ID NO: 1060 - DNA, 30 bp, Artificial Sequence
  SNP primer
acgttggatgtcctcttacaccccatatcc

SEQ ID NO: 1061 - DNA, 30 bp, Artificial Sequence
  SNP primer
acgttggatgtggcccaggttatacgatac

SEQ ID NO: 1062 - DNA, 30 bp, Artificial Sequence
  SNP primer
acgttggatgcctgcgtggtttaaaagctc

SEQ ID NO: 1063 - DNA, 30 bp, Artificial Sequence
  SNP primer
acgttggatggctgaacaaatcagctctgg

SEQ ID NO: 1064 - DNA, 30 bp, Artificial Sequence
  SNP primer
acgttggatgcacacttccctctcactgtg

SEQ ID NO: 1065 - DNA, 30 bp, Artificial Sequence
  SNP primer
acgttggatgttctgattcacaggtctggc

SEQ ID NO: 1066 - DNA, 30 bp, Artificial Sequence
  SNP primer
acgttggatgagacagccagagcaactcag

SEQ ID NO: 1067 - DNA, 30 bp, Artificial Sequence
  SNP primer
acgttggatgtttcagaagctgcacctgac

SEQ ID NO: 1068 - DNA, 30 bp, Artificial Sequence
  SNP primer
acgttggatggactggcatgtccagctgtc

SEQ ID NO: 1069 - DNA, 30 bp, Artificial Sequence
  SNP primer
acgttggatgtgcaaacctctcagagtctc

SEQ ID NO: 1070 - DNA, 30 bp, Artificial Sequence
  SNP primer
acgttggatgaaccaatccctgggatggag

SEQ ID NO: 1071 - DNA, 30 bp, Artificial Sequence; identical to SEQ ID NO: 1070
  SNP primer

SEQ ID NO: 1072 - DNA, 30 bp, Artificial Sequence
  SNP primer
acgttggatgtttgcatctgtggagagctc

SEQ ID NO: 1073 - DNA, 30 bp, Artificial Sequence
  SNP primer
acgttggatgctttgaagtaggaggatctg

SEQ ID NO: 1074 - DNA, 30 bp, Artificial Sequence
  SNP primer
acgttggatgtttcccactaggactcttcc

SEQ ID NO: 1075 - DNA, 30 bp, Artificial Sequence
  SNP primer
acgttggatggactcggcttctgaatatgc

SEQ ID NO: 1076 - DNA, 30 bp, Artificial Sequence
  SNP primer
acgttggatgaactatgacacacccgaagc

SEQ ID NO: 1077 - DNA, 30 bp, Artificial Sequence
  SNP primer
acgttggatgttacaacagccctgaggttc

SEQ ID NO: 1078 - DNA, 30 bp, Artificial Sequence
  SNP primer
acgttggatgatatccgtggcaattcccac

SEQ ID NO: 1079 - DNA, 18 bp, Artificial Sequence
  SNP primer
actgggtgcatcagggag

SEQ ID NO: 1080 - DNA, 16 bp, Artificial Sequence
  SNP primer
ctccagtccccactgc

SEQ ID NO: 1081 - DNA, 19 bp, Artificial Sequence
  SNP primer
  misc_feature 14OTHER INFORMATION: n = A,T,C or G
ggtgtcagagcaanattcc

SEQ ID NO: 1082 - DNA, 22 bp, Artificial Sequence
ggagacacattcaaaccatagc

SEQ ID NO: 1083 - DNA, 20 bp, Artificial Sequence
  SNP primer
aatggctgttaggaagaagc

SEQ ID NO: 1084 - DNA, 28 bp, Artificial Sequence
  SNP primer
aatagccatgagaagctatgtgggggag

SEQ ID NO: 1085 - DNA, 20 bp, Artificial Sequence
  SNP primer
gtgaaatcagcctgcttctg

SEQ ID NO: 1086 - DNA, 22 bp, Artificial Sequence
  SNP primer
agatggcatttggagagtgcag

SEQ ID NO: 1087 - DNA, 20 bp, Artificial Sequence
  SNP primer
acgttcgtacccattaacct

SEQ ID NO: 1088 - DNA, 21 bp, Artificial Sequence
  SNP primer
gaaatgtgagaataaaggaga

SEQ ID NO: 1089 - DNA, 22 bp, Artificial Sequence
  SNP primer
gcaaatcaagaatgtaattccc

SEQ ID NO: 1090 - DNA, 19 bp, Artificial Sequence
  SNP primer
ctttacaacccttggcagc

SEQ ID NO: 1091 - DNA, 16 bp, Artificial Sequence
  SNP primer
cccaaggccacatagc

SEQ ID NO: 1092 - DNA, 23 bp, Artificial Sequence
  SNP primer
aacaaagtatcacaaactgggag

SEQ ID NO: 1093 - DNA, 24 bp, Artificial Sequence
  SNP primer
caattggtgacatacatcaacttg

SEQ ID NO: 1094 - DNA, 19 bp, Artificial Sequence
  SNP primer
tcttccttagctgctgctg

SEQ ID NO: 1095 - DNA, 29 bp, Artificial Sequence
  SNP primer
ctacctccttgccaaacttgctgtttgtg

SEQ ID NO: 1096 - DNA, 20 bp, Artificial Sequence
  SNP primer
gaacacaaccatctctgctc

SEQ ID NO: 1097 - DNA, 22 bp, Artificial Sequence
  SNP primer
tccttacagcagagatatgtgg

SEQ ID NO: 1098 - DNA, 20 bp, Artificial Sequence
  SNP primer
atcgaggtaaacagcagaaa

SEQ ID NO: 1099 - DNA, 20 bp, Artificial Sequence
  SNP primer
gataaaggggaactactaca

SEQ ID NO: 1100 - DNA, 20 bp, Artificial Sequence
  SNP primer
cttccaatccataagcatgg